SEQUENCE LISTING (1111 sequences)

SEQ ID NO: 1 - protein, 7 aa, synthetic construct
  REGION 1..7: note = Synthetic
NNSTRGG

SEQ ID NO: 2 - protein, 7 aa, synthetic construct
  REGION 1..7: note = Synthetic
GNSARNI

SEQ ID NO: 3 - protein, 7 aa, synthetic construct
  REGION 1..7: note = Synthetic
GNSVRDF

SEQ ID NO: 4 - protein, 15 aa, synthetic construct
  REGION 1..15: note = Synthetic
GGGGGGGGGGGGGGG

SEQ ID NO: 5 - protein, 15 aa, synthetic construct
  REGION 1..15: note = Synthetic
GGGGSGGGGSGGGGS

SEQ ID NO: 6 - protein, 7 aa, synthetic construct
  REGION 1..7: note = Synthetic
ANAAKNY

SEQ ID NO: 7 - protein, 7 aa, synthetic construct
  REGION 1..7: note = Synthetic
FNGIKEM

SEQ ID NO: 8 - protein, 7 aa, synthetic construct
  REGION 1..7: note = Synthetic
NNHTREV

SEQ ID NO: 9 - protein, 7 aa, synthetic construct
  REGION 1..7: note = Synthetic
TNKIRDP

SEQ ID NO: 10 - protein, 7 aa, synthetic construct
  REGION 1..7: note = Synthetic
ANALKNQ

SEQ ID NO: 11 - protein, 7 aa, synthetic construct
  REGION 1..7: note = Synthetic
FNKDKED

SEQ ID NO: 12 - protein, 7 aa, synthetic construct
  REGION 1..7: note = Synthetic
NNMIRAV

SEQ ID NO: 13 - protein, 7 aa, synthetic construct
  REGION 1..7: note = Synthetic
TNNTRQN

SEQ ID NO: 14 - protein, 7 aa, synthetic construct
  REGION 1..7: note = Synthetic
ANATRLQ

SEQ ID NO: 15 - protein, 7 aa, synthetic construct
  REGION 1..7: note = Synthetic
FNSTRPD

SEQ ID NO: 16 - protein, 7 aa, synthetic construct
  REGION 1..7: note = Synthetic
NNNTKNI

SEQ ID NO: 17 - protein, 7 aa, synthetic construct
  REGION 1..7: note = Synthetic
TNNTRVG

SEQ ID NO: 18 - protein, 7 aa, synthetic construct
  REGION 1..7: note = Synthetic
ANATRYQ

SEQ ID NO: 19 - protein, 7 aa, synthetic construct
  REGION 1..7: note = Synthetic
GNATKAG

SEQ ID NO: 20 - protein, 7 aa, synthetic construct
  REGION 1..7: note = Synthetic
NNNTRNH

SEQ ID NO: 21 - protein, 7 aa, synthetic construct
  REGION 1..7: note = Synthetic
TNNVKSG

SEQ ID NO: 22 - protein, 7 aa, synthetic construct
  REGION 1..7: note = Synthetic
ANEYKAH

SEQ ID NO: 23 - protein, 7 aa, synthetic construct
  REGION 1..7: note = Synthetic
GNDVKDI

SEQ ID NO: 24 - protein, 7 aa, synthetic construct
  REGION 1..7: note = Synthetic
NNQTRMM

SEQ ID NO: 25 - protein, 7 aa, synthetic construct
  REGION 1..7: note = Synthetic
TNPDRSA

SEQ ID NO: 26 - protein, 7 aa, synthetic construct
  REGION 1..7: note = Synthetic
ANGTKNG

SEQ ID NO: 27 - protein, 7 aa, synthetic construct
  REGION 1..7: note = Synthetic
GNETRYT

SEQ ID NO: 28 - protein, 7 aa, synthetic construct
  REGION 1..7: note = Synthetic
NNSDKVK

SEQ ID NO: 29 - protein, 7 aa, synthetic construct
  REGION 1..7: note = Synthetic
TNQTKVH

SEQ ID NO: 30 - protein, 7 aa, synthetic construct
  REGION 1..7: note = Synthetic
ANIVKLQ

SEQ ID NO: 31 - protein, 7 aa, synthetic construct
  REGION 1..7: note = Synthetic
GNFVKSG

SEQ ID NO: 32 - protein, 7 aa, synthetic construct
  REGION 1..7: note = Synthetic
NNSIKSQ

SEQ ID NO: 33 - protein, 7 aa, synthetic construct
  REGION 1..7: note = Synthetic
TNSAKPY

SEQ ID NO: 34 - protein, 7 aa, synthetic construct
  REGION 1..7: note = Synthetic
ANLAKVM

SEQ ID NO: 35 - protein, 7 aa, synthetic construct
  REGION 1..7: note = Synthetic
GNHSKIQ

SEQ ID NO: 36 - protein, 7 aa, synthetic construct
  REGION 1..7: note = Synthetic
NNSTRTA

SEQ ID NO: 37 - protein, 7 aa, synthetic construct
  REGION 1..7: note = Synthetic
TNSTKGA

SEQ ID NO: 38 - protein, 7 aa, synthetic construct
  REGION 1..7: note = Synthetic
ANLVKTT

SEQ ID NO: 39 - protein, 7 aa, synthetic construct
  REGION 1..7: note = Synthetic
GNIIRVH

SEQ ID NO: 40 - protein, 7 aa, synthetic construct
  REGION 1..7: note = Synthetic
NNTIKNG

SEQ ID NO: 41 - protein, 7 aa, synthetic construct
  REGION 1..7: note = Synthetic
TNSTRIG

SEQ ID NO: 42 - protein, 7 aa, synthetic construct
  REGION 1..7: note = Synthetic
ANPNKSV

SEQ ID NO: 43 - protein, 7 aa, synthetic construct
  REGION 1..7: note = Synthetic
GNLPKIA

SEQ ID NO: 44 - protein, 7 aa, synthetic construct
  REGION 1..7: note = Synthetic
NNVVKNI

SEQ ID NO: 45 - protein, 7 aa, synthetic construct
  REGION 1..7: note = Synthetic
TNSVKAH

SEQ ID NO: 46 - protein, 7 aa, synthetic construct
  REGION 1..7: note = Synthetic
ANSERFL

SEQ ID NO: 47 - protein, 7 aa, synthetic construct
  REGION 1..7: note = Synthetic
GNNIRTQ

SEQ ID NO: 48 - protein, 7 aa, synthetic construct
  REGION 1..7: note = Synthetic
NNYTKHQ

SEQ ID NO: 49 - protein, 7 aa, synthetic construct
  REGION 1..7: note = Synthetic
TNTLKNV

SEQ ID NO: 50 - protein, 7 aa, synthetic construct
  REGION 1..7: note = Synthetic
ANSIRDV

SEQ ID NO: 51 - protein, 7 aa, synthetic construct
  REGION 1..7: note = Synthetic
GNNSKPP

SEQ ID NO: 52 - protein, 7 aa, synthetic construct
  REGION 1..7: note = Synthetic
NNYTRPI

SEQ ID NO: 53 - protein, 7 aa, synthetic construct
  REGION 1..7: note = Synthetic
TNTTRSP

SEQ ID NO: 54 - protein, 7 aa, synthetic construct
  REGION 1..7: note = Synthetic
ANSTRTW

SEQ ID NO: 55 - protein, 7 aa, synthetic construct
  REGION 1..7: note = Synthetic
GNNTKMG

SEQ ID NO: 56 - protein, 7 aa, synthetic construct
  REGION 1..7: note = Synthetic
PNALRMV

SEQ ID NO: 57 - protein, 7 aa, synthetic construct
  REGION 1..7: note = Synthetic
VNAIRDY

SEQ ID NO: 58 - protein, 7 aa, synthetic construct
  REGION 1..7: note = Synthetic
ANTTKIE

SEQ ID NO: 59 - protein, 7 aa, synthetic construct
  REGION 1..7: note = Synthetic
GNPEKND

SEQ ID NO: 60 - protein, 7 aa, synthetic construct
  REGION 1..7: note = Synthetic
PNEQKVI

SEQ ID NO: 61 - protein, 7 aa, synthetic construct
  REGION 1..7: note = Synthetic
VNATRVY

SEQ ID NO: 62 - protein, 7 aa, synthetic construct
  REGION 1..7: note = Synthetic
ANVVKQF

SEQ ID NO: 63 - protein, 7 aa, synthetic construct
  REGION 1..7: note = Synthetic
GNQTKNG

SEQ ID NO: 64 - protein, 7 aa, synthetic construct
  REGION 1..7: note = Synthetic
PNIIKQN

SEQ ID NO: 65 - protein, 7 aa, synthetic construct
  REGION 1..7: note = Synthetic
VNGGKFM

SEQ ID NO: 66 - protein, 7 aa, synthetic construct
  REGION 1..7: note = Synthetic
CNEGRSC

SEQ ID NO: 67 - protein, 7 aa, synthetic construct
  REGION 1..7: note = Synthetic
GNQTRGG

SEQ ID NO: 68 - protein, 7 aa, synthetic construct
  REGION 1..7: note = Synthetic
PNLSKER

SEQ ID NO: 69 - protein, 7 aa, synthetic construct
  REGION 1..7: note = Synthetic
VNITKNF

SEQ ID NO: 70 - protein, 7 aa, synthetic construct
  REGION 1..7: note = Synthetic
DNDHRDI

SEQ ID NO: 71 - protein, 7 aa, synthetic construct
  REGION 1..7: note = Synthetic
GNQVKGG

SEQ ID NO: 72 - protein, 7 aa, synthetic construct
  REGION 1..7: note = Synthetic
PNMQKQI

SEQ ID NO: 73 - protein, 7 aa, synthetic construct
  REGION 1..7: note = Synthetic
VNITKSW

SEQ ID NO: 74 - protein, 7 aa, synthetic construct
  REGION 1..7: note = Synthetic
DNDSKSS

SEQ ID NO: 75 - protein, 7 aa, synthetic construct
  REGION 1..7: note = Synthetic
GNRSKDY

SEQ ID NO: 76 - protein, 7 aa, synthetic construct
  REGION 1..7: note = Synthetic
PNSDKLK

SEQ ID NO: 77 - protein, 7 aa, synthetic construct
  REGION 1..7: note = Synthetic
VNMTKPI

SEQ ID NO: 78 - protein, 7 aa, synthetic construct
  REGION 1..7: note = Synthetic
DNDVKGG

SEQ ID NO: 79 - protein, 7 aa, synthetic construct
  REGION 1..7: note = Synthetic
GNSVKNG

SEQ ID NO: 80 - protein, 7 aa, synthetic construct
  REGION 1..7: note = Synthetic
PNVAKQQ

SEQ ID NO: 81 - protein, 7 aa, synthetic construct
  REGION 1..7: note = Synthetic
VNMVRTF

SEQ ID NO: 82 - protein, 7 aa, synthetic construct
  REGION 1..7: note = Synthetic
DNGTKTF

SEQ ID NO: 83 - protein, 7 aa, synthetic construct
  REGION 1..7: note = Synthetic
GNTIRQA

SEQ ID NO: 84 - protein, 7 aa, synthetic construct
  REGION 1..7: note = Synthetic
QNALRNI

SEQ ID NO: 85 - protein, 7 aa, synthetic construct
  REGION 1..7: note = Synthetic
VNQVKNH

SEQ ID NO: 86 - protein, 7 aa, synthetic construct
  REGION 1..7: note = Synthetic
DNGVRGY

SEQ ID NO: 87 - protein, 7 aa, synthetic construct
  REGION 1..7: note = Synthetic
GNTVKGS

SEQ ID NO: 88 - protein, 7 aa, synthetic construct
  REGION 1..7: note = Synthetic
QNEVRNG

SEQ ID NO: 89 - protein, 7 aa, synthetic construct
  REGION 1..7: note = Synthetic
VNVERKL

SEQ ID NO: 90 - protein, 7 aa, synthetic construct
  REGION 1..7: note = Synthetic
DNHIKPS

SEQ ID NO: 91 - protein, 7 aa, synthetic construct
  REGION 1..7: note = Synthetic
GNVVKST

SEQ ID NO: 92 - protein, 7 aa, synthetic construct
  REGION 1..7: note = Synthetic
QNFVKIS

SEQ ID NO: 93 - protein, 7 aa, synthetic construct
  REGION 1..7: note = Synthetic
VNVGKGP

SEQ ID NO: 94 - protein, 7 aa, synthetic construct
  REGION 1..7: note = Synthetic
DNIFKEF

SEQ ID NO: 95 - protein, 7 aa, synthetic construct
  REGION 1..7: note = Synthetic
HNANKML

SEQ ID NO: 96 - protein, 7 aa, synthetic construct
  REGION 1..7: note = Synthetic
QNLGKFI

SEQ ID NO: 97 - protein, 7 aa, synthetic construct
  REGION 1..7: note = Synthetic
WNGNRAA

SEQ ID NO: 98 - protein, 7 aa, synthetic construct
  REGION 1..7: note = Synthetic
DNKIKST

SEQ ID NO: 99 - protein, 7 aa, synthetic construct
  REGION 1..7: note = Synthetic
HNEPKNH

SEQ ID NO: 100 - protein, 7 aa, synthetic construct
  REGION 1..7: note = Synthetic
QNMDKPA

SEQ ID NO: 101 - protein, 7 aa, synthetic construct
  REGION 1..7: note = Synthetic
YNATRGG

SEQ ID NO: 102 - protein, 7 aa, synthetic construct
  REGION 1..7: note = Synthetic
DNKLRPI

SEQ ID NO: 103 - protein, 7 aa, synthetic construct
  REGION 1..7: note = Synthetic
HNLDKTH

SEQ ID NO: 104 - protein, 7 aa, synthetic construct
  REGION 1..7: note = Synthetic
QNMVRGN

SEQ ID NO: 105 - protein, 7 aa, synthetic construct
  REGION 1..7: note = Synthetic
YNSTRNG

SEQ ID NO: 106 - protein, 7 aa, synthetic construct
  REGION 1..7: note = Synthetic
DNMIRKV

SEQ ID NO: 107 - protein, 7 aa, synthetic construct
  REGION 1..7: note = Synthetic
HNLNKFT

SEQ ID NO: 108 - protein, 7 aa, synthetic construct
  REGION 1..7: note = Synthetic
QNQTRIQ

SEQ ID NO: 109 - protein, 7 aa, synthetic construct
  REGION 1..7: note = Synthetic
YNTQKEI

SEQ ID NO: 110 - protein, 7 aa, synthetic construct
  REGION 1..7: note = Synthetic
DNPSRSV

SEQ ID NO: 111 - protein, 7 aa, synthetic construct
  REGION 1..7: note = Synthetic
HNPIRFP

SEQ ID NO: 112 - protein, 7 aa, synthetic construct
  REGION 1..7: note = Synthetic
QNTIRMY

SEQ ID NO: 113 - protein, 7 aa, synthetic construct
  REGION 1..7: note = Synthetic
YNTTKDK

SEQ ID NO: 114 - protein, 7 aa, synthetic construct
  REGION 1..7: note = Synthetic
DNQMKFV

SEQ ID NO: 115 - protein, 7 aa, synthetic construct
  REGION 1..7: note = Synthetic
INKEKFT

SEQ ID NO: 116 - protein, 7 aa, synthetic construct
  REGION 1..7: note = Synthetic
QNTTKAG

SEQ ID NO: 117 - protein, 7 aa, synthetic construct
  REGION 1..7: note = Synthetic
DNTERIP

SEQ ID NO: 118 - protein, 7 aa, synthetic construct
  REGION 1..7: note = Synthetic
INSDKSL

SEQ ID NO: 119 - protein, 7 aa, synthetic construct
  REGION 1..7: note = Synthetic
QNYTKNN

SEQ ID NO: 120 - protein, 7 aa, synthetic construct
  REGION 1..7: note = Synthetic
DNTERPA

SEQ ID NO: 121 - protein, 7 aa, synthetic construct
  REGION 1..7: note = Synthetic
KNDLRNE

SEQ ID NO: 122 - protein, 7 aa, synthetic construct
  REGION 1..7: note = Synthetic
RNDTKGG

SEQ ID NO: 123 - protein, 7 aa, synthetic construct
  REGION 1..7: note = Synthetic
DNTNKEE

SEQ ID NO: 124 - protein, 7 aa, synthetic construct
  REGION 1..7: note = Synthetic
KNEPKNL

SEQ ID NO: 125 - protein, 7 aa, synthetic construct
  REGION 1..7: note = Synthetic
SNAIKLS

SEQ ID NO: 126 - protein, 7 aa, synthetic construct
  REGION 1..7: note = Synthetic
ENADKGR

SEQ ID NO: 127 - protein, 7 aa, synthetic construct
  REGION 1..7: note = Synthetic
KNEVKNA

SEQ ID NO: 128 - protein, 7 aa, synthetic construct
  REGION 1..7: note = Synthetic
SNAPRIV

SEQ ID NO: 129 - protein, 7 aa, synthetic construct
  REGION 1..7: note = Synthetic
ENDPKAV

SEQ ID NO: 130 - protein, 7 aa, synthetic construct
  REGION 1..7: note = Synthetic
LNAMRPL

SEQ ID NO: 131 - protein, 7 aa, synthetic construct
  REGION 1..7: note = Synthetic
SNAPRQV

SEQ ID NO: 132 - protein, 7 aa, synthetic construct
  REGION 1..7: note = Synthetic
ENGLKYP

SEQ ID NO: 133 - protein, 7 aa, synthetic construct
  REGION 1..7: note = Synthetic
LNGERQI

SEQ ID NO: 134 - protein, 7 aa, synthetic construct
  REGION 1..7: note = Synthetic
SNAVKSA

SEQ ID NO: 135 - protein, 7 aa, synthetic construct
  REGION 1..7: note = Synthetic
ENHVKGQ

SEQ ID NO: 136 - protein, 7 aa, synthetic construct
  REGION 1..7: note = Synthetic
LNKDKLI

SEQ ID NO: 137 - protein, 7 aa, synthetic construct
  REGION 1..7: note = Synthetic
SNDIRNN

SEQ ID NO: 138 - protein, 7 aa, synthetic construct
  REGION 1..7: note = Synthetic
ENKERDV

SEQ ID NO: 139 - protein, 7 aa, synthetic construct
  REGION 1..7: note = Synthetic
LNLNRLE

SEQ ID NO: 140 - protein, 7 aa, synthetic construct
  REGION 1..7: note = Synthetic
SNDKRPL

SEQ ID NO: 141 - protein, 7 aa, synthetic construct
  REGION 1..7: note = Synthetic
ENKIKLV

SEQ ID NO: 142 - protein, 7 aa, synthetic construct
  REGION 1..7: note = Synthetic
MNGSKTA

SEQ ID NO: 143 - protein, 7 aa, synthetic construct
  REGION 1..7: note = Synthetic
SNHIKSA

SEQ ID NO: 144 - protein, 7 aa, synthetic construct
  REGION 1..7: note = Synthetic
ENLERLK

SEQ ID NO: 145 - protein, 7 aa, synthetic construct
  REGION 1..7: note = Synthetic
MNQQKLV

SEQ ID NO: 146 - protein, 7 aa, synthetic construct
  REGION 1..7: note = Synthetic
SNHTKNF

SEQ ID NO: 147 - protein, 7 aa, synthetic construct
  REGION 1..7: note = Synthetic
ENNVKAV

SEQ ID NO: 148 - protein, 7 aa, synthetic construct
  REGION 1..7: note = Synthetic
MNQTRGG

SEQ ID NO: 149 - protein, 7 aa, synthetic construct
  REGION 1..7: note = Synthetic
SNLQKFI

SEQ ID NO: 150 - protein, 7 aa, synthetic construct
  REGION 1..7: note = Synthetic
ENPIKST

SEQ ID NO: 151 - protein, 7 aa, synthetic construct
  REGION 1..7: note = Synthetic
MNSTKNG

SEQ ID NO: 152 - protein, 7 aa, synthetic construct
  REGION 1..7: note = Synthetic
SNLQKVE

SEQ ID NO: 153 - protein, 7 aa, synthetic construct
  REGION 1..7: note = Synthetic
ENQIRSL

SEQ ID NO: 154 - protein, 7 aa, synthetic construct
  REGION 1..7: note = Synthetic
MNSVRFM

SEQ ID NO: 155 - protein, 7 aa, synthetic construct
  REGION 1..7: note = Synthetic
SNQVRAL

SEQ ID NO: 156 - protein, 7 aa, synthetic construct
  REGION 1..7: note = Synthetic
ENQVKQY

SEQ ID NO: 157 - protein, 7 aa, synthetic construct
  REGION 1..7: note = Synthetic
MNVTKQT

SEQ ID NO: 158 - protein, 7 aa, synthetic construct
  REGION 1..7: note = Synthetic
SNSTKSA

SEQ ID NO: 159 - protein, 7 aa, synthetic construct
  REGION 1..7: note = Synthetic
ENQVRME

SEQ ID NO: 160 - protein, 7 aa, synthetic construct
  REGION 1..7: note = Synthetic
NNATRAA

SEQ ID NO: 161 - protein, 7 aa, synthetic construct
  REGION 1..7: note = Synthetic
SNSTRAP

SEQ ID NO: 162 - protein, 7 aa, synthetic construct
  REGION 1..7: note = Synthetic
ENQYKAI

SEQ ID NO: 163 - protein, 7 aa, synthetic construct
  REGION 1..7: note = Synthetic
NNATRPA

SEQ ID NO: 164 - protein, 7 aa, synthetic construct
  REGION 1..7: note = Synthetic
SNTTRPI

SEQ ID NO: 165 - protein, 7 aa, synthetic construct
  REGION 1..7: note = Synthetic
ENTSRTQ

SEQ ID NO: 166 - protein, 7 aa, synthetic construct
  REGION 1..7: note = Synthetic
NNGTRLS

SEQ ID NO: 167 - protein, 7 aa, synthetic construct
  REGION 1..7: note = Synthetic
SNVNRLV

SEQ ID NO: 168 - protein, 7 aa, synthetic construct
  REGION 1..7: note = Synthetic
ENVERLK

SEQ ID NO: 169 - protein, 7 aa, synthetic construct
  REGION 1..7: note = Synthetic
NNHDKSK

SEQ ID NO: 170 - protein, 7 aa, synthetic construct
  REGION 1..7: note = Synthetic
SNVTKWV

SEQ ID NO: 171 - protein, 7 aa, synthetic construct
  REGION 1..7: note = Synthetic
ENVIRSV

SEQ ID NO: 172 - protein, 7 aa, synthetic construct
  REGION 1..7: note = Synthetic
NNHIRET

SEQ ID NO: 173 - protein, 7 aa, synthetic construct
  REGION 1..7: note = Synthetic
TNATKGG

SEQ ID NO: 174 - protein, 7 aa, synthetic construct
  REGION 1..7: note = Synthetic
FNAERMT

SEQ ID NO: 175 - protein, 7 aa, synthetic construct
  REGION 1..7: note = Synthetic
NNHIRYH

SEQ ID NO: 176 - protein, 7 aa, synthetic construct
  REGION 1..7: note = Synthetic
TNDDKYQ

SEQ ID NO: 177 - protein, 7 aa, synthetic construct
  REGION 1..7: note = Synthetic
FNDAKGT

SEQ ID NO: 178 - protein, 7 aa, synthetic construct
  REGION 1..7: note = Synthetic
NNHTKDI

SEQ ID NO: 179 - protein, 7 aa, synthetic construct
  REGION 1..7: note = Synthetic
TNDVRRL

SEQ ID NO: 180 - protein, 7 aa, synthetic construct
  REGION 1..7: note = Synthetic
FNDAKQW

SEQ ID NO: 181 - protein, 7 aa, synthetic construct
  REGION 1..7: note = Synthetic
NNHTKGV

SEQ ID NO: 182 - protein, 7 aa, synthetic construct
  REGION 1..7: note = Synthetic
TNEDKGL

SEQ ID NO: 183 - protein, 7 aa, synthetic construct
  REGION 1..7: note = Synthetic
ANAVRAI

SEQ ID NO: 184 - protein, 7 aa, synthetic construct
  REGION 1..7: note = Synthetic
GNSIRDT

SEQ ID NO: 185 - protein, 7 aa, synthetic construct
  REGION 1..7: note = Synthetic
LNSSRIP

SEQ ID NO: 186 - protein, 7 aa, synthetic construct
  REGION 1..7: note = Synthetic
SNAIKST

SEQ ID NO: 187 - protein, 7 aa, synthetic construct
  REGION 1..7: note = Synthetic
ANDIRPW

SEQ ID NO: 188 - protein, 7 aa, synthetic construct
  REGION 1..7: note = Synthetic
GNSIRTI

SEQ ID NO: 189 - protein, 7 aa, synthetic construct
  REGION 1..7: note = Synthetic
LNSVRHV

SEQ ID NO: 190 - protein, 7 aa, synthetic construct
  REGION 1..7: note = Synthetic
SNATRQV

SEQ ID NO: 191 - protein, 7 aa, synthetic construct
  REGION 1..7: note = Synthetic
ANHIRSL

SEQ ID NO: 192 - protein, 7 aa, synthetic construct
  REGION 1..7: note = Synthetic
GNSLRAY

SEQ ID NO: 193 - protein, 7 aa, synthetic construct
  REGION 1..7: note = Synthetic
LNTIRST

SEQ ID NO: 194 - protein, 7 aa, synthetic construct
  REGION 1..7: note = Synthetic
SNATRSI

SEQ ID NO: 195 - protein, 7 aa, synthetic construct
  REGION 1..7: note = Synthetic
ANTGRHN

SEQ ID NO: 196 - protein, 7 aa, synthetic construct
  REGION 1..7: note = Synthetic
GNSMRHM

SEQ ID NO: 197 - protein, 7 aa, synthetic construct
  REGION 1..7: note = Synthetic
LNTIRTN

SEQ ID NO: 198 - protein, 7 aa, synthetic construct
  REGION 1..7: note = Synthetic
SNEIRLV

SEQ ID NO: 199 - protein, 7 aa, synthetic construct
  REGION 1..7: note = Synthetic
ANYARND

SEQ ID NO: 200 - protein, 7 aa, synthetic construct
  REGION 1..7: note = Synthetic
GNSSRNL

SEQ ID NO: 201 - protein, 7 aa, synthetic construct
  REGION 1..7: note = Synthetic
LNTTRPI

SEQ ID NO: 202 - protein, 7 aa, synthetic construct
  REGION 1..7: note = Synthetic
SNFIRSA

SEQ ID NO: 203 - protein, 7 aa, synthetic construct
  REGION 1..7: note = Synthetic
DNAVRPW

SEQ ID NO: 204 - protein, 7 aa, synthetic construct
  REGION 1..7: note = Synthetic
GNSTRDH

SEQ ID NO: 205 - protein, 7 aa, synthetic construct
  REGION 1..7: note = Synthetic
LNTTRQV

SEQ ID NO: 206 - protein, 7 aa, synthetic construct
  REGION 1..7: note = Synthetic
SNHIRLA

SEQ ID NO: 207 - protein, 7 aa, synthetic construct
  REGION 1..7: note = Synthetic
DNEIRRW

SEQ ID NO: 208 - protein, 7 aa, synthetic construct
  REGION 1..7: note = Synthetic
GNSVKPS

SEQ ID NO: 209 - protein, 7 aa, synthetic construct
  REGION 1..7: note = Synthetic
LNTVRDI

SEQ ID NO: 210 - protein, 7 aa, synthetic construct
  REGION 1..7: note = Synthetic
SNHIRTL

SEQ ID NO: 211 - protein, 7 aa, synthetic construct
  REGION 1..7: note = Synthetic
DNHIRSQ

SEQ ID NO: 212 - protein, 7 aa, synthetic construct
  REGION 1..7: note = Synthetic
GNSVKQF

SEQ ID NO: 213 - protein, 7 aa, synthetic construct
  REGION 1..7: note = Synthetic
LNTVREP

SEQ ID NO: 214 - protein, 7 aa, synthetic construct
  REGION 1..7: note = Synthetic
SNHVRAI

SEQ ID NO: 215 - protein, 7 aa, synthetic construct
  REGION 1..7: note = Synthetic
DNHVKSL

SEQ ID NO: 216 - protein, 7 aa, synthetic construct
  REGION 1..7: note = Synthetic
GNSVRDF

SEQ ID NO: 217 - protein, 7 aa, synthetic construct
  REGION 1..7: note = Synthetic
LNTVRHI

SEQ ID NO: 218 - protein, 7 aa, synthetic construct
  REGION 1..7: note = Synthetic
SNHVRFM

SEQ ID NO: 219 - protein, 7 aa, synthetic construct
  REGION 1..7: note = Synthetic
DNKVKPT

SEQ ID NO: 220 - protein, 7 aa, synthetic construct
  REGION 1..7: note = Synthetic
GNSVRGW

SEQ ID NO: 221 - protein, 7 aa, synthetic construct
  REGION 1..7: note = Synthetic
LNVIRDP

SEQ ID NO: 222 - protein, 7 aa, synthetic construct
  REGION 1..7: note = Synthetic
SNHVRTS

SEQ ID NO: 223 - protein, 7 aa, synthetic construct
  REGION 1..7: note = Synthetic
DNNTRSV

SEQ ID NO: 224 - protein, 7 aa, synthetic construct
  REGION 1..7: note = Synthetic
GNSVRPV

SEQ ID NO: 225 - protein, 7 aa, synthetic construct
  REGION 1..7: note = Synthetic
LNYTRSS

SEQ ID NO: 226 - protein, 7 aa, synthetic construct
  REGION 1..7: note = Synthetic
SNNGRYM

SEQ ID NO: 227 - protein, 7 aa, synthetic construct
  REGION 1..7: note = Synthetic
DNRTKAL

SEQ ID NO: 228 - protein, 7 aa, synthetic construct
  REGION 1..7: note = Synthetic
GNTIRDI

SEQ ID NO: 229 - protein, 7 aa, synthetic construct
  REGION 1..7: note = Synthetic
MNDVRSV

SEQ ID NO: 230 - protein, 7 aa, synthetic construct
  REGION 1..7: note = Synthetic
SNNVRHY

SEQ ID NO: 231 - protein, 7 aa, synthetic construct
  REGION 1..7: note = Synthetic
DNRVRSP

SEQ ID NO: 232 - protein, 7 aa, synthetic construct
  REGION 1..7: note = Synthetic
GNTTKST

SEQ ID NO: 233 - protein, 7 aa, synthetic construct
  REGION 1..7: note = Synthetic
MNHIRTT

SEQ ID NO: 234 - protein, 7 aa, synthetic construct
  REGION 1..7: note = Synthetic
SNQTRSV

SEQ ID NO: 235 - protein, 7 aa, synthetic construct
  REGION 1..7: note = Synthetic
DNSTRAT

SEQ ID NO: 236 - protein, 7 aa, synthetic construct
  REGION 1..7: note = Synthetic
GNTVKDP

SEQ ID NO: 237 - protein, 7 aa, synthetic construct
  REGION 1..7: note = Synthetic
MNNARPY

SEQ ID NO: 238 - protein, 7 aa, synthetic construct
  REGION 1..7: note = Synthetic
SNSTKIL

SEQ ID NO: 239 - protein, 7 aa, synthetic construct
  REGION 1..7: note = Synthetic
DNSTRWS

SEQ ID NO: 240 - protein, 7 aa, synthetic construct
  REGION 1..7: note = Synthetic
GNTVKLL

SEQ ID NO: 241 - protein, 7 aa, synthetic construct
  REGION 1..7: note = Synthetic
MNQARNP

SEQ ID NO: 242 - protein, 7 aa, synthetic construct
  REGION 1..7: note = Synthetic
SNSTRSV

SEQ ID NO: 243 - protein, 7 aa, synthetic construct
  REGION 1..7: note = Synthetic
DNSVRLT

SEQ ID NO: 244 - protein, 7 aa, synthetic construct
  REGION 1..7: note = Synthetic
GNTVRDT

SEQ ID NO: 245 - protein, 7 aa, synthetic construct
  REGION 1..7: note = Synthetic
MNTIRSY

SEQ ID NO: 246 - protein, 7 aa, synthetic construct
  REGION 1..7: note = Synthetic
SNSVRAM

SEQ ID NO: 247 - protein, 7 aa, synthetic construct
  REGION 1..7: note = Synthetic
DNTVRGS

SEQ ID NO: 248 - protein, 7 aa, synthetic construct
  REGION 1..7: note = Synthetic
GNVIKTW

SEQ ID NO: 249 - protein, 7 aa, synthetic construct
  REGION 1..7: note = Synthetic
MNTVRDY

SEQ ID NO: 250 - protein, 7 aa, synthetic construct
  REGION 1..7: note = Synthetic
SNSVRDR

SEQ ID NO: 251 - protein, 7 aa, synthetic construct
  REGION 1..7: note = Synthetic
DNTWKAA

SEQ ID NO: 252 - protein, 7 aa, synthetic construct
  REGION 1..7: note = Synthetic
GNVIRSH

SEQ ID NO: 253 - protein, 7 aa, synthetic construct
  REGION 1..7: note = Synthetic
NNALKPY

SEQ ID NO: 254 - protein, 7 aa, synthetic construct
  REGION 1..7: note = Synthetic
SNTIKNL

SEQ ID NO: 255 - protein, 7 aa, synthetic construct
  REGION 1..7: note = Synthetic
ENATRSL

SEQ ID NO: 256 - protein, 7 aa, synthetic construct
  REGION 1..7: note = Synthetic
GNVTRST

SEQ ID NO: 257 - protein, 7 aa, synthetic construct
  REGION 1..7: note = Synthetic
NNASKGW

SEQ ID NO: 258 - protein, 7 aa, synthetic construct
  REGION 1..7: note = Synthetic
SNTIRTI

SEQ ID NO: 259 - protein, 7 aa, synthetic construct
  REGION 1..7: note = Synthetic
ENHLRNT

SEQ ID NO: 260 - protein, 7 aa, synthetic construct
  REGION 1..7: note = Synthetic
GNYGRAQ

SEQ ID NO: 261 - protein, 7 aa, synthetic construct
  REGION 1..7: note = Synthetic
NNATRNG

SEQ ID NO: 262 - protein, 7 aa, synthetic construct
  REGION 1..7: note = Synthetic
SNTTKAG

SEQ ID NO: 263 - protein, 7 aa, synthetic construct
  REGION 1..7: note = Synthetic
ENHVRNM

SEQ ID NO: 264 - protein, 7 aa, synthetic construct
  REGION 1..7: note = Synthetic
HNMSRIA

SEQ ID NO: 265 - protein, 7 aa, synthetic construct
  REGION 1..7: note = Synthetic
NNFTRGT

SEQ ID NO: 266 - protein, 7 aa, synthetic construct
  REGION 1..7: note = Synthetic
TNAVRGT

SEQ ID NO: 267 - protein, 7 aa, synthetic construct
  REGION 1..7: note = Synthetic
ENNTKIT

SEQ ID NO: 268 - protein, 7 aa, synthetic construct
  REGION 1..7: note = Synthetic
INDARTV

SEQ ID NO: 269 - protein, 7 aa, synthetic construct
  REGION 1..7: note = Synthetic
NNNGRSL

SEQ ID NO: 270 - protein, 7 aa, synthetic construct
  REGION 1..7: note = Synthetic
TNELRSY

SEQ ID NO: 271 - protein, 7 aa, synthetic construct
  REGION 1..7: note = Synthetic
ENPIRGR

SEQ ID NO: 272 - protein, 7 aa, synthetic construct
  REGION 1..7: note = Synthetic
INESRNR

SEQ ID NO: 273 - protein, 7 aa, synthetic construct
  REGION 1..7: note = Synthetic
NNSARPL

SEQ ID NO: 274 - protein, 7 aa, synthetic construct
  REGION 1..7: note = Synthetic
TNEVRLS

SEQ ID NO: 275 - protein, 7 aa, synthetic construct
  REGION 1..7: note = Synthetic
ENRIRDN

SEQ ID NO: 276 - protein, 7 aa, synthetic construct
  REGION 1..7: note = Synthetic
INHLKSA

SEQ ID NO: 277 - protein, 7 aa, synthetic construct
  REGION 1..7: note = Synthetic
NNSTRAG

SEQ ID NO: 278 - protein, 7 aa, synthetic construct
  REGION 1..7: note = Synthetic
TNFTKMT

SEQ ID NO: 279 - protein, 7 aa, synthetic construct
  REGION 1..7: note = Synthetic
ENSIKPL

SEQ ID NO: 280 - protein, 7 aa, synthetic construct
  REGION 1..7: note = Synthetic
INNERSK

SEQ ID NO: 281 - protein, 7 aa, synthetic construct
  REGION 1..7: note = Synthetic
NNSTRGG

SEQ ID NO: 282 - protein, 7 aa, synthetic construct
  REGION 1..7: note = Synthetic
TNGTKPT

SEQ ID NO: 283 - protein, 7 aa, synthetic construct
  REGION 1..7: note = Synthetic
ENSVRSA

SEQ ID NO: 284 - protein, 7 aa, synthetic construct
  REGION 1..7: note = Synthetic
INNVKSA

SEQ ID NO: 285 - protein, 7 aa, synthetic construct
  REGION 1..7: note = Synthetic
NNSTRGL

SEQ ID NO: 286 - protein, 7 aa, synthetic construct
  REGION 1..7: note = Synthetic
TNGTRNL

SEQ ID NO: 287 - protein, 7 aa, synthetic construct
  REGION 1..7: note = Synthetic
ENVVRSK

SEQ ID NO: 288 - protein, 7 aa, synthetic construct
  REGION 1..7: note = Synthetic
INNVRSV

SEQ ID NO: 289 - protein, 7 aa, synthetic construct
  REGION 1..7: note = Synthetic
NNTIRYS

SEQ ID NO: 290 - protein, 7 aa, synthetic construct
  REGION 1..7: note = Synthetic
TNHIRFT

SEQ ID NO: 291 - protein, 7 aa, synthetic construct
  REGION 1..7: note = Synthetic
GNATKAT

SEQ ID NO: 292 - protein, 7 aa, synthetic construct
  REGION 1..7: note = Synthetic
INPRKDN

SEQ ID NO: 293 - protein, 7 aa, synthetic construct
  REGION 1..7: note = Synthetic
NNTTRGM

SEQ ID NO: 294 - protein, 7 aa, synthetic construct
  REGION 1..7: note = Synthetic
TNHIRHL

SEQ ID NO: 295 - protein, 7 aa, synthetic construct
  REGION 1..7: note = Synthetic
GNAVKST

SEQ ID NO: 296 - protein, 7 aa, synthetic construct
  REGION 1..7: note = Synthetic
INQIRAA

SEQ ID NO: 297 - protein, 7 aa, synthetic construct
  REGION 1..7: note = Synthetic
NNTVRGM

SEQ ID NO: 298 - protein, 7 aa, synthetic construct
  REGION 1..7: note = Synthetic
TNHIRNL

SEQ ID NO: 299 - protein, 7 aa, synthetic construct
  REGION 1..7: note = Synthetic
GNAVRTG

SEQ ID NO: 300 - protein, 7 aa, synthetic construct
  REGION 1..7: note = Synthetic
INQIRQV

SEQ ID NO: 301 - protein, 7 aa, synthetic construct
  REGION 1..7: note = Synthetic
NNVIRGF

SEQ ID NO: 302 - protein, 7 aa, synthetic construct
  REGION 1..7: note = Synthetic
TNHSRPV

SEQ ID NO: 303 - protein, 7 aa, synthetic construct
  REGION 1..7: note = Synthetic
GNAVRTV

SEQ ID NO: 304 - protein, 7 aa, synthetic construct
  REGION 1..7: note = Synthetic
INSIRDL

SEQ ID NO: 305 - protein, 7 aa, synthetic construct
  REGION 1..7: note = Synthetic
PNDIRLR

SEQ ID NO: 306 - protein, 7 aa, synthetic construct
  REGION 1..7: note = Synthetic
TNMIRNA

SEQ ID NO: 307 - protein, 7 aa, synthetic construct
  REGION 1..7: note = Synthetic
GNDIRVR

SEQ ID NO: 308 - protein, 7 aa, synthetic construct
  REGION 1..7: note = Synthetic
INSIRMT

SEQ ID NO: 309 - protein, 7 aa, synthetic construct
  REGION 1..7: note = Synthetic
PNGIKGV

SEQ ID NO: 310 - protein, 7 aa, synthetic construct
  REGION 1..7: note = Synthetic
TNMPRNS

SEQ ID NO: 311 - protein, 7 aa, synthetic construct
  REGION 1..7: note = Synthetic
GNDIRYY

SEQ ID NO: 312 - protein, 7 aa, synthetic construct
  REGION 1..7: note = Synthetic
INTVRDR

SEQ ID NO: 313 - protein, 7 aa, synthetic construct
  REGION 1..7: note = Synthetic
PNNLRTP

SEQ ID NO: 314 - protein, 7 aa, synthetic construct
  REGION 1..7: note = Synthetic
TNPSRFA

SEQ ID NO: 315 - protein, 7 aa, synthetic construct
  REGION 1..7: note = Synthetic
GNDTRST

SEQ ID NO: 316 - protein, 7 aa, synthetic construct
  REGION 1..7: note = Synthetic
INYQKPA

SEQ ID NO: 317 - protein, 7 aa, synthetic construct
  REGION 1..7: note = Synthetic
PNNVRQY

SEQ ID NO: 318 - protein, 7 aa, synthetic construct
  REGION 1..7: note = Synthetic
TNSIRDQ

SEQ ID NO: 319 - protein, 7 aa, synthetic construct
  REGION 1..7: note = Synthetic
GNESRPT

SEQ ID NO: 320 - protein, 7 aa, synthetic construct
  REGION 1..7: note = Synthetic
INYVKHH

SEQ ID NO: 321 - protein, 7 aa, synthetic construct
  REGION 1..7: note = Synthetic
PNSIRRD

SEQ ID NO: 322 - protein, 7 aa, synthetic construct
  REGION 1..7: note = Synthetic
TNSTKAG

SEQ ID NO: 323 - protein, 7 aa, synthetic construct
  REGION 1..7: note = Synthetic
GNESRYM

SEQ ID NO: 324 - protein, 7 aa, synthetic construct
  REGION 1..7: note = Synthetic
KNLLRSE

SEQ ID NO: 325 - protein, 7 aa, synthetic construct
  REGION 1..7: note = Synthetic
PNSMRQV

SEQ ID NO: 326 - protein, 7 aa, synthetic construct
  REGION 1..7: note = Synthetic
TNSTRFT

SEQ ID NO: 327 - protein, 7 aa, synthetic construct
  REGION 1..7: note = Synthetic
GNEVRYY

SEQ ID NO: 328 - protein, 7 aa, synthetic construct
  REGION 1..7: note = Synthetic
LNDLRSR

SEQ ID NO: 329 - protein, 7 aa, synthetic construct
  REGION 1..7: note = Synthetic
PNSTRNV

SEQ ID NO: 330 - protein, 7 aa, synthetic construct
  REGION 1..7: note = Synthetic
TNSTRGV

SEQ ID NO: 331 - protein, 7 aa, synthetic construct
  REGION 1..7: note = Synthetic
GNFTRDL

SEQ ID NO: 332 - protein, 7 aa, synthetic construct
  REGION 1..7: note = Synthetic
LNEIRAV

SEQ ID NO: 333 - protein, 7 aa, synthetic construct
  REGION 1..7: note = Synthetic
PNTIRNV

SEQ ID NO: 334 - protein, 7 aa, synthetic construct
  REGION 1..7: note = Synthetic
TNSVRPV

SEQ ID NO: 335 - protein, 7 aa, synthetic construct
  REGION 1..7: note = Synthetic
GNGTRVY

SEQ ID NO: 336 - protein, 7 aa, synthetic construct
  REGION 1..7: note = Synthetic
LNEPRRV

SEQ ID NO: 337 - protein, 7 aa, synthetic construct
  REGION 1..7: note = Synthetic
PNTTRYL

SEQ ID NO: 338 - protein, 7 aa, synthetic construct
  REGION 1..7: note = Synthetic
TNTVRGG

SEQ ID NO: 339 - protein, 7 aa, synthetic construct
  REGION 1..7: note = Synthetic
GNHVRDA

SEQ ID NO: 340 - protein, 7 aa, synthetic construct
  REGION 1..7: note = Synthetic
LNEVKLY

SEQ ID NO: 341 - protein, 7 aa, synthetic construct
  REGION 1..7: note = Synthetic
QNDVRYP

SEQ ID NO: 342 - protein, 7 aa, synthetic construct
  REGION 1..7: note = Synthetic
TNVVKQT

SEQ ID NO: 343 - protein, 7 aa, synthetic construct
  REGION 1..7: note = Synthetic
GNLGRSS

SEQ ID NO: 344 - protein, 7 aa, synthetic construct
  REGION 1..7: note = Synthetic
LNHMRNT

SEQ ID NO: 345 - protein, 7 aa, synthetic construct
  REGION 1..7: note = Synthetic
QNEIKTY

SEQ ID NO: 346 - protein, 7 aa, synthetic construct
  REGION 1..7: note = Synthetic
TNYTKTL

SEQ ID NO: 347 - protein, 7 aa, synthetic construct
  REGION 1..7: note = Synthetic
GNLTKGY

SEQ ID NO: 348 - protein, 7 aa, synthetic construct
  REGION 1..7: note = Synthetic
LNNIRNV

SEQ ID NO: 349 - protein, 7 aa, synthetic construct
  REGION 1..7: note = Synthetic
QNKHREM

SEQ ID NO: 350 - protein, 7 aa, synthetic construct
  REGION 1..7: note = Synthetic
VNATRGG

SEQ ID NO: 351 - protein, 7 aa, synthetic construct
  REGION 1..7: note = Synthetic
GNMIRND

SEQ ID NO: 352 - protein, 7 aa, synthetic construct
  REGION 1..7: note = Synthetic
LNNIRQV

SEQ ID NO: 353 - protein, 7 aa, synthetic construct
  REGION 1..7: note = Synthetic
QNNIKAW

SEQ ID NO: 354 - protein, 7 aa, synthetic construct
  REGION 1..7: note = Synthetic
VNATRST

SEQ ID NO: 355 - protein, 7 aa, synthetic construct
  REGION 1..7: note = Synthetic
GNNTKAV

SEQ ID NO: 356 - protein, 7 aa, synthetic construct
  REGION 1..7: note = Synthetic
LNNVRPT

SEQ ID NO: 357 - protein, 7 aa, synthetic construct
  REGION 1..7: note = Synthetic
QNRMRND

SEQ ID NO: 358 - protein, 7 aa, synthetic construct
  REGION 1..7: note = Synthetic
VNEVRFQ

SEQ ID NO: 359 - protein, 7 aa, synthetic construct
  REGION 1..7: note = Synthetic
GNNTRSA

SEQ ID NO: 360 - protein, 7 aa, synthetic construct
  REGION 1..7: note = Synthetic
LNNVRSP

SEQ ID NO: 361 - protein, 7 aa, synthetic construct
  REGION 1..7: note = Synthetic
QNRQRDT

SEQ ID NO: 362 - protein, 7 aa, synthetic construct
  REGION 1..7: note = Synthetic
VNHDRAR

SEQ ID NO: 363 - protein, 7 aa, synthetic construct
  REGION 1..7: note = Synthetic
GNNVKGL

SEQ ID NO: 364 - protein, 7 aa, synthetic construct
  REGION 1..7: note = Synthetic
LNNVRSV

SEQ ID NO: 365 - protein, 7 aa, synthetic construct
  REGION 1..7: note = Synthetic
RNDGRVA

SEQ ID NO: 366 - protein, 7 aa, synthetic construct
  REGION 1..7: note = Synthetic
VNHIRLQ

SEQ ID NO: 367 - protein, 7 aa, synthetic construct
  REGION 1..7: note = Synthetic
GNNVKQF

SEQ ID NO: 368 - protein, 7 aa, synthetic construct
  REGION 1..7: note = Synthetic
LNQYRAA

SEQ ID NO: 369 - protein, 7 aa, synthetic construct
  REGION 1..7: note = Synthetic
RNDTRHI

SEQ ID NO: 370 - protein, 7 aa, synthetic construct
  REGION 1..7: note = Synthetic
VNHLREV

SEQ ID NO: 371 - protein, 7 aa, synthetic construct
  REGION 1..7: note = Synthetic
GNQTKPF

SEQ ID NO: 372 - protein, 7 aa, synthetic construct
  REGION 1..7: note = Synthetic
LNRVRGD

SEQ ID NO: 373 - protein, 7 aa, synthetic construct
  REGION 1..7: note = Synthetic
RNERRDV

SEQ ID NO: 374 - protein, 7 aa, synthetic construct
  REGION 1..7: note = Synthetic
VNHVRLT

SEQ ID NO: 375 - protein, 7 aa, synthetic construct
  REGION 1..7: note = Synthetic
GNRVKET

SEQ ID NO: 376 - protein, 7 aa, synthetic construct
  REGION 1..7: note = Synthetic
LNSARSI

SEQ ID NO: 377 - protein, 7 aa, synthetic construct
  REGION 1..7: note = Synthetic
RNEVRFA

SEQ ID NO: 378 - protein, 7 aa, synthetic construct
  REGION 1..7: note = Synthetic
VNNERSK

SEQ ID NO: 379 - protein, 7 aa, synthetic construct
  REGION 1..7: note = Synthetic
GNSARNI

SEQ ID NO: 380 - protein, 7 aa, synthetic construct
  REGION 1..7: note = Synthetic
LNSGRSA

SEQ ID NO: 381 - protein, 7 aa, synthetic construct
  REGION 1..7: note = Synthetic
RNTVKDP

SEQ ID NO: 382 - protein, 7 aa, synthetic construct
  REGION 1..7: note = Synthetic
VNTIRNV

SEQ ID NO: 383 - protein, 7 aa, synthetic construct
  REGION 1..7: note = Synthetic
GNSIRAL

SEQ ID NO: 384 - protein, 7 aa, synthetic construct
  REGION 1..7: note = Synthetic
LNSPRNV

SEQ ID NO: 385 - protein, 7 aa, synthetic construct
  REGION 1..7: note = Synthetic
SNAIKAT

SEQ ID NO: 386 - protein, 7 aa, synthetic construct
  REGION 1..7: note = Synthetic
VNTIRSV

SEQ ID NO: 387 - protein, 7 aa, synthetic construct
  REGION 1..7: note = Synthetic
YNQMRNT

SEQ ID NO: 388 - protein, 7 aa, synthetic construct
  REGION 1..7: note = Synthetic
ANAERQP

SEQ ID NO: 389 - protein, 7 aa, synthetic construct
  REGION 1..7: note = Synthetic
GNETKSV

SEQ ID NO: 390 - protein, 7 aa, synthetic construct
  REGION 1..7: note = Synthetic
NNMVRTP

SEQ ID NO: 391 - protein, 7 aa, synthetic construct
  REGION 1..7: note = Synthetic
VNTVREF

SEQ ID NO: 392 - protein, 7 aa, synthetic construct
  REGION 1..7: note = Synthetic
ANDMRSG

SEQ ID NO: 393 - protein, 7 aa, synthetic construct
  REGION 1..7: note = Synthetic
GNETRML

SEQ ID NO: 394 - protein, 7 aa, synthetic construct
  REGION 1..7: note = Synthetic
NNSIKVG

SEQ ID NO: 395 - protein, 7 aa, synthetic construct
  REGION 1..7: note = Synthetic
VNTVREF

SEQ ID NO: 396 - protein, 7 aa, synthetic construct
  REGION 1..7: note = Synthetic
ANDSRAT

SEQ ID NO: 397 - protein, 7 aa, synthetic construct
  REGION 1..7: note = Synthetic
GNGTRLN

SEQ ID NO: 398 - protein, 7 aa, synthetic construct
  REGION 1..7: note = Synthetic
NNSLKEK

SEQ ID NO: 399 - protein, 7 aa, synthetic construct
  REGION 1..7: note = Synthetic
WNANRIN

SEQ ID NO: 400 - protein, 7 aa, synthetic construct
  REGION 1..7: note = Synthetic
ANGTKII

SEQ ID NO: 401 - protein, 7 aa, synthetic construct
  REGION 1..7: note = Synthetic
GNHVKQD

SEQ ID NO: 402 - protein, 7 aa, synthetic construct
  REGION 1..7: note = Synthetic
NNTVKNW

SEQ ID NO: 403 - protein, 7 aa, synthetic construct
  REGION 1..7: note = Synthetic
WNENKSM

SEQ ID NO: 404 - protein, 7 aa, synthetic construct
  REGION 1..7: note = Synthetic
ANITKSI

SEQ ID NO: 405 - protein, 7 aa, synthetic construct
  REGION 1..7: note = Synthetic
GNISKNI

SEQ ID NO: 406 - protein, 7 aa, synthetic construct
  REGION 1..7: note = Synthetic
NNVSRDS

SEQ ID NO: 407 - protein, 7 aa, synthetic construct
  REGION 1..7: note = Synthetic
YNAAKGV

SEQ ID NO: 408 - protein, 7 aa, synthetic construct
  REGION 1..7: note = Synthetic
ANLSKTT

SEQ ID NO: 409 - protein, 7 aa, synthetic construct
  REGION 1..7: note = Synthetic
GNLVKEN

SEQ ID NO: 410 - protein, 7 aa, synthetic construct
  REGION 1..7: note = Synthetic
NNVSRNN

SEQ ID NO: 411 - protein, 7 aa, synthetic construct
  REGION 1..7: note = Synthetic
YNDDRVF

SEQ ID NO: 412 - protein, 7 aa, synthetic construct
  REGION 1..7: note = Synthetic
DNAHRTQ

SEQ ID NO: 413 - protein, 7 aa, synthetic construct
  REGION 1..7: note = Synthetic
GNNIRNY

SEQ ID NO: 414 - protein, 7 aa, synthetic construct
  REGION 1..7: note = Synthetic
PNASRDW

SEQ ID NO: 415 - protein, 7 aa, synthetic construct
  REGION 1..7: note = Synthetic
YNERKEV

SEQ ID NO: 416 - protein, 7 aa, synthetic construct
  REGION 1..7: note = Synthetic
DNDIRGK

SEQ ID NO: 417 - protein, 7 aa, synthetic construct
  REGION 1..7: note = Synthetic
GNSARAV

SEQ ID NO: 418 - protein, 7 aa, synthetic construct
  REGION 1..7: note = Synthetic
PNNNKNH

SEQ ID NO: 419 - protein, 7 aa, synthetic construct
  REGION 1..7: note = Synthetic
YNESRNL

SEQ ID NO: 420 - protein, 7 aa, synthetic construct
  REGION 1..7: note = Synthetic
DNESKKS

SEQ ID NO: 421 - protein, 7 aa, synthetic construct
  REGION 1..7: note = Synthetic
GNVVKHM

SEQ ID NO: 422 - protein, 7 aa, synthetic construct
  REGION 1..7: note = Synthetic
PNTSKFT

SEQ ID NO: 423 - protein, 7 aa, synthetic construct
  REGION 1..7: note = Synthetic
YNEVKAN

SEQ ID NO: 424 - protein, 7 aa, synthetic construct
  REGION 1..7: note = Synthetic
DNKTRLT

SEQ ID NO: 425 - protein, 7 aa, synthetic construct
  REGION 1..7: note = Synthetic
GNYVRDH

SEQ ID NO: 426 - protein, 7 aa, synthetic construct
  REGION 1..7: note = Synthetic
QNALRST

SEQ ID NO: 427 - protein, 7 aa, synthetic construct
  REGION 1..7: note = Synthetic
YNHQKHD

SEQ ID NO: 428 - protein, 7 aa, synthetic construct
  REGION 1..7: note = Synthetic
DNNGRLT

SEQ ID NO: 429 - protein, 7 aa, synthetic construct
  REGION 1..7: note = Synthetic
HNLDRSN

SEQ ID NO: 430 - protein, 7 aa, synthetic construct
  REGION 1..7: note = Synthetic
QNALRST

SEQ ID NO: 431 - protein, 7 aa, synthetic construct
  REGION 1..7: note = Synthetic
YNLSRDD

SEQ ID NO: 432 - protein, 7 aa, synthetic construct
  REGION 1..7: note = Synthetic
DNNVRNV

SEQ ID NO: 433 - protein, 7 aa, synthetic construct
  REGION 1..7: note = Synthetic
HNLDRSN

SEQ ID NO: 434 - protein, 7 aa, synthetic construct
  REGION 1..7: note = Synthetic
QNGDKLP

SEQ ID NO: 435 - protein, 7 aa, synthetic construct
  REGION 1..7: note = Synthetic
YNMGKDH

SEQ ID NO: 436 - protein, 7 aa, synthetic construct
  REGION 1..7: note = Synthetic
DNPSRGI

SEQ ID NO: 437 - protein, 7 aa, synthetic construct
  REGION 1..7: note = Synthetic
HNLERQT

SEQ ID NO: 438 - protein, 7 aa, synthetic construct
  REGION 1..7: note = Synthetic
QNNIKDK

SEQ ID NO: 439 - protein, 7 aa, synthetic construct
  REGION 1..7: note = Synthetic
YNSIRNN

SEQ ID NO: 440 - protein, 7 aa, synthetic construct
  REGION 1..7: note = Synthetic
DNPVRGI

SEQ ID NO: 441 - protein, 7 aa, synthetic construct
  REGION 1..7: note = Synthetic
HNLERQT

SEQ ID NO: 442 - protein, 7 aa, synthetic construct
  REGION 1..7: note = Synthetic
QNVARNS

SEQ ID NO: 443 - protein, 7 aa, synthetic construct
  REGION 1..7: note = Synthetic
YNSNKPN

SEQ ID NO: 444 - protein, 7 aa, synthetic construct
  REGION 1..7: note = Synthetic
DNRHKEG

SEQ ID NO: 445 - protein, 7 aa, synthetic construct
  REGION 1..7: note = Synthetic
HNLHREN

SEQ ID NO: 446 - protein, 7 aa, synthetic construct
  REGION 1..7: note = Synthetic
RNADKGI

SEQ ID NO: 447 - protein, 7 aa, synthetic construct
  REGION 1..7: note = Synthetic
YNTQKYG

SEQ ID NO: 448 - protein, 7 aa, synthetic construct
  REGION 1..7: note = Synthetic
DNRIRGD

SEQ ID NO: 449 - protein, 7 aa, synthetic construct
  REGION 1..7: note = Synthetic
INETRNL

SEQ ID NO: 450 - protein, 7 aa, synthetic construct
  REGION 1..7: note = Synthetic
RNDARNS

SEQ ID NO: 451 - protein, 7 aa, synthetic construct
  REGION 1..7: note = Synthetic
DNSARET

SEQ ID NO: 452 - protein, 7 aa, synthetic construct
  REGION 1..7: note = Synthetic
INGDRAR

SEQ ID NO: 453 - protein, 7 aa, synthetic construct
  REGION 1..7: note = Synthetic
RNGSKSD

SEQ ID NO: 454 - protein, 7 aa, synthetic construct
  REGION 1..7: note = Synthetic
ENASKAF

SEQ ID NO: 455 - protein, 7 aa, synthetic construct
  REGION 1..7: note = Synthetic
INGSRNL

SEQ ID NO: 456 - protein, 7 aa, synthetic construct
  REGION 1..7: note = Synthetic
RNNVKAD

SEQ ID NO: 457 - protein, 7 aa, synthetic construct
  REGION 1..7: note = Synthetic
ENAVRSG

SEQ ID NO: 458 - protein, 7 aa, synthetic construct
  REGION 1..7: note = Synthetic
INKEKTI

SEQ ID NO: 459 - protein, 7 aa, synthetic construct
  REGION 1..7: note = Synthetic
RNTEKEQ

SEQ ID NO: 460 - protein, 7 aa, synthetic construct
  REGION 1..7: note = Synthetic
ENHLRNT

SEQ ID NO: 461 - protein, 7 aa, synthetic construct
  REGION 1..7: note = Synthetic
INMSKSA

SEQ ID NO: 462 - protein, 7 aa, synthetic construct
  REGION 1..7: note = Synthetic
SNDGRSN

SEQ ID NO: 463 - protein, 7 aa, synthetic construct
  REGION 1..7: note = Synthetic
ENHTRDK

SEQ ID NO: 464 - protein, 7 aa, synthetic construct
  REGION 1..7: note = Synthetic
INMSKSA

SEQ ID NO: 465 - protein, 7 aa, synthetic construct
  REGION 1..7: note = Synthetic
SNDQKDN

SEQ ID NO: 466 - protein, 7 aa, synthetic construct
  REGION 1..7: note = Synthetic
ENHVKQN

SEQ ID NO: 467 - protein, 7 aa, synthetic construct
  REGION 1..7: note = Synthetic
INSDRGD

SEQ ID NO: 468 - protein, 7 aa, synthetic construct
  REGION 1..7: note = Synthetic
SNEARSL

SEQ ID NO: 469 - protein, 7 aa, synthetic construct
  REGION 1..7: note = Synthetic
ENITKNV

SEQ ID NO: 470 - protein, 7 aa, synthetic construct
  REGION 1..7: note = Synthetic
KNTSRED

SEQ ID NO: 471 - protein, 7 aa, synthetic construct
  REGION 1..7: note = Synthetic
SNEPRVL

SEQ ID NO: 472 - protein, 7 aa, synthetic construct
  REGION 1..7: note = Synthetic
ENLERMV

SEQ ID NO: 473 - protein, 7 aa, synthetic construct
  REGION 1..7: note = Synthetic
LNATRNL

SEQ ID NO: 474 - protein, 7 aa, synthetic construct
  REGION 1..7: note = Synthetic
SNGDRSR

SEQ ID NO: 475 - protein, 7 aa, synthetic construct
  REGION 1..7: note = Synthetic
ENLIKQH

SEQ ID NO: 476 - protein, 7 aa, synthetic construct
  REGION 1..7: note = Synthetic
LNDIRNT

SEQ ID NO: 477 - protein, 7 aa, synthetic construct
  REGION 1..7: note = Synthetic
SNGGRLQ

SEQ ID NO: 478 - protein, 7 aa, synthetic construct
  REGION 1..7: note = Synthetic
ENLIRSN

SEQ ID NO: 479 - protein, 7 aa, synthetic construct
  REGION 1..7: note = Synthetic
LNDTRYY

SEQ ID NO: 480 - protein, 7 aa, synthetic construct
  REGION 1..7: note = Synthetic
SNGHKLS

SEQ ID NO: 481 - protein, 7 aa, synthetic construct
  REGION 1..7: note = Synthetic
ENLIRSN

SEQ ID NO: 482 - protein, 7 aa, synthetic construct
  REGION 1..7: note = Synthetic
LNGARIV

SEQ ID NO: 483 - protein, 7 aa, synthetic construct
  REGION 1..7: note = Synthetic
SNMNRIT

SEQ ID NO: 484 - protein, 7 aa, synthetic construct
  REGION 1..7: note = Synthetic
ENLLRSS

SEQ ID NO: 485 - protein, 7 aa, synthetic construct
  REGION 1..7: note = Synthetic
LNGARTD

SEQ ID NO: 486 - protein, 7 aa, synthetic construct
  REGION 1..7: note = Synthetic
SNMPRDT

SEQ ID NO: 487 - protein, 7 aa, synthetic construct
  REGION 1..7: note = Synthetic
ENLMRSS

SEQ ID NO: 488 - protein, 7 aa, synthetic construct
  REGION 1..7: note = Synthetic
LNGLRAH

SEQ ID NO: 489 - protein, 7 aa, synthetic construct
  REGION 1..7: note = Synthetic
SNNIRPI

SEQ ID NO: 490 - protein, 7 aa, synthetic construct
  REGION 1..7: note = Synthetic
ENLVKDD

SEQ ID NO: 491 - protein, 7 aa, synthetic construct
  REGION 1..7: note = Synthetic
LNGMKGA

SEQ ID NO: 492 - protein, 7 aa, synthetic construct
  REGION 1..7: note = Synthetic
SNNMRPA

SEQ ID NO: 493 - protein, 7 aa, synthetic construct
  REGION 1..7: note = Synthetic
ENNPRAT

SEQ ID NO: 494 - protein, 7 aa, synthetic construct
  REGION 1..7: note = Synthetic
LNGNRYS

SEQ ID NO: 495 - protein, 7 aa, synthetic construct
  REGION 1..7: note = Synthetic
SNNTKNF

SEQ ID NO: 496 - protein, 7 aa, synthetic construct
  REGION 1..7: note = Synthetic
ENNTKNY

SEQ ID NO: 497 - protein, 7 aa, synthetic construct
  REGION 1..7: note = Synthetic
LNGSRGA

SEQ ID NO: 498 - protein, 7 aa, synthetic construct
  REGION 1..7: note = Synthetic
SNPDRMK

SEQ ID NO: 499 - protein, 7 aa, synthetic construct
  REGION 1..7: note = Synthetic
ENQHRTF

SEQ ID NO: 500 - protein, 7 aa, synthetic construct
  REGION 1..7: note = Synthetic
LNGTRSL

SEQ ID NO: 501 - protein, 7 aa, synthetic construct
  REGION 1..7: note = Synthetic
SNTIKNL

SEQ ID NO: 502 - protein, 7 aa, synthetic construct
  REGION 1..7: note = Synthetic
ENTIRNT

SEQ ID NO: 503 - protein, 7 aa, synthetic construct
  REGION 1..7: note = Synthetic
LNLIKNN

SEQ ID NO: 504 - protein, 7 aa, synthetic construct
  REGION 1..7: note = Synthetic
TNAVKFT

SEQ ID NO: 505 - protein, 7 aa, synthetic construct
  REGION 1..7: note = Synthetic
ENTIRNT

SEQ ID NO: 506 - protein, 7 aa, synthetic construct
  REGION 1..7: note = Synthetic
LNMERTT

SEQ ID NO: 507 - protein, 7 aa, synthetic construct
  REGION 1..7: note = Synthetic
TNAVKTQ

SEQ ID NO: 508 - protein, 7 aa, synthetic construct
  REGION 1..7: note = Synthetic
ENTMKAH

SEQ ID NO: 509 - protein, 7 aa, synthetic construct
  REGION 1..7: note = Synthetic
LNNIKNT

SEQ ID NO: 510 - protein, 7 aa, synthetic construct
  REGION 1..7: note = Synthetic
TNDHKQY

SEQ ID NO: 511 - protein, 7 aa, synthetic construct
  REGION 1..7: note = Synthetic
ENYIRDK

SEQ ID NO: 512 - protein, 7 aa, synthetic construct
  REGION 1..7: note = Synthetic
LNSERSY

SEQ ID NO: 513 - protein, 7 aa, synthetic construct
  REGION 1..7: note = Synthetic
TNGGKYL

SEQ ID NO: 514 - protein, 7 aa, synthetic construct
  REGION 1..7: note = Synthetic
ENYIRDK

SEQ ID NO: 515 - protein, 7 aa, synthetic construct
  REGION 1..7: note = Synthetic
LNSLRGE

SEQ ID NO: 516 - protein, 7 aa, synthetic construct
  REGION 1..7: note = Synthetic
TNGIRNM

SEQ ID NO: 517 - protein, 7 aa, synthetic construct
  REGION 1..7: note = Synthetic
ENYVRER

SEQ ID NO: 518 - protein, 7 aa, synthetic construct
  REGION 1..7: note = Synthetic
LNVDRLI

SEQ ID NO: 519 - protein, 7 aa, synthetic construct
  REGION 1..7: note = Synthetic
TNGIRPW

SEQ ID NO: 520 - protein, 7 aa, synthetic construct
  REGION 1..7: note = Synthetic
FNDEKHT

SEQ ID NO: 521 - protein, 7 aa, synthetic construct
  REGION 1..7: note = Synthetic
MNGGKSL

SEQ ID NO: 522 - protein, 7 aa, synthetic construct
  REGION 1..7: note = Synthetic
TNGMRNQ

SEQ ID NO: 523 - protein, 7 aa, synthetic construct
  REGION 1..7: note = Synthetic
FNDGRTN

SEQ ID NO: 524 - protein, 7 aa, synthetic construct
  REGION 1..7: note = Synthetic
MNGTKAL

SEQ ID NO: 525 - protein, 7 aa, synthetic construct
  REGION 1..7: note = Synthetic
TNGVRNS

SEQ ID NO: 526 - protein, 7 aa, synthetic construct
  REGION 1..7: note = Synthetic
FNDNKAF

SEQ ID NO: 527 - protein, 7 aa, synthetic construct
  REGION 1..7: note = Synthetic
MNHSKSA

SEQ ID NO: 528 - protein, 7 aa, synthetic construct
  REGION 1..7: note = Synthetic
TNKERFN

SEQ ID NO: 529 - protein, 7 aa, synthetic construct
  REGION 1..7: note = Synthetic
FNGNKMH

SEQ ID NO: 530 - protein, 7 aa, synthetic construct
  REGION 1..7: note = Synthetic
MNMDRNT

SEQ ID NO: 531 - protein, 7 aa, synthetic construct
  REGION 1..7: note = Synthetic
TNMVKDW

SEQ ID NO: 532 - protein, 7 aa, synthetic construct
  REGION 1..7: note = Synthetic
FNGTRNT

SEQ ID NO: 533 - protein, 7 aa, synthetic construct
  REGION 1..7: note = Synthetic
MNSIRNT

SEQ ID NO: 534 - protein, 7 aa, synthetic construct
  REGION 1..7: note = Synthetic
TNQTKSI

SEQ ID NO: 535 - protein, 7 aa, synthetic construct
  REGION 1..7: note = Synthetic
FNNLKID

SEQ ID NO: 536 - protein, 7 aa, synthetic construct
  REGION 1..7: note = Synthetic
MNSLRSD

SEQ ID NO: 537 - protein, 7 aa, synthetic construct
  REGION 1..7: note = Synthetic
TNVVKAG

SEQ ID NO: 538 - protein, 7 aa, synthetic construct
  REGION 1..7: note = Synthetic
FNQMKNV

SEQ ID NO: 539 - protein, 7 aa, synthetic construct
  REGION 1..7: note = Synthetic
MNSTKIW

SEQ ID NO: 540 - protein, 7 aa, synthetic construct
  REGION 1..7: note = Synthetic
TNYTKDP

SEQ ID NO: 541 - protein, 7 aa, synthetic construct
  REGION 1..7: note = Synthetic
FNSDKSR

SEQ ID NO: 542 - protein, 7 aa, synthetic construct
  REGION 1..7: note = Synthetic
NNAPKSS

SEQ ID NO: 543 - protein, 7 aa, synthetic construct
  REGION 1..7: note = Synthetic
TNYTKDP

SEQ ID NO: 544 - protein, 7 aa, synthetic construct
  REGION 1..7: note = Synthetic
FNTHREG

SEQ ID NO: 545 - protein, 7 aa, synthetic construct
  REGION 1..7: note = Synthetic
NNGMRSS

SEQ ID NO: 546 - protein, 7 aa, synthetic construct
  REGION 1..7: note = Synthetic
VNAIRSQ

SEQ ID NO: 547 - protein, 7 aa, synthetic construct
  REGION 1..7: note = Synthetic
FNVTRVQ

SEQ ID NO: 548 - protein, 7 aa, synthetic construct
  REGION 1..7: note = Synthetic
NNHMRHD

SEQ ID NO: 549 - protein, 7 aa, synthetic construct
  REGION 1..7: note = Synthetic
VNEVRNY

SEQ ID NO: 550 - protein, 7 aa, synthetic construct
  REGION 1..7: note = Synthetic
GNATKDQ

SEQ ID NO: 551 - protein, 7 aa, synthetic construct
  REGION 1..7: note = Synthetic
NNLTRSL

SEQ ID NO: 552 - protein, 7 aa, synthetic construct
  REGION 1..7: note = Synthetic
VNSNKHD

SEQ ID NO: 553 - protein, 7 aa, synthetic construct
  REGION 1..7: note = Synthetic
ANHIRSL

SEQ ID NO: 554 - protein, 7 aa, synthetic construct
  REGION 1..7: note = Synthetic
INHIRML

SEQ ID NO: 555 - protein, 7 aa, synthetic construct
  REGION 1..7: note = Synthetic
LNYTRSS

SEQ ID NO: 556 - protein, 7 aa, synthetic construct
  REGION 1..7: note = Synthetic
SNLRRTE

SEQ ID NO: 557 - protein, 7 aa, synthetic construct
  REGION 1..7: note = Synthetic
ANLARVT

SEQ ID NO: 558 - protein, 7 aa, synthetic construct
  REGION 1..7: note = Synthetic
INHLKSA

SEQ ID NO: 559 - protein, 7 aa, synthetic construct
  REGION 1..7: note = Synthetic
MNEVRYA

SEQ ID NO: 560 - protein, 7 aa, synthetic construct
  REGION 1..7: note = Synthetic
SNMPRNN

SEQ ID NO: 561 - protein, 7 aa, synthetic construct
  REGION 1..7: note = Synthetic
ANTGRHN

SEQ ID NO: 562 - protein, 7 aa, synthetic construct
  REGION 1..7: note = Synthetic
INHMRGA

SEQ ID NO: 563 - protein, 7 aa, synthetic construct
  REGION 1..7: note = Synthetic
MNHIRTT

SEQ ID NO: 564 - protein, 7 aa, synthetic construct
  REGION 1..7: note = Synthetic
SNNGRYM

SEQ ID NO: 565 - protein, 7 aa, synthetic construct
  REGION 1..7: note = Synthetic
ANYKRET

SEQ ID NO: 566 - protein, 7 aa, synthetic construct
  REGION 1..7: note = Synthetic
INMKRVP

SEQ ID NO: 567 - protein, 7 aa, synthetic construct
  REGION 1..7: note = Synthetic
MNNARPY

SEQ ID NO: 568 - protein, 7 aa, synthetic construct
  REGION 1..7: note = Synthetic
SNNVRHY

SEQ ID NO: 569 - protein, 7 aa, synthetic construct
  REGION 1..7: note = Synthetic
DNARRTL

SEQ ID NO: 570 - protein, 7 aa, synthetic construct
  REGION 1..7: note = Synthetic
INPRKDN

SEQ ID NO: 571 - protein, 7 aa, synthetic construct
  REGION 1..7: note = Synthetic
MNQIRAA

SEQ ID NO: 572 - protein, 7 aa, synthetic construct
  REGION 1..7: note = Synthetic
SNPTRQY

SEQ ID NO: 573 - protein, 7 aa, synthetic construct
  REGION 1..7: note = Synthetic
DNAVRPW

SEQ ID NO: 574 - protein, 7 aa, synthetic construct
  REGION 1..7: note = Synthetic
INQIRAA

SEQ ID NO: 575 - protein, 7 aa, synthetic construct
  REGION 1..7: note = Synthetic
MNQSRGW

SEQ ID NO: 576 - protein, 7 aa, synthetic construct
  REGION 1..7: note = Synthetic
SNRQREL

SEQ ID NO: 577 - protein, 7 aa, synthetic construct
  REGION 1..7: note = Synthetic
DNAVRSK

SEQ ID NO: 578 - protein, 7 aa, synthetic construct
  REGION 1..7: note = Synthetic
INQIRQV

SEQ ID NO: 579 - protein, 7 aa, synthetic construct
  REGION 1..7: note = Synthetic
MNRSRAE

SEQ ID NO: 580 - protein, 7 aa, synthetic construct
  REGION 1..7: note = Synthetic
SNSHKGW

SEQ ID NO: 581 - protein, 7 aa, synthetic construct
  REGION 1..7: note = Synthetic
DNEIRRW

SEQ ID NO: 582 - protein, 7 aa, synthetic construct
  REGION 1..7: note = Synthetic
INSIRMT

SEQ ID NO: 583 - protein, 7 aa, synthetic construct
  REGION 1..7: note = Synthetic
MNRTRFE

SEQ ID NO: 584 - protein, 7 aa, synthetic construct
  REGION 1..7: note = Synthetic
SNTGKSW

SEQ ID NO: 585 - protein, 7 aa, synthetic construct
  REGION 1..7: note = Synthetic
DNERRPR

SEQ ID NO: 586 - protein, 7 aa, synthetic construct
  REGION 1..7: note = Synthetic
INSLRLV

SEQ ID NO: 587 - protein, 7 aa, synthetic construct
  REGION 1..7: note = Synthetic
MNSPRSY

SEQ ID NO: 588 - protein, 7 aa, synthetic construct
  REGION 1..7: note = Synthetic
SNTIKNL

SEQ ID NO: 589 - protein, 7 aa, synthetic construct
  REGION 1..7: note = Synthetic
DNHIRSQ

SEQ ID NO: 590 - protein, 7 aa, synthetic construct
  REGION 1..7: note = Synthetic
INSPRLF

SEQ ID NO: 591 - protein, 7 aa, synthetic construct
  REGION 1..7: note = Synthetic
MNTIRSY

SEQ ID NO: 592 - protein, 7 aa, synthetic construct
  REGION 1..7: note = Synthetic
SNTVRTI

SEQ ID NO: 593 - protein, 7 aa, synthetic construct
  REGION 1..7: note = Synthetic
DNNPRKS

SEQ ID NO: 594 - protein, 7 aa, synthetic construct
  REGION 1..7: note = Synthetic
INSPRVT

SEQ ID NO: 595 - protein, 7 aa, synthetic construct
  REGION 1..7: note = Synthetic
MNTVRAW

SEQ ID NO: 596 - protein, 7 aa, synthetic construct
  REGION 1..7: note = Synthetic
TNDGRSR

SEQ ID NO: 597 - protein, 7 aa, synthetic construct
  REGION 1..7: note = Synthetic
DNPNKRQ

SEQ ID NO: 598 - protein, 7 aa, synthetic construct
  REGION 1..7: note = Synthetic
INSQRTP

SEQ ID NO: 599 - protein, 7 aa, synthetic construct
  REGION 1..7: note = Synthetic
MNTVRDY

SEQ ID NO: 600 - protein, 7 aa, synthetic construct
  REGION 1..7: note = Synthetic
TNFGRTT

SEQ ID NO: 601 - protein, 7 aa, synthetic construct
  REGION 1..7: note = Synthetic
DNVRRSN

SEQ ID NO: 602 - protein, 7 aa, synthetic construct
  REGION 1..7: note = Synthetic
INSTRYP

SEQ ID NO: 603 - protein, 7 aa, synthetic construct
  REGION 1..7: note = Synthetic
MNVLKKG

SEQ ID NO: 604 - protein, 7 aa, synthetic construct
  REGION 1..7: note = Synthetic
TNHIRFT

SEQ ID NO: 605 - protein, 7 aa, synthetic construct
  REGION 1..7: note = Synthetic
ENHLRNT

SEQ ID NO: 606 - protein, 7 aa, synthetic construct
  REGION 1..7: note = Synthetic
INSYKGA

SEQ ID NO: 607 - protein, 7 aa, synthetic construct
  REGION 1..7: note = Synthetic
NNALKPY

SEQ ID NO: 608 - protein, 7 aa, synthetic construct
  REGION 1..7: note = Synthetic
TNHIRNL

SEQ ID NO: 609 - protein, 7 aa, synthetic construct
  REGION 1..7: note = Synthetic
ENRSKNN

SEQ ID NO: 610 - protein, 7 aa, synthetic construct
  REGION 1..7: note = Synthetic
INTSRSA

SEQ ID NO: 611 - protein, 7 aa, synthetic construct
  REGION 1..7: note = Synthetic
NNFSRNG

SEQ ID NO: 612 - protein, 7 aa, synthetic construct
  REGION 1..7: note = Synthetic
TNMIRNA

SEQ ID NO: 613 - protein, 7 aa, synthetic construct
  REGION 1..7: note = Synthetic
ENSIRMY

SEQ ID NO: 614 - protein, 7 aa, synthetic construct
  REGION 1..7: note = Synthetic
INTVRDR

SEQ ID NO: 615 - protein, 7 aa, synthetic construct
  REGION 1..7: note = Synthetic
NNLNKYL

SEQ ID NO: 616 - protein, 7 aa, synthetic construct
  REGION 1..7: note = Synthetic
TNMPRNS

SEQ ID NO: 617 - protein, 7 aa, synthetic construct
  REGION 1..7: note = Synthetic
ENVVRSK

SEQ ID NO: 618 - protein, 7 aa, synthetic construct
  REGION 1..7: note = Synthetic
INTVRSS

SEQ ID NO: 619 - protein, 7 aa, synthetic construct
  REGION 1..7: note = Synthetic
NNMPRGG

SEQ ID NO: 620 - protein, 7 aa, synthetic construct
  REGION 1..7: note = Synthetic
TNPARYG

SEQ ID NO: 621 - protein, 7 aa, synthetic construct
  REGION 1..7: note = Synthetic
FNGGRMG

SEQ ID NO: 622 - protein, 7 aa, synthetic construct
  REGION 1..7: note = Synthetic
KNAIKLG

SEQ ID NO: 623 - protein, 7 aa, synthetic construct
  REGION 1..7: note = Synthetic
NNNVRFL

SEQ ID NO: 624 - protein, 7 aa, synthetic construct
  REGION 1..7: note = Synthetic
TNPNRSS

SEQ ID NO: 625 - protein, 7 aa, synthetic construct
  REGION 1..7: note = Synthetic
FNKTRGP

SEQ ID NO: 626 - protein, 7 aa, synthetic construct
  REGION 1..7: note = Synthetic
KNLLRSE

SEQ ID NO: 627 - protein, 7 aa, synthetic construct
  REGION 1..7: note = Synthetic
NNPLKRV

SEQ ID NO: 628 - protein, 7 aa, synthetic construct
  REGION 1..7: note = Synthetic
TNPSRFA

SEQ ID NO: 629 - protein, 7 aa, synthetic construct
  REGION 1..7: note = Synthetic
FNRERNN

SEQ ID NO: 630 - protein, 7 aa, synthetic construct
  REGION 1..7: note = Synthetic
LNERKYT

SEQ ID NO: 631 - protein, 7 aa, synthetic construct
  REGION 1..7: note = Synthetic
NNSARPL

SEQ ID NO: 632 - protein, 7 aa, synthetic construct
  REGION 1..7: note = Synthetic
TNQLRGY

SEQ ID NO: 633 - protein, 7 aa, synthetic construct
  REGION 1..7: note = Synthetic
FNTKRDF

SEQ ID NO: 634 - protein, 7 aa, synthetic construct
  REGION 1..7: note = Synthetic
LNERRYQ

SEQ ID NO: 635 - protein, 7 aa, synthetic construct
  REGION 1..7: note = Synthetic
NNSTRAG

SEQ ID NO: 636 - protein, 7 aa, synthetic construct
  REGION 1..7: note = Synthetic
TNQLRRD

SEQ ID NO: 637 - protein, 7 aa, synthetic construct
  REGION 1..7: note = Synthetic
FNTLKLG

SEQ ID NO: 638 - protein, 7 aa, synthetic construct
  REGION 1..7: note = Synthetic
LNGGRSW

SEQ ID NO: 639 - protein, 7 aa, synthetic construct
  REGION 1..7: note = Synthetic
NNSTRGG

SEQ ID NO: 640 - protein, 7 aa, synthetic construct
  REGION 1..7: note = Synthetic
TNSGKWS

SEQ ID NO: 641 - protein, 7 aa, synthetic construct
  REGION 1..7: note = Synthetic
FNTPRTA

SEQ ID NO: 642 - protein, 7 aa, synthetic construct
  REGION 1..7: note = Synthetic
LNHIRLS

SEQ ID NO: 643 - protein, 7 aa, synthetic construct
  REGION 1..7: note = Synthetic
NNTIRYS

SEQ ID NO: 644 - protein, 7 aa, synthetic construct
  REGION 1..7: note = Synthetic
TNSTRFT

SEQ ID NO: 645 - protein, 7 aa, synthetic construct
  REGION 1..7: note = Synthetic
GNARRET

SEQ ID NO: 646 - protein, 7 aa, synthetic construct
  REGION 1..7: note = Synthetic
LNHIRSL

SEQ ID NO: 647 - protein, 7 aa, synthetic construct
  REGION 1..7: note = Synthetic
NNVIRGF

SEQ ID NO: 648 - protein, 7 aa, synthetic construct
  REGION 1..7: note = Synthetic
VNDLRTR

SEQ ID NO: 649 - protein, 7 aa, synthetic construct
  REGION 1..7: note = Synthetic
GNAVKST

SEQ ID NO: 650 - protein, 7 aa, synthetic construct
  REGION 1..7: note = Synthetic
LNHMRNT

SEQ ID NO: 651 - protein, 7 aa, synthetic construct
  REGION 1..7: note = Synthetic
NNYPRNM

SEQ ID NO: 652 - protein, 7 aa, synthetic construct
  REGION 1..7: note = Synthetic
VNGHRSN

SEQ ID NO: 653 - protein, 7 aa, synthetic construct
  REGION 1..7: note = Synthetic
GNAWKNA

SEQ ID NO: 654 - protein, 7 aa, synthetic construct
  REGION 1..7: note = Synthetic
LNLNRYS

SEQ ID NO: 655 - protein, 7 aa, synthetic construct
  REGION 1..7: note = Synthetic
PNMPRYE

SEQ ID NO: 656 - protein, 7 aa, synthetic construct
  REGION 1..7: note = Synthetic
VNHVRLT

SEQ ID NO: 657 - protein, 7 aa, synthetic construct
  REGION 1..7: note = Synthetic
GNDIRVR

SEQ ID NO: 658 - protein, 7 aa, synthetic construct
  REGION 1..7: note = Synthetic
LNMSKYA

SEQ ID NO: 659 - protein, 7 aa, synthetic construct
  REGION 1..7: note = Synthetic
PNNLRNV

SEQ ID NO: 660 - protein, 7 aa, synthetic construct
  REGION 1..7: note = Synthetic
VNMLRDP

SEQ ID NO: 661 - protein, 7 aa, synthetic construct
  REGION 1..7: note = Synthetic
GNESRYM

SEQ ID NO: 662 - protein, 7 aa, synthetic construct
  REGION 1..7: note = Synthetic
LNNIRNV

SEQ ID NO: 663 - protein, 7 aa, synthetic construct
  REGION 1..7: note = Synthetic
PNNVRQY

SEQ ID NO: 664 - protein, 7 aa, synthetic construct
  REGION 1..7: note = Synthetic
VNMPRSV

SEQ ID NO: 665 - protein, 7 aa, synthetic construct
  REGION 1..7: note = Synthetic
GNEVRYY

SEQ ID NO: 666 - protein, 7 aa, synthetic construct
  REGION 1..7: note = Synthetic
LNNIRQV

SEQ ID NO: 667 - protein, 7 aa, synthetic construct
  REGION 1..7: note = Synthetic
PNRYKDV

SEQ ID NO: 668 - protein, 7 aa, synthetic construct
  REGION 1..7: note = Synthetic
VNNMRYP

SEQ ID NO: 669 - protein, 7 aa, synthetic construct
  REGION 1..7: note = Synthetic
GNGVKWI

SEQ ID NO: 670 - protein, 7 aa, synthetic construct
  REGION 1..7: note = Synthetic
LNNLRSP

SEQ ID NO: 671 - protein, 7 aa, synthetic construct
  REGION 1..7: note = Synthetic
PNSIRRD

SEQ ID NO: 672 - protein, 7 aa, synthetic construct
  REGION 1..7: note = Synthetic
VNSPRTP

SEQ ID NO: 673 - protein, 7 aa, synthetic construct
  REGION 1..7: note = Synthetic
GNKFKQA

SEQ ID NO: 674 - protein, 7 aa, synthetic construct
  REGION 1..7: note = Synthetic
LNNTKTA

SEQ ID NO: 675 - protein, 7 aa, synthetic construct
  REGION 1..7: note = Synthetic
PNSYKNI

SEQ ID NO: 676 - protein, 7 aa, synthetic construct
  REGION 1..7: note = Synthetic
VNTIRNV

SEQ ID NO: 677 - protein, 7 aa, synthetic construct
  REGION 1..7: note = Synthetic
GNLGRSS

SEQ ID NO: 678 - protein, 7 aa, synthetic construct
  REGION 1..7: note = Synthetic
LNNVRMI

SEQ ID NO: 679 - protein, 7 aa, synthetic construct
  REGION 1..7: note = Synthetic
PNTIRNV

SEQ ID NO: 680 - protein, 7 aa, synthetic construct
  REGION 1..7: note = Synthetic
WNAPRNA

SEQ ID NO: 681 - protein, 7 aa, synthetic construct
  REGION 1..7: note = Synthetic
GNMIRND

SEQ ID NO: 682 - protein, 7 aa, synthetic construct
  REGION 1..7: note = Synthetic
LNNVRSP

SEQ ID NO: 683 - protein, 7 aa, synthetic construct
  REGION 1..7: note = Synthetic
PNTLRYA

SEQ ID NO: 684 - protein, 7 aa, synthetic construct
  REGION 1..7: note = Synthetic
WNENKRL

SEQ ID NO: 685 - protein, 7 aa, synthetic construct
  REGION 1..7: note = Synthetic
GNMPRSN

SEQ ID NO: 686 - protein, 7 aa, synthetic construct
  REGION 1..7: note = Synthetic
LNNVRSV

SEQ ID NO: 687 - protein, 7 aa, synthetic construct
  REGION 1..7: note = Synthetic
QNNIKAW

SEQ ID NO: 688 - protein, 7 aa, synthetic construct
  REGION 1..7: note = Synthetic
WNSPRTN

SEQ ID NO: 689 - protein, 7 aa, synthetic construct
  REGION 1..7: note = Synthetic
GNNTKAV

SEQ ID NO: 690 - protein, 7 aa, synthetic construct
  REGION 1..7: note = Synthetic
LNPRKIG

SEQ ID NO: 691 - protein, 7 aa, synthetic construct
  REGION 1..7: note = Synthetic
QNNNRPA

SEQ ID NO: 692 - protein, 7 aa, synthetic construct
  REGION 1..7: note = Synthetic
YNNFKGN

SEQ ID NO: 693 - protein, 7 aa, synthetic construct
  REGION 1..7: note = Synthetic
GNNVKQF

SEQ ID NO: 694 - protein, 7 aa, synthetic construct
  REGION 1..7: note = Synthetic
LNQYRAA

SEQ ID NO: 695 - protein, 7 aa, synthetic construct
  REGION 1..7: note = Synthetic
QNRMRND

SEQ ID NO: 696 - protein, 7 aa, synthetic construct
  REGION 1..7: note = Synthetic
YNRNKFE

SEQ ID NO: 697 - protein, 7 aa, synthetic construct
  REGION 1..7: note = Synthetic
GNSARNI

SEQ ID NO: 698 - protein, 7 aa, synthetic construct
  REGION 1..7: note = Synthetic
LNRERSA

SEQ ID NO: 699 - protein, 7 aa, synthetic construct
  REGION 1..7: note = Synthetic
QNRQRDT

SEQ ID NO: 700 - protein, 7 aa, synthetic construct
  REGION 1..7: note = Synthetic
YNSGRNT

SEQ ID NO: 701 - protein, 7 aa, synthetic construct
  REGION 1..7: note = Synthetic
GNSIRAL

SEQ ID NO: 702 - protein, 7 aa, synthetic construct
  REGION 1..7: note = Synthetic
LNRVRDH

SEQ ID NO: 703 - protein, 7 aa, synthetic construct
  REGION 1..7: note = Synthetic
QNSLRYS

SEQ ID NO: 704 - protein, 7 aa, synthetic construct
  REGION 1..7: note = Synthetic
YNTGRLV

SEQ ID NO: 705 - protein, 7 aa, synthetic construct
  REGION 1..7: note = Synthetic
GNSIRDT

SEQ ID NO: 706 - protein, 7 aa, synthetic construct
  REGION 1..7: note = Synthetic
LNRVRGD

SEQ ID NO: 707 - protein, 7 aa, synthetic construct
  REGION 1..7: note = Synthetic
RNDTRHI

SEQ ID NO: 708 - protein, 7 aa, synthetic construct
  REGION 1..7: note = Synthetic
GNSIRTI

SEQ ID NO: 709 - protein, 7 aa, synthetic construct
  REGION 1..7: note = Synthetic
LNSARSI

SEQ ID NO: 710 - protein, 7 aa, synthetic construct
  REGION 1..7: note = Synthetic
RNFERAN

SEQ ID NO: 711 - protein, 7 aa, synthetic construct
  REGION 1..7: note = Synthetic
GNSSRNL

SEQ ID NO: 712 - protein, 7 aa, synthetic construct
  REGION 1..7: note = Synthetic
LNSGRSA

SEQ ID NO: 713 - protein, 7 aa, synthetic construct
  REGION 1..7: note = Synthetic
RNGFREL

SEQ ID NO: 714 - protein, 7 aa, synthetic construct
  REGION 1..7: note = Synthetic
GNSVRGW

SEQ ID NO: 715 - protein, 7 aa, synthetic construct
  REGION 1..7: note = Synthetic
LNSLRHP

SEQ ID NO: 716 - protein, 7 aa, synthetic construct
  REGION 1..7: note = Synthetic
RNPAKTG

SEQ ID NO: 717 - protein, 7 aa, synthetic construct
  REGION 1..7: note = Synthetic
GNTIRDI

SEQ ID NO: 718 - protein, 7 aa, synthetic construct
  REGION 1..7: note = Synthetic
LNSVRHV

SEQ ID NO: 719 - protein, 7 aa, synthetic construct
  REGION 1..7: note = Synthetic
RNQDRTT

SEQ ID NO: 720 - protein, 7 aa, synthetic construct
  REGION 1..7: note = Synthetic
GNVIRSH

SEQ ID NO: 721 - protein, 7 aa, synthetic construct
  REGION 1..7: note = Synthetic
LNTFKAA

SEQ ID NO: 722 - protein, 7 aa, synthetic construct
  REGION 1..7: note = Synthetic
RNVDRST

SEQ ID NO: 723 - protein, 7 aa, synthetic construct
  REGION 1..7: note = Synthetic
GNVPRVF

SEQ ID NO: 724 - protein, 7 aa, synthetic construct
  REGION 1..7: note = Synthetic
LNTIRST

SEQ ID NO: 725 - protein, 7 aa, synthetic construct
  REGION 1..7: note = Synthetic
RNYIKSD

SEQ ID NO: 726 - protein, 7 aa, synthetic construct
  REGION 1..7: note = Synthetic
GNVTRST

SEQ ID NO: 727 - protein, 7 aa, synthetic construct
  REGION 1..7: note = Synthetic
LNTLRTI

SEQ ID NO: 728 - protein, 7 aa, synthetic construct
  REGION 1..7: note = Synthetic
SNDGRYY

SEQ ID NO: 729 - protein, 7 aa, synthetic construct
  REGION 1..7: note = Synthetic
GNYGRAQ

SEQ ID NO: 730 - protein, 7 aa, synthetic construct
  REGION 1..7: note = Synthetic
LNTSRSW

SEQ ID NO: 731 - protein, 7 aa, synthetic construct
  REGION 1..7: note = Synthetic
SNHIRLA

SEQ ID NO: 732 - protein, 7 aa, synthetic construct
  REGION 1..7: note = Synthetic
GNYSRMD

SEQ ID NO: 733 - protein, 7 aa, synthetic construct
  REGION 1..7: note = Synthetic
LNTVRHI

SEQ ID NO: 734 - protein, 7 aa, synthetic construct
  REGION 1..7: note = Synthetic
SNHIRTL

SEQ ID NO: 735 - protein, 7 aa, synthetic construct
  REGION 1..7: note = Synthetic
HNDTKRY

SEQ ID NO: 736 - protein, 7 aa, synthetic construct
  REGION 1..7: note = Synthetic
LNYNKGY

SEQ ID NO: 737 - protein, 7 aa, synthetic construct
  REGION 1..7: note = Synthetic
SNHVRAI

SEQ ID NO: 738 - protein, 7 aa, synthetic construct
  REGION 1..7: note = Synthetic
INAPRTA

SEQ ID NO: 739 - protein, 7 aa, synthetic construct
  REGION 1..7: note = Synthetic
LNYTRPA

SEQ ID NO: 740 - protein, 7 aa, synthetic construct
  REGION 1..7: note = Synthetic
SNHVRFM

SEQ ID NO: 741 - protein, 7 aa, synthetic construct
  REGION 1..7: note = Synthetic
ANEVRRG

SEQ ID NO: 742 - protein, 7 aa, synthetic construct
  REGION 1..7: note = Synthetic
LNHIRSL

SEQ ID NO: 743 - protein, 7 aa, synthetic construct
  REGION 1..7: note = Synthetic
QNRMRND

SEQ ID NO: 744 - protein, 7 aa, synthetic construct
  REGION 1..7: note = Synthetic
ANHIRSL

SEQ ID NO: 745 - protein, 7 aa, synthetic construct
  REGION 1..7: note = Synthetic
LNHMRNT

SEQ ID NO: 746 - protein, 7 aa, synthetic construct
  REGION 1..7: note = Synthetic
QNTARFM

SEQ ID NO: 747 - protein, 7 aa, synthetic construct
  REGION 1..7: note = Synthetic
ANNNRNY

SEQ ID NO: 748 - protein, 7 aa, synthetic construct
  REGION 1..7: note = Synthetic
LNNIRNV

SEQ ID NO: 749 - protein, 7 aa, synthetic construct
  REGION 1..7: note = Synthetic
RNDTRHI

SEQ ID NO: 750 - protein, 7 aa, synthetic construct
  REGION 1..7: note = Synthetic
ANYKRET

SEQ ID NO: 751 - protein, 7 aa, synthetic construct
  REGION 1..7: note = Synthetic
LNNIRQV

SEQ ID NO: 752 - protein, 7 aa, synthetic construct
  REGION 1..7: note = Synthetic
RNYIKSD

SEQ ID NO: 753 - protein, 7 aa, synthetic construct
  REGION 1..7: note = Synthetic
CNGTKRE

SEQ ID NO: 754 - protein, 7 aa, synthetic construct
  REGION 1..7: note = Synthetic
LNNVRMI

SEQ ID NO: 755 - protein, 7 aa, synthetic construct
  REGION 1..7: note = Synthetic
SNHIRTL

SEQ ID NO: 756 - protein, 7 aa, synthetic construct
  REGION 1..7: note = Synthetic
CNRSRDG

SEQ ID NO: 757 - protein, 7 aa, synthetic construct
  REGION 1..7: note = Synthetic
LNNVRSV

SEQ ID NO: 758 - protein, 7 aa, synthetic construct
  REGION 1..7: note = Synthetic
SNHVRAI

SEQ ID NO: 759 - protein, 7 aa, synthetic construct
  REGION 1..7: note = Synthetic
DNARRTL

SEQ ID NO: 760 - protein, 7 aa, synthetic construct
  REGION 1..7: note = Synthetic
LNPRKIG

SEQ ID NO: 761 - protein, 7 aa, synthetic construct
  REGION 1..7: note = Synthetic
SNHVRTS

SEQ ID NO: 762 - protein, 7 aa, synthetic construct
  REGION 1..7: note = Synthetic
DNAVRSK

SEQ ID NO: 763 - protein, 7 aa, synthetic construct
  REGION 1..7: note = Synthetic
LNPVRNA

SEQ ID NO: 764 - protein, 7 aa, synthetic construct
  REGION 1..7: note = Synthetic
SNLRRTE

SEQ ID NO: 765 - protein, 7 aa, synthetic construct
  REGION 1..7: note = Synthetic
DNEIRRW

SEQ ID NO: 766 - protein, 7 aa, synthetic construct
  REGION 1..7: note = Synthetic
LNQYRAA

SEQ ID NO: 767 - protein, 7 aa, synthetic construct
  REGION 1..7: note = Synthetic
SNNVRHY

SEQ ID NO: 768 - protein, 7 aa, synthetic construct
  REGION 1..7: note = Synthetic
DNHIRSQ

SEQ ID NO: 769 - protein, 7 aa, synthetic construct
  REGION 1..7: note = Synthetic
LNRVRDH

SEQ ID NO: 770 - protein, 7 aa, synthetic construct
  REGION 1..7: note = Synthetic
SNPTRQY

SEQ ID NO: 771 - protein, 7 aa, synthetic construct
  REGION 1..7: note = Synthetic
ENHLRNT

SEQ ID NO: 772 - protein, 7 aa, synthetic construct
  REGION 1..7: note = Synthetic
LNRVRGD

SEQ ID NO: 773 - protein, 7 aa, synthetic construct
  REGION 1..7: note = Synthetic
SNRQREL

SEQ ID NO: 774 - protein, 7 aa, synthetic construct
  REGION 1..7: note = Synthetic
ENPPRPR

SEQ ID NO: 775 - protein, 7 aa, synthetic construct
  REGION 1..7: note = Synthetic
LNSARSI

SEQ ID NO: 776 - protein, 7 aa, synthetic construct
  REGION 1..7: note = Synthetic
SNSIRFI

SEQ ID NO: 777 - protein, 7 aa, synthetic construct
  REGION 1..7: note = Synthetic
ENRSKNN

SEQ ID NO: 778 - protein, 7 aa, synthetic construct
  REGION 1..7: note = Synthetic
LNSGRSA

SEQ ID NO: 779 - protein, 7 aa, synthetic construct
  REGION 1..7: note = Synthetic
SNTIKNL

SEQ ID NO: 780 - protein, 7 aa, synthetic construct
  REGION 1..7: note = Synthetic
ENSIRMY

SEQ ID NO: 781 - protein, 7 aa, synthetic construct
  REGION 1..7: note = Synthetic
LNSRREV

SEQ ID NO: 782 - protein, 7 aa, synthetic construct
  REGION 1..7: note = Synthetic
SNTIRTI

SEQ ID NO: 783 - protein, 7 aa, synthetic construct
  REGION 1..7: note = Synthetic
ENYGRGA

SEQ ID NO: 784 - protein, 7 aa, synthetic construct
  REGION 1..7: note = Synthetic
LNSVRHV

SEQ ID NO: 785 - protein, 7 aa, synthetic construct
  REGION 1..7: note = Synthetic
SNTVRTI

SEQ ID NO: 786 - protein, 7 aa, synthetic construct
  REGION 1..7: note = Synthetic
GNAVKST

SEQ ID NO: 787 - protein, 7 aa, synthetic construct
  REGION 1..7: note = Synthetic
LNTFKAA

SEQ ID NO: 788 - protein, 7 aa, synthetic construct
  REGION 1..7: note = Synthetic
TNFGRTT

SEQ ID NO: 789 - protein, 7 aa, synthetic construct
  REGION 1..7: note = Synthetic
GNDIRVR

SEQ ID NO: 790 - protein, 7 aa, synthetic construct
  REGION 1..7: note = Synthetic
LNTIRST

SEQ ID NO: 791 - protein, 7 aa, synthetic construct
  REGION 1..7: note = Synthetic
TNHIRFT

SEQ ID NO: 792 - protein, 7 aa, synthetic construct
  REGION 1..7: note = Synthetic
GNDIRYY

SEQ ID NO: 793 - protein, 7 aa, synthetic construct
  REGION 1..7: note = Synthetic
LNTIRTN

SEQ ID NO: 794 - protein, 7 aa, synthetic construct
  REGION 1..7: note = Synthetic
TNHIRNL

SEQ ID NO: 795 - protein, 7 aa, synthetic construct
  REGION 1..7: note = Synthetic
GNEVRYY

SEQ ID NO: 796 - protein, 7 aa, synthetic construct
  REGION 1..7: note = Synthetic
LNTIRVV

SEQ ID NO: 797 - protein, 7 aa, synthetic construct
  REGION 1..7: note = Synthetic
TNMIRNA

SEQ ID NO: 798 - protein, 7 aa, synthetic construct
  REGION 1..7: note = Synthetic
GNGVKWI

SEQ ID NO: 799 - protein, 7 aa, synthetic construct
  REGION 1..7: note = Synthetic
LNTVRDI

SEQ ID NO: 800 - protein, 7 aa, synthetic construct
  REGION 1..7: note = Synthetic
TNSTRFT

SEQ ID NO: 801 - protein, 7 aa, synthetic construct
  REGION 1..7: note = Synthetic
GNMIRND

SEQ ID NO: 802 - protein, 7 aa, synthetic construct
  REGION 1..7: note = Synthetic
LNTVRHI

SEQ ID NO: 803 - protein, 7 aa, synthetic construct
  REGION 1..7: note = Synthetic
TNVLRGF

SEQ ID NO: 804 - protein, 7 aa, synthetic construct
  REGION 1..7: note = Synthetic
GNNTKAV

SEQ ID NO: 805 - protein, 7 aa, synthetic construct
  REGION 1..7: note = Synthetic
LNYTRSS

SEQ ID NO: 806 - protein, 7 aa, synthetic construct
  REGION 1..7: note = Synthetic
VNHIRLQ

SEQ ID NO: 807 - protein, 7 aa, synthetic construct
  REGION 1..7: note = Synthetic
GNNVKQF

SEQ ID NO: 808 - protein, 7 aa, synthetic construct
  REGION 1..7: note = Synthetic
MNDVRSV

SEQ ID NO: 809 - protein, 7 aa, synthetic construct
  REGION 1..7: note = Synthetic
VNHVRLT

SEQ ID NO: 810 - protein, 7 aa, synthetic construct
  REGION 1..7: note = Synthetic
GNSARNI

SEQ ID NO: 811 - protein, 7 aa, synthetic construct
  REGION 1..7: note = Synthetic
MNHIRTT

SEQ ID NO: 812 - protein, 7 aa, synthetic construct
  REGION 1..7: note = Synthetic
VNNERSK

SEQ ID NO: 813 - protein, 7 aa, synthetic construct
  REGION 1..7: note = Synthetic
GNSIRAL

SEQ ID NO: 814 - protein, 7 aa, synthetic construct
  REGION 1..7: note = Synthetic
MNHPRMI

SEQ ID NO: 815 - protein, 7 aa, synthetic construct
  REGION 1..7: note = Synthetic
VNSPRSI

SEQ ID NO: 816 - protein, 7 aa, synthetic construct
  REGION 1..7: note = Synthetic
GNSIRDT

SEQ ID NO: 817 - protein, 7 aa, synthetic construct
  REGION 1..7: note = Synthetic
MNNARPY

SEQ ID NO: 818 - protein, 7 aa, synthetic construct
  REGION 1..7: note = Synthetic
VNTIRNV

SEQ ID NO: 819 - protein, 7 aa, synthetic construct
  REGION 1..7: note = Synthetic
GNSIRTI

SEQ ID NO: 820 - protein, 7 aa, synthetic construct
  REGION 1..7: note = Synthetic
MNQSRGW

SEQ ID NO: 821 - protein, 7 aa, synthetic construct
  REGION 1..7: note = Synthetic
VNTIRSV

SEQ ID NO: 822 - protein, 7 aa, synthetic construct
  REGION 1..7: note = Synthetic
GNSMRHM

SEQ ID NO: 823 - protein, 7 aa, synthetic construct
  REGION 1..7: note = Synthetic
MNRTRFE

SEQ ID NO: 824 - protein, 7 aa, synthetic construct
  REGION 1..7: note = Synthetic
WNSPRTN

SEQ ID NO: 825 - protein, 7 aa, synthetic construct
  REGION 1..7: note = Synthetic
GNSSRNL

SEQ ID NO: 826 - protein, 7 aa, synthetic construct
  REGION 1..7: note = Synthetic
MNSPRSL

SEQ ID NO: 827 - protein, 7 aa, synthetic construct
  REGION 1..7: note = Synthetic
YNRNKFE

SEQ ID NO: 828 - protein, 7 aa, synthetic construct
  REGION 1..7: note = Synthetic
GNSVRGW

SEQ ID NO: 829 - protein, 7 aa, synthetic construct
  REGION 1..7: note = Synthetic
MNTIRSY

SEQ ID NO: 830 - protein, 7 aa, synthetic construct
  REGION 1..7: note = Synthetic
YNSGRNT

SEQ ID NO: 831 - protein, 7 aa, synthetic construct
  REGION 1..7: note = Synthetic
GNTIRDI

SEQ ID NO: 832 - protein, 7 aa, synthetic construct
  REGION 1..7: note = Synthetic
MNTVRAW

SEQ ID NO: 833 - protein, 7 aa, synthetic construct
  REGION 1..7: note = Synthetic
YNTGRLV

SEQ ID NO: 834 - protein, 7 aa, synthetic construct
  REGION 1..7: note = Synthetic
GNVIRSH

SEQ ID NO: 835 - protein, 7 aa, synthetic construct
  REGION 1..7: note = Synthetic
MNTVRDY

SEQ ID NO: 836 - protein, 7 aa, synthetic construct
  REGION 1..7: note = Synthetic
GNVMRTT

SEQ ID NO: 837 - protein, 7 aa, synthetic construct
  REGION 1..7: note = Synthetic
NNASKGW

SEQ ID NO: 838 - protein, 7 aa, synthetic construct
  REGION 1..7: note = Synthetic
GNVTRST

SEQ ID NO: 839 - protein, 7 aa, synthetic construct
  REGION 1..7: note = Synthetic
NNFSRNG

SEQ ID NO: 840 - protein, 7 aa, synthetic construct
  REGION 1..7: note = Synthetic
GNYGRAQ

SEQ ID NO: 841 - protein, 7 aa, synthetic construct
  REGION 1..7: note = Synthetic
NNNVRFL

SEQ ID NO: 842 - protein, 7 aa, synthetic construct
  REGION 1..7: note = Synthetic
HNMSRIA

SEQ ID NO: 843 - protein, 7 aa, synthetic construct
  REGION 1..7: note = Synthetic
NNPVRIP

SEQ ID NO: 844 - protein, 7 aa, synthetic construct
  REGION 1..7: note = Synthetic
INHLKSA

SEQ ID NO: 845 - protein, 7 aa, synthetic construct
  REGION 1..7: note = Synthetic
NNSARPL

SEQ ID NO: 846 - protein, 7 aa, synthetic construct
  REGION 1..7: note = Synthetic
INHMRGA

SEQ ID NO: 847 - protein, 7 aa, synthetic construct
  REGION 1..7: note = Synthetic
NNSTRAG

SEQ ID NO: 848 - protein, 7 aa, synthetic construct
  REGION 1..7: note = Synthetic
INMPRDT

SEQ ID NO: 849 - protein, 7 aa, synthetic construct
  REGION 1..7: note = Synthetic
NNSTRGG

SEQ ID NO: 850 - protein, 7 aa, synthetic construct
  REGION 1..7: note = Synthetic
INNARIP

SEQ ID NO: 851 - protein, 7 aa, synthetic construct
  REGION 1..7: note = Synthetic
NNTIRYS

SEQ ID NO: 852 - protein, 7 aa, synthetic construct
  REGION 1..7: note = Synthetic
INQIRQV

SEQ ID NO: 853 - protein, 7 aa, synthetic construct
  REGION 1..7: note = Synthetic
NNVIRGF

SEQ ID NO: 854 - protein, 7 aa, synthetic construct
  REGION 1..7: note = Synthetic
INSPRLF

SEQ ID NO: 855 - protein, 7 aa, synthetic construct
  REGION 1..7: note = Synthetic
PNHVRIA

SEQ ID NO: 856 - protein, 7 aa, synthetic construct
  REGION 1..7: note = Synthetic
INSPRVS

SEQ ID NO: 857 - protein, 7 aa, synthetic construct
  REGION 1..7: note = Synthetic
PNNLRNV

SEQ ID NO: 858 - protein, 7 aa, synthetic construct
  REGION 1..7: note = Synthetic
INSPRVT

SEQ ID NO: 859 - protein, 7 aa, synthetic construct
  REGION 1..7: note = Synthetic
PNNVRQY

SEQ ID NO: 860 - protein, 7 aa, synthetic construct
  REGION 1..7: note = Synthetic
INTSRSA

SEQ ID NO: 861 - protein, 7 aa, synthetic construct
  REGION 1..7: note = Synthetic
PNSIRFS

SEQ ID NO: 862 - protein, 7 aa, synthetic construct
  REGION 1..7: note = Synthetic
INTVRDR

SEQ ID NO: 863 - protein, 7 aa, synthetic construct
  REGION 1..7: note = Synthetic
PNSIRRD

SEQ ID NO: 864 - protein, 7 aa, synthetic construct
  REGION 1..7: note = Synthetic
INTVRSS

SEQ ID NO: 865 - protein, 7 aa, synthetic construct
  REGION 1..7: note = Synthetic
PNTIRNV

SEQ ID NO: 866 - protein, 7 aa, synthetic construct
  REGION 1..7: note = Synthetic
KNAIKLG

SEQ ID NO: 867 - protein, 7 aa, synthetic construct
  REGION 1..7: note = Synthetic
PNTLRYA

SEQ ID NO: 868 - protein, 7 aa, synthetic construct
  REGION 1..7: note = Synthetic
KNNLREY

SEQ ID NO: 869 - protein, 7 aa, synthetic construct
  REGION 1..7: note = Synthetic
QNLGRYV

SEQ ID NO: 870 - protein, 7 aa, synthetic construct
  REGION 1..7: note = Synthetic
LNEVKLY

SEQ ID NO: 871 - protein, 7 aa, synthetic construct
  REGION 1..7: note = Synthetic
QNNIKAW

SEQ ID NO: 872 - protein, 7 aa, synthetic construct
  REGION 1..7: note = Synthetic
ANGTRGH

SEQ ID NO: 873 - protein, 7 aa, synthetic construct
  REGION 1..7: note = Synthetic
INHVRCV

SEQ ID NO: 874 - protein, 7 aa, synthetic construct
  REGION 1..7: note = Synthetic
LNYNKGY

SEQ ID NO: 875 - protein, 7 aa, synthetic construct
  REGION 1..7: note = Synthetic
SNHIRLA

SEQ ID NO: 876 - protein, 7 aa, synthetic construct
  REGION 1..7: note = Synthetic
ANHIRSL

SEQ ID NO: 877 - protein, 7 aa, synthetic construct
  REGION 1..7: note = Synthetic
INIIRVP

SEQ ID NO: 878 - protein, 7 aa, synthetic construct
  REGION 1..7: note = Synthetic
LNYTRPA

SEQ ID NO: 879 - protein, 7 aa, synthetic construct
  REGION 1..7: note = Synthetic
SNHIRTL

SEQ ID NO: 880 - protein, 7 aa, synthetic construct
  REGION 1..7: note = Synthetic
ANKNKPV

SEQ ID NO: 881 - protein, 7 aa, synthetic construct
  REGION 1..7: note = Synthetic
INITRNH

SEQ ID NO: 882 - protein, 7 aa, synthetic construct
  REGION 1..7: note = Synthetic
LNYTRSS

SEQ ID NO: 883 - protein, 7 aa, synthetic construct
  REGION 1..7: note = Synthetic
SNHRRME

SEQ ID NO: 884 - protein, 7 aa, synthetic construct
  REGION 1..7: note = Synthetic
ANRDRYT

SEQ ID NO: 885 - protein, 7 aa, synthetic construct
  REGION 1..7: note = Synthetic
INMKRVP

SEQ ID NO: 886 - protein, 7 aa, synthetic construct
  REGION 1..7: note = Synthetic
MNHIRTT

SEQ ID NO: 887 - protein, 7 aa, synthetic construct
  REGION 1..7: note = Synthetic
SNHVRAI

SEQ ID NO: 888 - protein, 7 aa, synthetic construct
  REGION 1..7: note = Synthetic
ANSQRHS

SEQ ID NO: 889 - protein, 7 aa, synthetic construct
  REGION 1..7: note = Synthetic
INPKRTA

SEQ ID NO: 890 - protein, 7 aa, synthetic construct
  REGION 1..7: note = Synthetic
MNHPRMI

SEQ ID NO: 891 - protein, 7 aa, synthetic construct
  REGION 1..7: note = Synthetic
SNHVRFM

SEQ ID NO: 892 - protein, 7 aa, synthetic construct
  REGION 1..7: note = Synthetic
ANYKRET

SEQ ID NO: 893 - protein, 7 aa, synthetic construct
  REGION 1..7: note = Synthetic
INQIRAA

SEQ ID NO: 894 - protein, 7 aa, synthetic construct
  REGION 1..7: note = Synthetic
MNHYRPQ

SEQ ID NO: 895 - protein, 7 aa, synthetic construct
  REGION 1..7: note = Synthetic
SNHVRTS

SEQ ID NO: 896 - protein, 7 aa, synthetic construct
  REGION 1..7: note = Synthetic
CNKPKDN

SEQ ID NO: 897 - protein, 7 aa, synthetic construct
  REGION 1..7: note = Synthetic
INSIRMT

SEQ ID NO: 898 - protein, 7 aa, synthetic construct
  REGION 1..7: note = Synthetic
MNLSKYP

SEQ ID NO: 899 - protein, 7 aa, synthetic construct
  REGION 1..7: note = Synthetic
SNKNRWE

SEQ ID NO: 900 - protein, 7 aa, synthetic construct
  REGION 1..7: note = Synthetic
DNARRVT

SEQ ID NO: 901 - protein, 7 aa, synthetic construct
  REGION 1..7: note = Synthetic
INSKKNA

SEQ ID NO: 902 - protein, 7 aa, synthetic construct
  REGION 1..7: note = Synthetic
MNMPRTS

SEQ ID NO: 903 - protein, 7 aa, synthetic construct
  REGION 1..7: note = Synthetic
SNLRRTE

SEQ ID NO: 904 - protein, 7 aa, synthetic construct
  REGION 1..7: note = Synthetic
DNAVRSK

SEQ ID NO: 905 - protein, 7 aa, synthetic construct
  REGION 1..7: note = Synthetic
INSPRVT

SEQ ID NO: 906 - protein, 7 aa, synthetic construct
  REGION 1..7: note = Synthetic
MNNARPY

SEQ ID NO: 907 - protein, 7 aa, synthetic construct
  REGION 1..7: note = Synthetic
SNNGRYM

SEQ ID NO: 908 - protein, 7 aa, synthetic construct
  REGION 1..7: note = Synthetic
DNEIRRW

SEQ ID NO: 909 - protein, 7 aa, synthetic construct
  REGION 1..7: note = Synthetic
INSTRYP

SEQ ID NO: 910 - protein, 7 aa, synthetic construct
  REGION 1..7: note = Synthetic
MNNSRMP

SEQ ID NO: 911 - protein, 7 aa, synthetic construct
  REGION 1..7: note = Synthetic
SNNVRHY

SEQ ID NO: 912 - protein, 7 aa, synthetic construct
  REGION 1..7: note = Synthetic
DNERRPR

SEQ ID NO: 913 - protein, 7 aa, synthetic construct
  REGION 1..7: note = Synthetic
INSVRIP

SEQ ID NO: 914 - protein, 7 aa, synthetic construct
  REGION 1..7: note = Synthetic
MNQSRGW

SEQ ID NO: 915 - protein, 7 aa, synthetic construct
  REGION 1..7: note = Synthetic
SNRDRNS

SEQ ID NO: 916 - protein, 7 aa, synthetic construct
  REGION 1..7: note = Synthetic
DNHIRSQ

SEQ ID NO: 917 - protein, 7 aa, synthetic construct
  REGION 1..7: note = Synthetic
INSYKGA

SEQ ID NO: 918 - protein, 7 aa, synthetic construct
  REGION 1..7: note = Synthetic
MNRSRAE

SEQ ID NO: 919 - protein, 7 aa, synthetic construct
  REGION 1..7: note = Synthetic
SNRNRDY

SEQ ID NO: 920 - protein, 7 aa, synthetic construct
  REGION 1..7: note = Synthetic
DNNPRKS

SEQ ID NO: 921 - protein, 7 aa, synthetic construct
  REGION 1..7: note = Synthetic
INTVRSS

SEQ ID NO: 922 - protein, 7 aa, synthetic construct
  REGION 1..7: note = Synthetic
MNRTRFE

SEQ ID NO: 923 - protein, 7 aa, synthetic construct
  REGION 1..7: note = Synthetic
SNSHKGW

SEQ ID NO: 924 - protein, 7 aa, synthetic construct
  REGION 1..7: note = Synthetic
DNVRRSN

SEQ ID NO: 925 - protein, 7 aa, synthetic construct
  REGION 1..7: note = Synthetic
INYQKPA

SEQ ID NO: 926 - protein, 7 aa, synthetic construct
  REGION 1..7: note = Synthetic
MNSPRSL

SEQ ID NO: 927 - protein, 7 aa, synthetic construct
  REGION 1..7: note = Synthetic
SNSIRFI

SEQ ID NO: 928 - protein, 7 aa, synthetic construct
  REGION 1..7: note = Synthetic
ENDKKNK

SEQ ID NO: 929 - protein, 7 aa, synthetic construct
  REGION 1..7: note = Synthetic
INYVKHH

SEQ ID NO: 930 - protein, 7 aa, synthetic construct
  REGION 1..7: note = Synthetic
MNSPRSY

SEQ ID NO: 931 - protein, 7 aa, synthetic construct
  REGION 1..7: note = Synthetic
SNTGKSW

SEQ ID NO: 932 - protein, 7 aa, synthetic construct
  REGION 1..7: note = Synthetic
ENMPRPT

SEQ ID NO: 933 - protein, 7 aa, synthetic construct
  REGION 1..7: note = Synthetic
KNAIKLG

SEQ ID NO: 934 - protein, 7 aa, synthetic construct
  REGION 1..7: note = Synthetic
MNTIRSY

SEQ ID NO: 935 - protein, 7 aa, synthetic construct
  REGION 1..7: note = Synthetic
SNTIKNL

SEQ ID NO: 936 - protein, 7 aa, synthetic construct
  REGION 1..7: note = Synthetic
ENNGRRN

SEQ ID NO: 937 - protein, 7 aa, synthetic construct
  REGION 1..7: note = Synthetic
KNALKSS

SEQ ID NO: 938 - protein, 7 aa, synthetic construct
  REGION 1..7: note = Synthetic
MNTVRAW

SEQ ID NO: 939 - protein, 7 aa, synthetic construct
  REGION 1..7: note = Synthetic
SNTIRTI

SEQ ID NO: 940 - protein, 7 aa, synthetic construct
  REGION 1..7: note = Synthetic
ENNRRQM

SEQ ID NO: 941 - protein, 7 aa, synthetic construct
  REGION 1..7: note = Synthetic
KNDERHR

SEQ ID NO: 942 - protein, 7 aa, synthetic construct
  REGION 1..7: note = Synthetic
MNVLKKG

SEQ ID NO: 943 - protein, 7 aa, synthetic construct
  REGION 1..7: note = Synthetic
TNERKYL

SEQ ID NO: 944 - protein, 7 aa, synthetic construct
  REGION 1..7: note = Synthetic
ENRQKYT

SEQ ID NO: 945 - protein, 7 aa, synthetic construct
  REGION 1..7: note = Synthetic
KNGGKPN

SEQ ID NO: 946 - protein, 7 aa, synthetic construct
  REGION 1..7: note = Synthetic
NNFSRNG

SEQ ID NO: 947 - protein, 7 aa, synthetic construct
  REGION 1..7: note = Synthetic
TNFGRTT

SEQ ID NO: 948 - protein, 7 aa, synthetic construct
  REGION 1..7: note = Synthetic
ENRSKNN

SEQ ID NO: 949 - protein, 7 aa, synthetic construct
  REGION 1..7: note = Synthetic
KNHNKPG

SEQ ID NO: 950 - protein, 7 aa, synthetic construct
  REGION 1..7: note = Synthetic
NNILRVG

SEQ ID NO: 951 - protein, 7 aa, synthetic construct
  REGION 1..7: note = Synthetic
TNHIRFT

SEQ ID NO: 952 - protein, 7 aa, synthetic construct
  REGION 1..7: note = Synthetic
ENSLRHR

SEQ ID NO: 953 - protein, 7 aa, synthetic construct
  REGION 1..7: note = Synthetic
KNLMRID

SEQ ID NO: 954 - protein, 7 aa, synthetic construct
  REGION 1..7: note = Synthetic
NNMPRGG

SEQ ID NO: 955 - protein, 7 aa, synthetic construct
  REGION 1..7: note = Synthetic
TNHIRHL

SEQ ID NO: 956 - protein, 7 aa, synthetic construct
  REGION 1..7: note = Synthetic
FNGGRMG

SEQ ID NO: 957 - protein, 7 aa, synthetic construct
  REGION 1..7: note = Synthetic
KNPIKSS

SEQ ID NO: 958 - protein, 7 aa, synthetic construct
  REGION 1..7: note = Synthetic
NNPLKRV

SEQ ID NO: 959 - protein, 7 aa, synthetic construct
  REGION 1..7: note = Synthetic
TNHIRHP

SEQ ID NO: 960 - protein, 7 aa, synthetic construct
  REGION 1..7: note = Synthetic
FNKTRGP

SEQ ID NO: 961 - protein, 7 aa, synthetic construct
  REGION 1..7: note = Synthetic
LNDSRRP

SEQ ID NO: 962 - protein, 7 aa, synthetic construct
  REGION 1..7: note = Synthetic
NNPVRIP

SEQ ID NO: 963 - protein, 7 aa, synthetic construct
  REGION 1..7: note = Synthetic
TNHIRNL

SEQ ID NO: 964 - protein, 7 aa, synthetic construct
  REGION 1..7: note = Synthetic
FNRERGP

SEQ ID NO: 965 - protein, 7 aa, synthetic construct
  REGION 1..7: note = Synthetic
LNERRYQ

SEQ ID NO: 966 - protein, 7 aa, synthetic construct
  REGION 1..7: note = Synthetic
NNSTRAG

SEQ ID NO: 967 - protein, 7 aa, synthetic construct
  REGION 1..7: note = Synthetic
TNKTRPA

SEQ ID NO: 968 - protein, 7 aa, synthetic construct
  REGION 1..7: note = Synthetic
FNRERNN

SEQ ID NO: 969 - protein, 7 aa, synthetic construct
  REGION 1..7: note = Synthetic
LNHARYP

SEQ ID NO: 970 - protein, 7 aa, synthetic construct
  REGION 1..7: note = Synthetic
NNSTRGG

SEQ ID NO: 971 - protein, 7 aa, synthetic construct
  REGION 1..7: note = Synthetic
TNMPRNS

SEQ ID NO: 972 - protein, 7 aa, synthetic construct
  REGION 1..7: note = Synthetic
FNTKRDF

SEQ ID NO: 973 - protein, 7 aa, synthetic construct
  REGION 1..7: note = Synthetic
LNHGRTA

SEQ ID NO: 974 - protein, 7 aa, synthetic construct
  REGION 1..7: note = Synthetic
NNTIRYS

SEQ ID NO: 975 - protein, 7 aa, synthetic construct
  REGION 1..7: note = Synthetic
TNPARYG

SEQ ID NO: 976 - protein, 7 aa, synthetic construct
  REGION 1..7: note = Synthetic
FNTPRTA

SEQ ID NO: 977 - protein, 7 aa, synthetic construct
  REGION 1..7: note = Synthetic
LNHIRLS

SEQ ID NO: 978 - protein, 7 aa, synthetic construct
  REGION 1..7: note = Synthetic
NNTYRSS

SEQ ID NO: 979 - protein, 7 aa, synthetic construct
  REGION 1..7: note = Synthetic
TNQLRRD

SEQ ID NO: 980 - protein, 7 aa, synthetic construct
  REGION 1..7: note = Synthetic
GNAVKST

SEQ ID NO: 981 - protein, 7 aa, synthetic construct
  REGION 1..7: note = Synthetic
LNHIRSL

SEQ ID NO: 982 - protein, 7 aa, synthetic construct
  REGION 1..7: note = Synthetic
NNVIRGF

SEQ ID NO: 983 - protein, 7 aa, synthetic construct
  REGION 1..7: note = Synthetic
TNSGKWS

SEQ ID NO: 984 - protein, 7 aa, synthetic construct
  REGION 1..7: note = Synthetic
GNAWKNA

SEQ ID NO: 985 - protein, 7 aa, synthetic construct
  REGION 1..7: note = Synthetic
LNHIRTS

SEQ ID NO: 986 - protein, 7 aa, synthetic construct
  REGION 1..7: note = Synthetic
PNEYKAR

SEQ ID NO: 987 - protein, 7 aa, synthetic construct
  REGION 1..7: note = Synthetic
TNSIRLP

SEQ ID NO: 988 - protein, 7 aa, synthetic construct
  REGION 1..7: note = Synthetic
GNDIRVR

SEQ ID NO: 989 - protein, 7 aa, synthetic construct
  REGION 1..7: note = Synthetic
LNHMRNT

SEQ ID NO: 990 - protein, 7 aa, synthetic construct
  REGION 1..7: note = Synthetic
PNHPRHL

SEQ ID NO: 991 - protein, 7 aa, synthetic construct
  REGION 1..7: note = Synthetic
TNSLRHP

SEQ ID NO: 992 - protein, 7 aa, synthetic construct
  REGION 1..7: note = Synthetic
GNKFKQA

SEQ ID NO: 993 - protein, 7 aa, synthetic construct
  REGION 1..7: note = Synthetic
LNIRRGE

SEQ ID NO: 994 - protein, 7 aa, synthetic construct
  REGION 1..7: note = Synthetic
PNMPRYE

SEQ ID NO: 995 - protein, 7 aa, synthetic construct
  REGION 1..7: note = Synthetic
TNSLRSI

SEQ ID NO: 996 - protein, 7 aa, synthetic construct
  REGION 1..7: note = Synthetic
GNLGRSS

SEQ ID NO: 997 - protein, 7 aa, synthetic construct
  REGION 1..7: note = Synthetic
LNKLRGP

SEQ ID NO: 998 - protein, 7 aa, synthetic construct
  REGION 1..7: note = Synthetic
PNNLRTP

SEQ ID NO: 999 - protein, 7 aa, synthetic construct
  REGION 1..7: note = Synthetic
TNSTRFT

SEQ ID NO: 1000 - protein, 7 aa, synthetic construct
  REGION 1..7: note = Synthetic
GNLQRYQ

SEQ ID NO: 1001 - protein, 7 aa, synthetic construct
  REGION 1..7: note = Synthetic
LNLNRYS

SEQ ID NO: 1002 - protein, 7 aa, synthetic construct
  REGION 1..7: note = Synthetic
PNNVRQY

SEQ ID NO: 1003 - protein, 7 aa, synthetic construct
  REGION 1..7: note = Synthetic
VNEVRMA

SEQ ID NO: 1004 - protein, 7 aa, synthetic construct
  REGION 1..7: note = Synthetic
GNMIRND

SEQ ID NO: 1005 - protein, 7 aa, synthetic construct
  REGION 1..7: note = Synthetic
LNMPRTN

SEQ ID NO: 1006 - protein, 7 aa, synthetic construct
  REGION 1..7: note = Synthetic
PNRYKDV

SEQ ID NO: 1007 - protein, 7 aa, synthetic construct
  REGION 1..7: note = Synthetic
VNHIRLQ

SEQ ID NO: 1008 - protein, 7 aa, synthetic construct
  REGION 1..7: note = Synthetic
GNNCKAT

SEQ ID NO: 1009 - protein, 7 aa, synthetic construct
  REGION 1..7: note = Synthetic
LNMSKYA

SEQ ID NO: 1010 - protein, 7 aa, synthetic construct
  REGION 1..7: note = Synthetic
PNSLRAI

SEQ ID NO: 1011 - protein, 7 aa, synthetic construct
  REGION 1..7: note = Synthetic
VNHVRLT

SEQ ID NO: 1012 - protein, 7 aa, synthetic construct
  REGION 1..7: note = Synthetic
GNNTKAV

SEQ ID NO: 1013 - protein, 7 aa, synthetic construct
  REGION 1..7: note = Synthetic
LNNIRNV

SEQ ID NO: 1014 - protein, 7 aa, synthetic construct
  REGION 1..7: note = Synthetic
PNSLRER

SEQ ID NO: 1015 - protein, 7 aa, synthetic construct
  REGION 1..7: note = Synthetic
VNLHRSG

SEQ ID NO: 1016 - protein, 7 aa, synthetic construct
  REGION 1..7: note = Synthetic
GNNVKQF

SEQ ID NO: 1017 - protein, 7 aa, synthetic construct
  REGION 1..7: note = Synthetic
LNNIRQV

SEQ ID NO: 1018 - protein, 7 aa, synthetic construct
  REGION 1..7: note = Synthetic
PNSMRQV

SEQ ID NO: 1019 - protein, 7 aa, synthetic construct
  REGION 1..7: note = Synthetic
VNNLRTL

SEQ ID NO: 1020 - protein, 7 aa, synthetic construct
  REGION 1..7: note = Synthetic
GNSARNI

SEQ ID NO: 1021 - protein, 7 aa, synthetic construct
  REGION 1..7: note = Synthetic
LNNVRSP

SEQ ID NO: 1022 - protein, 7 aa, synthetic construct
  REGION 1..7: note = Synthetic
PNTIRNV

SEQ ID NO: 1023 - protein, 7 aa, synthetic construct
  REGION 1..7: note = Synthetic
VNNMRYP

SEQ ID NO: 1024 - protein, 7 aa, synthetic construct
  REGION 1..7: note = Synthetic
GNSIRAL

SEQ ID NO: 1025 - protein, 7 aa, synthetic construct
  REGION 1..7: note = Synthetic
LNNVRSV

SEQ ID NO: 1026 - protein, 7 aa, synthetic construct
  REGION 1..7: note = Synthetic
PNTLRFA

SEQ ID NO: 1027 - protein, 7 aa, synthetic construct
  REGION 1..7: note = Synthetic
VNPNRSG

SEQ ID NO: 1028 - protein, 7 aa, synthetic construct
  REGION 1..7: note = Synthetic
GNSIRDT

SEQ ID NO: 1029 - protein, 7 aa, synthetic construct
  REGION 1..7: note = Synthetic
LNPRKIG

SEQ ID NO: 1030 - protein, 7 aa, synthetic construct
  REGION 1..7: note = Synthetic
QNKHREM

SEQ ID NO: 1031 - protein, 7 aa, synthetic construct
  REGION 1..7: note = Synthetic
VNSLRQY

SEQ ID NO: 1032 - protein, 7 aa, synthetic construct
  REGION 1..7: note = Synthetic
GNSIRTI

SEQ ID NO: 1033 - protein, 7 aa, synthetic construct
  REGION 1..7: note = Synthetic
LNPVRNA

SEQ ID NO: 1034 - protein, 7 aa, synthetic construct
  REGION 1..7: note = Synthetic
QNNIKAW

SEQ ID NO: 1035 - protein, 7 aa, synthetic construct
  REGION 1..7: note = Synthetic
VNTGKGW

SEQ ID NO: 1036 - protein, 7 aa, synthetic construct
  REGION 1..7: note = Synthetic
GNSLRAY

SEQ ID NO: 1037 - protein, 7 aa, synthetic construct
  REGION 1..7: note = Synthetic
LNQYRAA

SEQ ID NO: 1038 - protein, 7 aa, synthetic construct
  REGION 1..7: note = Synthetic
QNNLKYL

SEQ ID NO: 1039 - protein, 7 aa, synthetic construct
  REGION 1..7: note = Synthetic
VNTIRNV

SEQ ID NO: 1040 - protein, 7 aa, synthetic construct
  REGION 1..7: note = Synthetic
GNSSRNL

SEQ ID NO: 1041 - protein, 7 aa, synthetic construct
  REGION 1..7: note = Synthetic
LNRERSA

SEQ ID NO: 1042 - protein, 7 aa, synthetic construct
  REGION 1..7: note = Synthetic
QNRMRND

SEQ ID NO: 1043 - protein, 7 aa, synthetic construct
  REGION 1..7: note = Synthetic
VNTPRHS

SEQ ID NO: 1044 - protein, 7 aa, synthetic construct
  REGION 1..7: note = Synthetic
GNSVRGW

SEQ ID NO: 1045 - protein, 7 aa, synthetic construct
  REGION 1..7: note = Synthetic
LNRVRDH

SEQ ID NO: 1046 - protein, 7 aa, synthetic construct
  REGION 1..7: note = Synthetic
QNRQRDT

SEQ ID NO: 1047 - protein, 7 aa, synthetic construct
  REGION 1..7: note = Synthetic
WNAPRNA

SEQ ID NO: 1048 - protein, 7 aa, synthetic construct
  REGION 1..7: note = Synthetic
GNTIRDI

SEQ ID NO: 1049 - protein, 7 aa, synthetic construct
  REGION 1..7: note = Synthetic
LNRVRGD

SEQ ID NO: 1050 - protein, 7 aa, synthetic construct
  REGION 1..7: note = Synthetic
QNSLRYS

SEQ ID NO: 1051 - protein, 7 aa, synthetic construct
  REGION 1..7: note = Synthetic
WNEYRSS

SEQ ID NO: 1052 - protein, 7 aa, synthetic construct
  REGION 1..7: note = Synthetic
GNTYRDY

SEQ ID NO: 1053 - protein, 7 aa, synthetic construct
  REGION 1..7: note = Synthetic
LNSARSI

SEQ ID NO: 1054 - protein, 7 aa, synthetic construct
  REGION 1..7: note = Synthetic
QNTARFM

SEQ ID NO: 1055 - protein, 7 aa, synthetic construct
  REGION 1..7: note = Synthetic
WNHPRAA

SEQ ID NO: 1056 - protein, 7 aa, synthetic construct
  REGION 1..7: note = Synthetic
GNVIRSH

SEQ ID NO: 1057 - protein, 7 aa, synthetic construct
  REGION 1..7: note = Synthetic
LNSLRHP

SEQ ID NO: 1058 - protein, 7 aa, synthetic construct
  REGION 1..7: note = Synthetic
RNDGRVA

SEQ ID NO: 1059 - protein, 7 aa, synthetic construct
  REGION 1..7: note = Synthetic
WNNFRPS

SEQ ID NO: 1060 - protein, 7 aa, synthetic construct
  REGION 1..7: note = Synthetic
GNVPRVF

SEQ ID NO: 1061 - protein, 7 aa, synthetic construct
  REGION 1..7: note = Synthetic
LNSPRDR

SEQ ID NO: 1062 - protein, 7 aa, synthetic construct
  REGION 1..7: note = Synthetic
RNEVRFA

SEQ ID NO: 1063 - protein, 7 aa, synthetic construct
  REGION 1..7: note = Synthetic
WNPGRAG

SEQ ID NO: 1064 - protein, 7 aa, synthetic construct
  REGION 1..7: note = Synthetic
GNVTRST

SEQ ID NO: 1065 - protein, 7 aa, synthetic construct
  REGION 1..7: note = Synthetic
LNSPRFV

SEQ ID NO: 1066 - protein, 7 aa, synthetic construct
  REGION 1..7: note = Synthetic
RNEVRFG

SEQ ID NO: 1067 - protein, 7 aa, synthetic construct
  REGION 1..7: note = Synthetic
WNSNRFE

SEQ ID NO: 1068 - protein, 7 aa, synthetic construct
  REGION 1..7: note = Synthetic
GNYGRAQ

SEQ ID NO: 1069 - protein, 7 aa, synthetic construct
  REGION 1..7: note = Synthetic
LNSPRIT

SEQ ID NO: 1070 - protein, 7 aa, synthetic construct
  REGION 1..7: note = Synthetic
RNFNKND

SEQ ID NO: 1071 - protein, 7 aa, synthetic construct
  REGION 1..7: note = Synthetic
WNSPRNT

SEQ ID NO: 1072 - protein, 7 aa, synthetic construct
  REGION 1..7: note = Synthetic
HNDTKRY

SEQ ID NO: 1073 - protein, 7 aa, synthetic construct
  REGION 1..7: note = Synthetic
LNSRREV

SEQ ID NO: 1074 - protein, 7 aa, synthetic construct
  REGION 1..7: note = Synthetic
RNMAKAP

SEQ ID NO: 1075 - protein, 7 aa, synthetic construct
  REGION 1..7: note = Synthetic
WNSPRTN

SEQ ID NO: 1076 - protein, 7 aa, synthetic construct
  REGION 1..7: note = Synthetic
HNEERKR

SEQ ID NO: 1077 - protein, 7 aa, synthetic construct
  REGION 1..7: note = Synthetic
LNSVRHV

SEQ ID NO: 1078 - protein, 7 aa, synthetic construct
  REGION 1..7: note = Synthetic
RNNGRPQ

SEQ ID NO: 1079 - protein, 7 aa, synthetic construct
  REGION 1..7: note = Synthetic
YNAHRGA

SEQ ID NO: 1080 - protein, 7 aa, synthetic construct
  REGION 1..7: note = Synthetic
INAPRTA

SEQ ID NO: 1081 - protein, 7 aa, synthetic construct
  REGION 1..7: note = Synthetic
LNTFKAA

SEQ ID NO: 1082 - protein, 7 aa, synthetic construct
  REGION 1..7: note = Synthetic
RNNPKPL

SEQ ID NO: 1083 - protein, 7 aa, synthetic construct
  REGION 1..7: note = Synthetic
YNMPRGA

SEQ ID NO: 1084 - protein, 7 aa, synthetic construct
  REGION 1..7: note = Synthetic
INDLRTP

SEQ ID NO: 1085 - protein, 7 aa, synthetic construct
  REGION 1..7: note = Synthetic
LNTIRST

SEQ ID NO: 1086 - protein, 7 aa, synthetic construct
  REGION 1..7: note = Synthetic
RNPAKTG

SEQ ID NO: 1087 - protein, 7 aa, synthetic construct
  REGION 1..7: note = Synthetic
YNNFKGN

SEQ ID NO: 1088 - protein, 7 aa, synthetic construct
  REGION 1..7: note = Synthetic
INHIRML

SEQ ID NO: 1089 - protein, 7 aa, synthetic construct
  REGION 1..7: note = Synthetic
LNTPRST

SEQ ID NO: 1090 - protein, 7 aa, synthetic construct
  REGION 1..7: note = Synthetic
RNVDRST

SEQ ID NO: 1091 - protein, 7 aa, synthetic construct
  REGION 1..7: note = Synthetic
YNQMRNT

SEQ ID NO: 1092 - protein, 7 aa, synthetic construct
  REGION 1..7: note = Synthetic
INHLKSA

SEQ ID NO: 1093 - protein, 7 aa, synthetic construct
  REGION 1..7: note = Synthetic
LNTSRSW

SEQ ID NO: 1094 - protein, 7 aa, synthetic construct
  REGION 1..7: note = Synthetic
RNYIKSD

SEQ ID NO: 1095 - protein, 7 aa, synthetic construct
  REGION 1..7: note = Synthetic
YNRNKFE

SEQ ID NO: 1096 - protein, 7 aa, synthetic construct
  REGION 1..7: note = Synthetic
INHMRGA

SEQ ID NO: 1097 - protein, 7 aa, synthetic construct
  REGION 1..7: note = Synthetic
LNTVRHI

SEQ ID NO: 1098 - protein, 7 aa, synthetic construct
  REGION 1..7: note = Synthetic
SNFIRSA

SEQ ID NO: 1099 - protein, 7 aa, synthetic construct
  REGION 1..7: note = Synthetic
YNTGRLV

SEQ ID NO: 1100 - protein, 7 aa, synthetic construct
  REGION 1..7: note = Synthetic
TALKPFL

SEQ ID NO: 1101 - protein, 7 aa, synthetic construct
  REGION 1..7: note = Synthetic
TTLKPFL

SEQ ID NO: 1102 - protein, 9 aa, synthetic construct
  REGION 1..9: note = Synthetic
DGTLAVPFK

SEQ ID NO: 1103 - protein, 7 aa, synthetic construct
  REGION 1..7: note = Synthetic
TLAVPFK

SEQ ID NO: 1104 - protein, 7 aa, synthetic construct
  REGION 1..7: note = Synthetic
NRGTEWD

SEQ ID NO: 1105 - protein, 7 aa, synthetic construct
  REGION 1..7: note = Synthetic
DSPAHPS

SEQ ID NO: 1106 - DNA, 21 bp, synthetic construct
  misc_feature 1..21: note = Synthetic
  source 1..21: mol_type = other DNA
ggaacccctagtgatggagtt

SEQ ID NO: 1107 - DNA, 16 bp, synthetic construct
  misc_feature 1..16: note = Synthetic
  source 1..16: mol_type = other DNA
cggcctcagtgagcga

SEQ ID NO: 1108 - DNA, 21 bp, synthetic construct
  source 1..21: mol_type = other DNA
  misc_feature 9^10: note = ZEN internal quencher
  misc_feature 1..21: note = Synthetic
  misc_feature >21: note = IABkFQ Iowa Black Fluorescent Quencher
  misc_feature <1: note = HEX dye
cactccctctctgcgcgctcg

SEQ ID NO: 1109 - DNA, 32 bp, synthetic construct
  misc_feature 1..32: note = Synthetic
  source 1..32: mol_type = other DNA
tgatgtcttatagagataagcattagggtctt

SEQ ID NO: 1110 - DNA, 27 bp, synthetic construct
  source 1..27: mol_type = other DNA
  misc_feature 1..27: note = Synthetic
taatctcataaaaaagcactcattgtg

SEQ ID NO: 1111 - DNA, 17 bp, synthetic construct
  source 1..17: mol_type = other DNA
  misc_feature 1..17: note = Synthetic
cccatcaaagcaatcac

(19) United States
(12) Patent Application Publication
(10) Pub. No.: US 2025/0352670 A1
(43) Pub. Date: Nov. 20, 2025

(54) DELIVERING GENES TO THE BRAIN ENDOTHELIUM TO TREAT LYSOSOMAL STORAGE DISORDER-DERIVED NEUROPATHOLOGY

(71) Applicant: The Broad Institute, Inc., Cambridge, MA (US)

(72) Inventors: Benjamin Deverman, Cambridge, MA (US); Jason Wu, Cambridge, MA (US); Nuria R. Botticello-Romero, Cambridge, MA (US); Thomas Beddow, Cambridge, MA (US)

(21) Appl. No.: 19/288,927

(22) Filed: Aug. 1, 2025

Related U.S. Application Data
(63) Continuation of application No. PCT/US2024/014157, filed on Feb. 2, 2024.
(60) Provisional application No. 63/483,195, filed on Feb. 3, 2023.

(51) Int. Cl. A61K 48/00; A61K 38/46; A61P 3/00; C07K 14/005; C12N 15/113; C12N 15/86

(52) U.S. Cl. CPC A61K 48/005; A61K 38/465; A61P 3/00; C07K 14/005; C12N 15/113; C12N 15/86; C12Y 301/06013; C12N 2310/141; C12N 2750/14122; C12N 2750/14143

(57) ABSTRACT

Applicants sought to express human iduronate-2-sulfatase (hIDS) in the brain endothelium of a mouse model of Mucopolysaccharidosis type II (MPSII, Hunter's syndrome) to enable enzyme secretion into the brain parenchyma. In this disorder, IDS deficiency results in the pathophysiological accumulation of heparan and dermatan sulfate GAGs. To test the hypothesis, Applicants chose AAV-BI30, an AAV9-derived capsid that has an enhanced in vivo tropism specific to the endothelium in the rodent CNS and can transduce human brain vascular endothelial cells in vitro more efficiently than AAV9. Applicants show that systemic delivery of AAV-BI30: hIDS restored IDS enzyme activity in the brain, liver, and serum of IDS-KO mice (FIG. 1). Importantly, AAV-BI30-mediated gene transfer resulted in the correction of GAG accumulation in the brain (FIG. 2). This effect was not observed when using the AAV-BI30 vector packaging a non-secreting version of hIDS. These findings highlight that targeting endothelial cells throughout the CNS is a promising approach for delivering enzymes across the BBB and restoring lysosomal metabolism.

### CROSS-REFERENCE TO RELATED APPLICATIONS

This application is a continuation application of PCT/US2024/014157, filed Feb. 2, 2024, which claims the benefit of U.S. Provisional Application No. 63/483,195, filed on Feb. 3, 2023. The entire contents of the above-identified applications are hereby fully incorporated herein by reference.

### STATEMENT REGARDING FEDERALLY SPONSORED RESEARCH

This invention was made with government support under Grant No. NS111689 awarded by the National Institutes of Health. The government has certain rights in the invention.

### SEQUENCE LISTING

The contents of the electronic sequence listing (BROD-5805US_ST26.xml, size is 1,333,443 bytes, and it was created on Aug. 1, 2025) is herein incorporated by reference in its entirety.

### TECHNICAL FIELD

The subject matter disclosed herein is generally directed to methods of treating lysosomal storage diseases using non-naturally occurring or engineered adeno-associated virus (AAV) vectors with improved transduction properties. Further, the non-naturally occurring or engineered AAV vectors comprise cargo capable of treating the lysosomal storage diseases and are designed to target, i.e., be delivered, to the central nervous system and, specifically, the endothelial cells of the central nervous system vasculature.

### BACKGROUND

Lysosomal storage disorders (LSDs) are a heterogeneous group of genetic diseases characterized by the absence of functional lysosomal enzymes or transporters. The ensuing progressive accumulation of lipids, glycosaminoglycans (GAGs), or glycoproteins results in damage to cells and tissues. The most devastating forms of LSDs involve damage to the central nervous system (CNS), which leads to cognitive impairments and premature death.

LSDs are a group of more than 70 rare, genetic diseases that cause deficiency of lysosomal enzymes and other proteins necessary for metabolism of biomolecules by lysosomes. This causes an accumulation of excess substrates, leading to organ dysfunction throughout the body and contributing to morbidity and mortality. At least 75% of LSDs affect the central nervous system (CNS), though existing treatments for LSDs still do not sufficiently target disease phenotypes in the CNS, nor do they provide a long-term cure (Sands, 2008). Standard of care for LSDs is enzyme replacement therapy (ERT), while other treatments include bone marrow transplantation, substrate reduction therapy, use of molecular chaperones for stabilization of enzymes, and gene therapy (Sheth, 2020). ERT, however, has disadvantages because it requires repeated dosages throughout a patient's lifetime, which negatively affects their quality of life and can be prohibitively expensive. Additionally, repeated infusion of enzymes may in some cases lead to immune response, and most recombinant enzymes cannot cross the blood brain barrier (BBB) to treat defects in the CNS (Brooks, 2003; Vogler, 2005).

Enzyme-replacement therapy is the current standard of care for many LSDs, but the inability of recombinant enzymes to cross the blood-brain barrier (BBB) remains a significant challenge. Adeno-associated viruses (AAVs) and lentiviral vectors (LVs) have gained popularity in clinical trials as gene therapy vectors for treating LSDs (Nagree, 2019; Sevin, 2021). AAVs, already used in several FDA- and EMA-approved gene therapies, have advantages to treating LSDs with neurological involvement due to the ability of certain serotypes to transduce neural cells either by direct intracerebral injection, delivery into the cerebrospinal fluid or to a lesser extent, BBB crossing after intravenous injection (Li, 2020). However, several studies have noted neurotoxicity after direct transduction of neural cells and may not be optimal in disease states where neural cells are already compromised (Golebioweski, 2017; Johnston, 2021). Additionally, direct intracerebral injection increases the risk of infection and procedural complications. Cross-correction, the concept in which transduced cells can secrete lysosomal enzymes to be endocytosed by non-transduced cells, is a mechanism that can address issues around delivering enzymes across the BBB without directly transducing neuronal cells.

### SUMMARY

In one aspect, the present disclosure provides a method of treating a subject at risk for, or suffering from, a lysosomal storage disease (LSD) comprising; administering to a subject in need thereof, a therapeutically effective amount a recombinant adeno-associated virus (rAAV) capsid comprising a vector, the vector comprising a transgene encoding a polypeptide effective to treat the LSD, wherein the rAAV comprises a modified capsid comprising a n-mer motif that increases transduction of endothelial cells of the CNS vasculature, the n-mer motif comprising or consisting of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2.

In example embodiments, the LSD is selected from the group consisting of Mucopolysaccharidosis (MPS), Sphingolipidosis, Oligosaccharidosis, Neuronal ceroid lipofuscinosis, Sialic acid disorders, Mucolipidosis, Lysosomal Acid lipase deficiency infantile and childhood/adult types, Pompe disease, Danon disease, Wolman's disease. and Cystinosis. In example embodiments, the LSD is a MPS. In example embodiments, the MPS is selected from the group consisting of Hurler syndrome (MPS I), Hunter syndrome (MPS II), San Filippo syndrome A (MPS IIIA), San Filippo B (MPS IIIB), San Filippo C (MPS IIIC), San Filippo D (MPS IIID), Morquio syndrome A (MPS IVA), Morquio syndrome B (MPS IVB), Scheie syndrome (MPS V), Maroteaux-Lamy syndrome (MPS VI), Sly syndrome (MPS VII), hyaluronidase deficiency (MPS IX), and Hurler-Scheie syndrome.

In example embodiments, the MPS is: Hurler Syndrome and the transgene encodes IDUA; Hunter Syndrome and the transgene encodes iduronate 2-sulfatase (IDS); San Filippo Syndrome A and the transgene encodes SGSH; San Filippo Syndrome B and the transgene encodes NAGLU; San Filippo Syndrome C and the transgene encodes HGSNAT; San Filippo Syndrome D and the transgene encodes GNS; Morquio Syndrome A and the transgene encodes GALNS; Morquio Syndrome B and the transgene encodes GLB1; Scheie Syndrome and the transgene encodes IDUA; Maroteaux-Lamy Syndrome and the transgene encodes Aryl sulfatase B; Sly Syndrome and the transgene encodes GUSB; Hurler-Scheie Syndrome and the transgene encodes IDUA; or any combination thereof.

In example embodiments, the one or more repeat elements are the hepatocyte-selective miR-122 repeat element. In example embodiments, X1, X3, X4, X6, and X7 is independently selected from the following groups; X1 is selected from the group consisting of G, M, T, S, N, D, L, H, P, I, V, Q, Y, W, F, A, E; X3 is selected from N, S, T, H, D, A, Y, M, Q, E, R, G, V; X4 is selected from T, V, I, A, M, S, H, W, N; X6 is selected from N, S, G, D, P, T, H, Q, A, Y; and X7 is selected from T, Y, W, N, V, I, H, M, S, G, A, Q, F, D, P, R, L. In example embodiments, The method of claim 1, wherein X1, X3, X4, X6, and X7 are independently selected from the following groups; X1 is selected from the group consisting of G, M, T, S, N, D; X3 is selected from the group consisting of N, S, T, H, D; X4 is selected from the group consisting of T, V, I, A; X6 is selected from the group consisting of N, S, G, D, P; and X7 is selected from T, Y, W, N, V, I, H, M, S, G, A, Q, F, D, P, R, L.

In example embodiments, X1 is R or K and X3, X4, X6 and X7 are D or E. In example embodiments, X1 is not R, K, or C; X3 is not W, F, K, C, I, P or, L; X4 is not Y, G, P, D, C, Q, R, K, E, F, L, or R; X6 is not R, I, W, V, F, C, L, E, or K; or X7 is not C, K, E. In example embodiments, the n-mer motif is selected from one of Table 1 to Table 6. In example embodiments, the n-mer motif is NNSTRGG (SEQ ID NO: 1), GNSARNI (SEQ ID NO: 2), GNSVRDF (SEQ ID NO: 3), or a combination thereof.

In example embodiments, the targeting moiety is part of a viral capsid protein. In example embodiments, the targeting motif is located between two amino acids of the viral capsid protein such that the n-mer is external to a viral capsid. In example embodiments, the viral capsid protein is an AAV viral capsid protein. In example embodiments, the targeting motif is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide. In example embodiments, the engineered AAV capsid protein comprises one or more mutations. In example embodiments, the one or more mutations comprise K449R of AAV9, or in an analogous position in an AAV1, AAV2, AAV3, AAV4, AAV5, AAV6, AAV7, AAV8, AAV rh.74, AAV rh.10 capsid polypeptide.

In one aspect, the present disclosure provides a rAAV comprising a vector, the vector comprising a transgene encoding IDS, wherein the rAAV comprises a modified capsid comprising a n-mer motif that increases transduction of endothelial cells of the CNS vasculature, the n-mer motif comprising or consisting of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2.

In example embodiments, the one or more repeat elements are the hepatocyte-selective miR-122 repeat element. In example embodiments, X1, X3, X4, X6, and X7 is independently selected from the following groups; X1 is selected from the group consisting of G, M, T, S, N, D, L, H, P, I, V, Q, Y, W, F, A, E; X3 is selected from N, S, T, H, D, A, Y, M, Q, E, R, G, V; X4 is selected from T, V, I, A, M, S, H, W, N; X6 is selected from N, S, G, D, P, T, H, Q, A, Y; and X7 is selected from T, Y, W, N, V, I, H, M, S, G, A, Q, F, D, P, R, L.

In example embodiments, X1, X3, X4, X6, and X7 are independently selected from the following groups; X1 is selected from the group consisting of G, M, T, S, N, D; X3 is selected from the group consisting of N, S, T, H, D; X4 is selected from the group consisting of T, V, I, A; X6 is selected from the group consisting of N, S, G, D, P; and X7 is selected from T, Y, W, N, V, I, H, M, S, G, A, Q, F, D, P, R, L. In example embodiments, X1 is R or K and X3, X4, X6 and X7 are D or E. In example embodiments, X1 is not R, K, or C; X3 is not W, F, K, C, I, P or, L; X4 is not Y, G, P, D, C, Q, R, K, E, F, L, or R; X6 is not R, I, W, V, F, C, L, E, or K; or X7 is not C, K, E.

In example embodiments, the n-mer motif is selected from one of Table 1 to Table 6. In example embodiments, the n-mer motif is NNSTRGG (SEQ ID NO: 1), GNSARNI (SEQ ID NO: 2), GNSVRDF (SEQ ID NO: 3), or a combination thereof.

In example embodiments, the targeting moiety is part of a viral capsid protein. In example embodiments, the targeting motif is located between two amino acids of the viral capsid protein such that the n-mer is external to a viral capsid. In example embodiments, the viral capsid protein is an AAV viral capsid protein. In example embodiments, the targeting motif is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide. In example embodiments, the engineered AAV capsid protein comprises one or more mutations. In example embodiments, the one or more mutations comprise K449R of AAV9, or in an analogous position in an AAV1, AAV2, AAV3, AAV4, AAV5, AAV6, AAV7, AAV8, AAV rh.74, AAV rh.10 capsid polypeptide.

In one aspect, the present disclosure provides a pharmaceutical composition comprising the rAAV as described herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1: Vector Design—The human IDS gene (hIDS) with a C-terminal myc tag was synthesized and cloned into a pAAV expression vector. The complete ITR-flanked sequence included a CAG synthetic promoter driving hIDS-myc, followed by a woodchuck hepatitis virus post-transcriptional regulatory element (WPRE) and a human growth hormone (hGH) polyadenylation signal.

FIG. 2A-2C: AAV-BI30 enables in-vitro delivery, secretion, and uptake of hIDS-myc. (2A) HEK293T cells were transduced using AAV-BI30:hIDS-myc. Transgene expression was assessed by immunostaining of fixed cells using an anti-IDS antibody. Representative images demonstrate dose-dependent transduction in comparison to IDS expressed by transient transfection. (2B) Cell lysate and conditioned media were collected from HEK293T cells transiently transfected with hIDS. Enzyme concentration was determined using ELISA. A considerable portion of the overexpressed IDS is secreted into the media. (2C) HT1080 cells were treated with AAV-BI30:hIDS at an MOI of 1×10⁵. After 24 hours the virus was replaced with fresh media. After 3 days, the pre-conditioned media was transferred to untreated cells. The uptake of soluble enzyme was confirmed by immunostaining.

FIG. 3A-3C: Systemic administration of AAV-BI30 leads to reliable expression of hIDS by the brain vasculature-Adult C57BL/6J mice received intra-venous administration of 1×10¹¹ vg/animal AAV-BI30:hIDS. Blood serum and tissues were collected 3 weeks post-injection. (3A-B) Representative images of immunostaining of brain and liver sections using an anti-hIDS antibody. Consistent with known AAV-BI30 tropism in the CNS, hIDS is expressed primarily in endothelial cells and rare neurons (right). (3C) Concentration of hIDS in the mouse serum was measured by ELISA. AAV-mediated gene transfer led to substantial secretion of the enzyme into the bloodstream.

FIG. 4A-4B: Somatic IDS enzyme activity is restored in MPSII mice following AAV-mediated gene transfer—(4A-4B) Measurement of iduronate-2-sulfatase (IDS) activity in serum (4A) and liver (4B) samples obtained from WT and IDS-deficient mice treated with 3×1011 vg/mouse of AAV vectors encoding hIDS-myc or hIDS-NS-myc (no signal sequence) at 4-7 weeks of age. Tissues were collected 4 weeks after treatment. Enzyme activity was estimated by determining the fluorescence of the product of a sequential reaction partially catalyzed by IDS. Results are normalized by protein concentration. AAV-mediated gene transfer resulted in supraphysiological enzyme activity in the blood and liver of treated mice. Without enzyme secretion this effect is substantially reduced. Results are shown as mean+SD of 3-5 animals per group.

FIG. 5A-5C: Treatment with AAV-BI30:hIDS corrects pathological GAG accumulation in IDS-deficient mice—(5A-5C) Quantification of glycosaminoglycan (GAG) content in the brain (5A), liver (5B), and urine (5C) of 8- to 11-week-old WT and IDS-deficient mice 4 weeks after i.v. treatment with 3×10¹¹ vg/animal of therapeutic vectors with distinct CNS tropisms. In contrast with the naturally occurring AAV9, AAV-PHP.eB is known to broadly and efficiently target the cells of the CNS. Comparable correction of GAG accumulation observed in mice treated with AAV-BI30:hIDS suggests endothelial cell transduction can result in cross-correction of enzyme deficiency in neurons and glial cells. Results are shown as mean±SD of 3-5 animals per group.

FIG. 6: Systemic administration of AAV-BI30:hIDS corrects pathological glycosaminoglycan accumulation in IDS-deficient mice—Glycosaminoglycan (GAG) content in the brains of 8- to 11-week-old wild type (WT) and IDS-knockout (KO) mice was quantified four weeks after intravenous treatment with 3×1011 vg/animal of therapeutic vectors with distinct CNS tropisms. In comparison with the naturally occurring AAV9, AAV-PHP.eB is known to broadly and efficiently target the cells of the mouse CNS after systemic administration. Comparable correction of GAG accumulation in mice treated with AAV-BI30:hIDS or PHP.eB:hIDS suggests that gene delivery to the CNS endothelium can result in cross-correction of enzyme deficiency in neurons and glial cells to the same extent as gene delivery to the brain parenchyma. The results are normalized to the IDS-WT control. Lines represent the median.

FIG. 7A-7E: AAV-mediated gene therapy does not affect locomotor activity or exploration in young, IDS-deficient mice—(7A-7E) IDS-KO and WT mice were evaluated using the open-field test 4 weeks after AAV-mediated gene delivery. The mice were 8-11 weeks old and behavioral abnormalities related to MPSII pathology were not yet expected. There were no notable differences observed in locomotor or exploratory activity measurements (7A-7D) between treated and untreated mice, regardless of their IDS genotype. Similarly, no heighted anxiety (7E) was observed in any of the groups.

FIG. 8: in vitro characterization of transfected HEK293T using ELISA against IDS in the cell media and cell lysate.

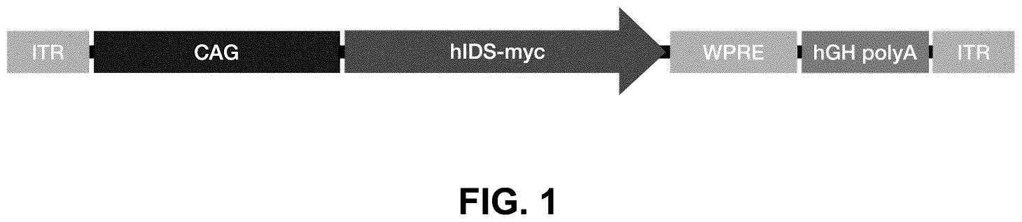

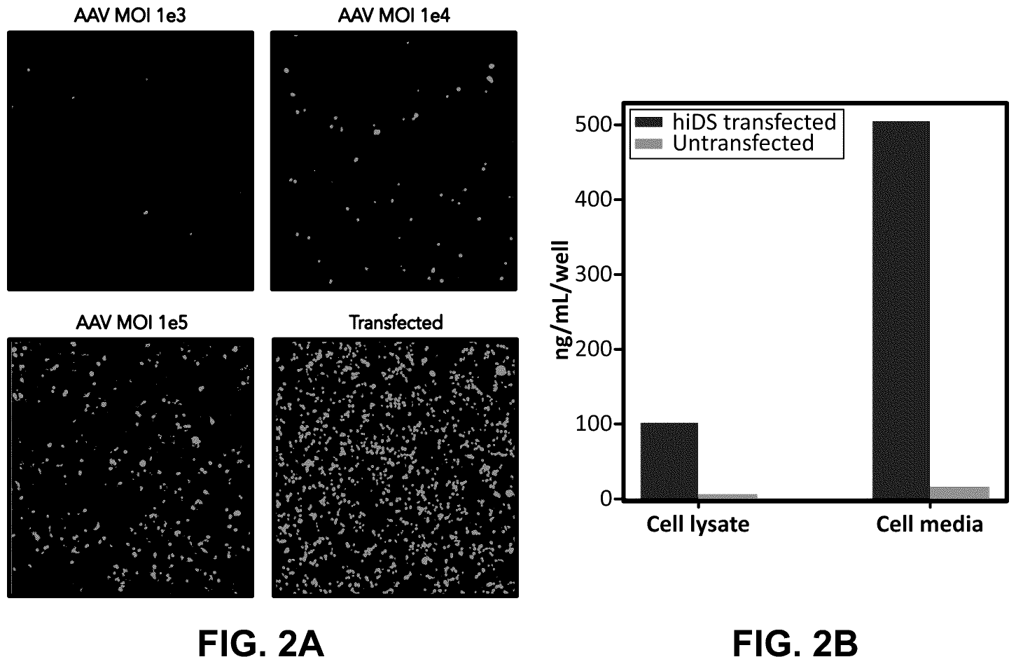

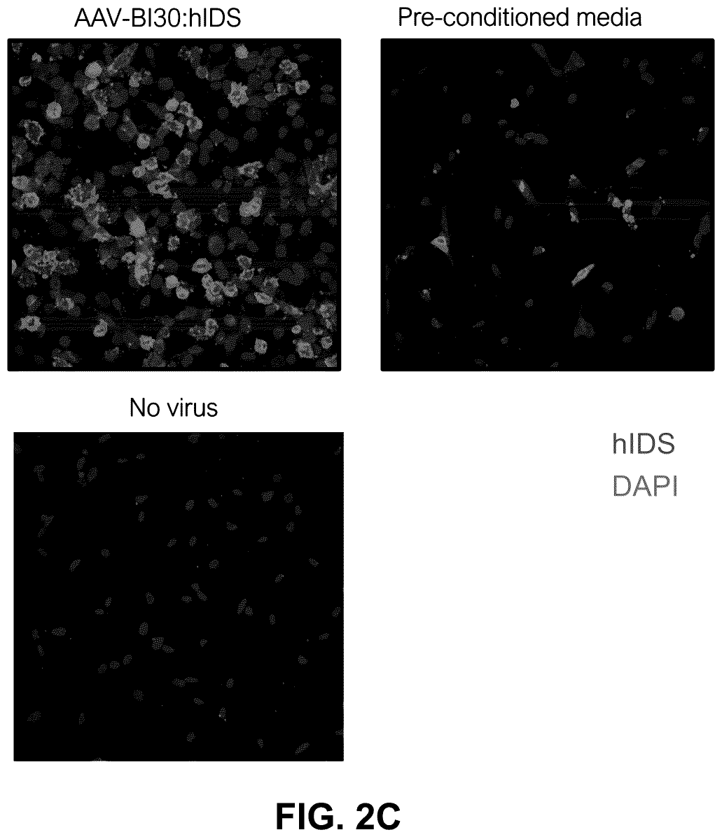

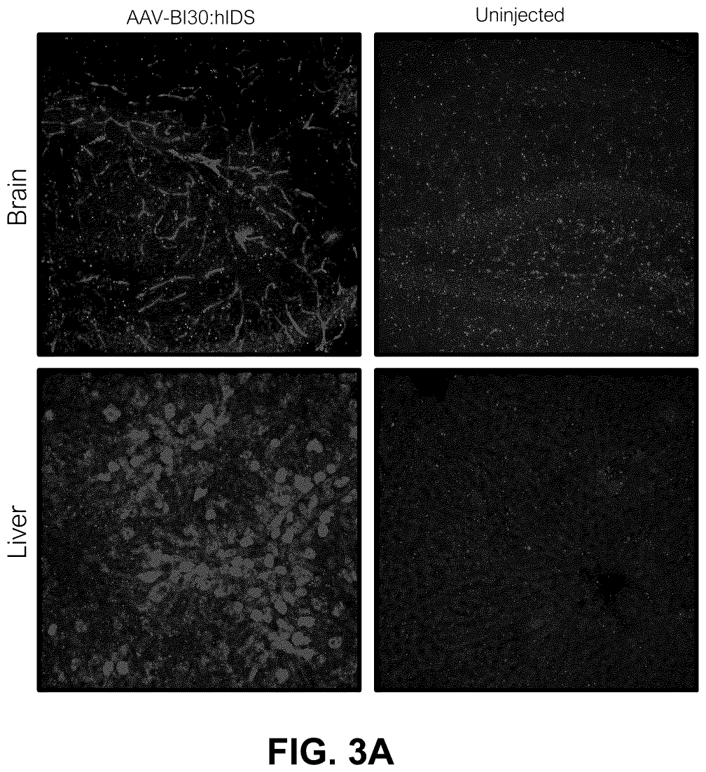

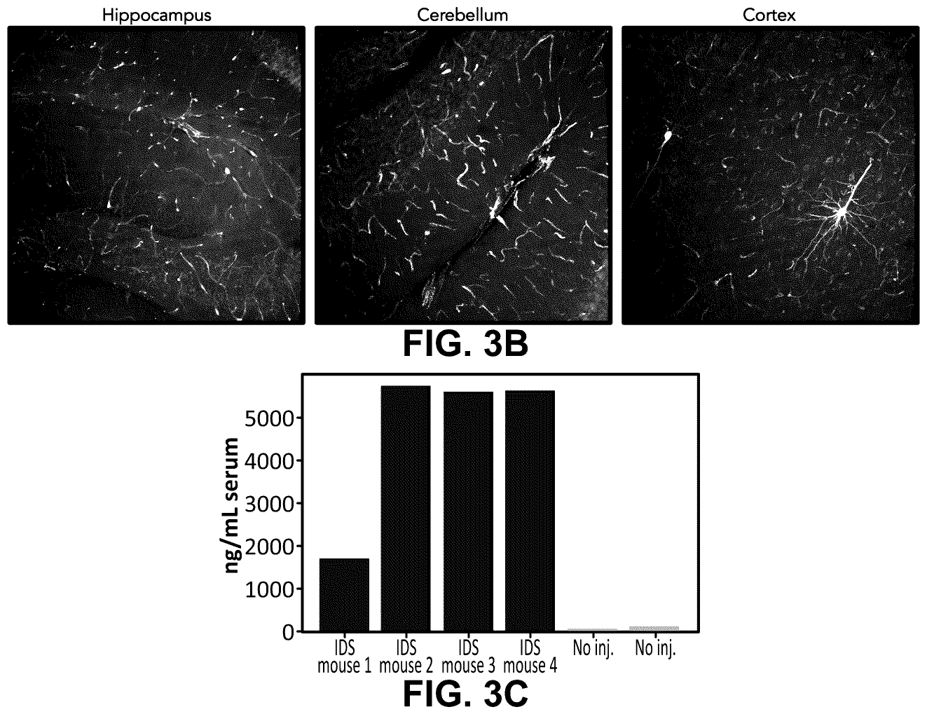

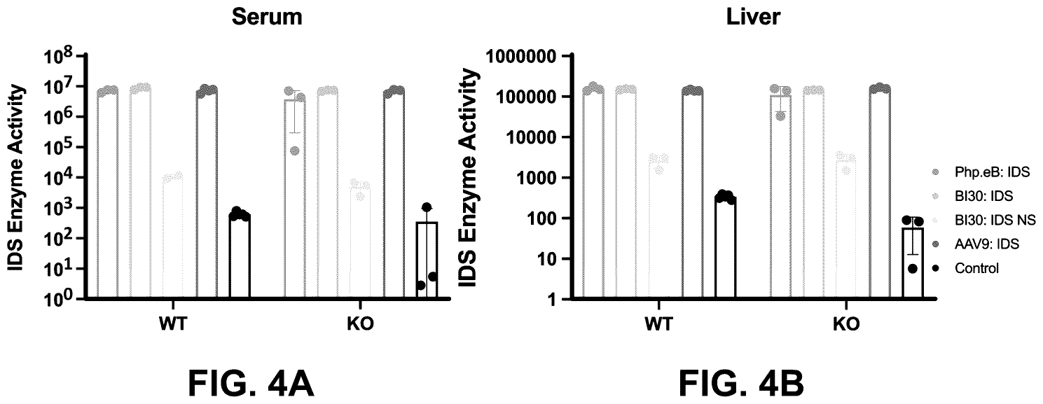

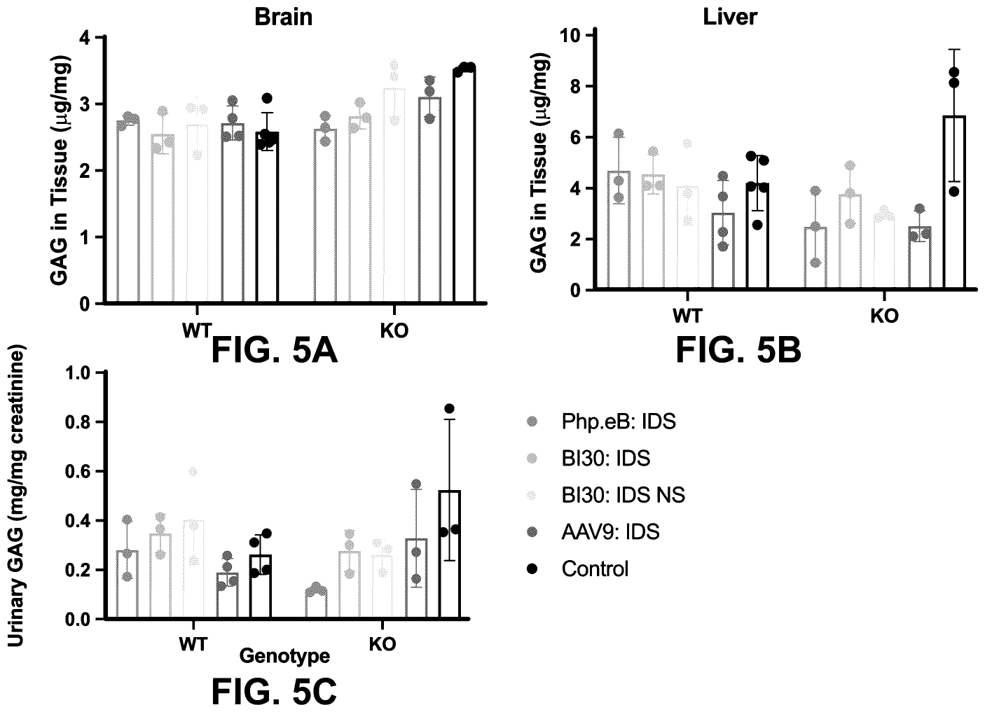

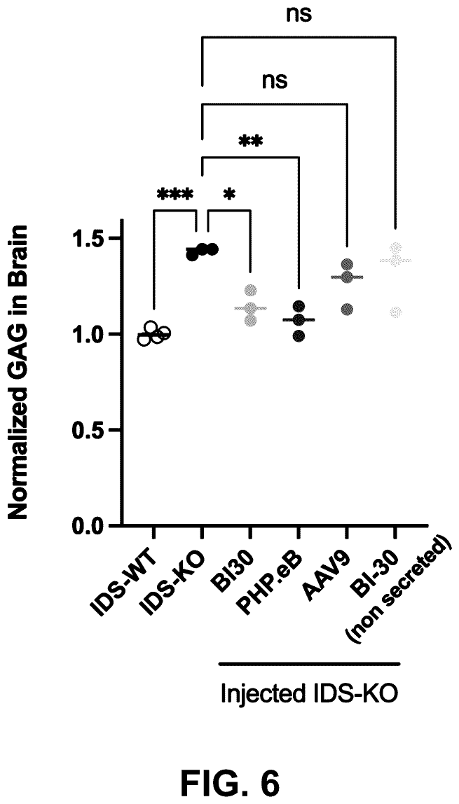

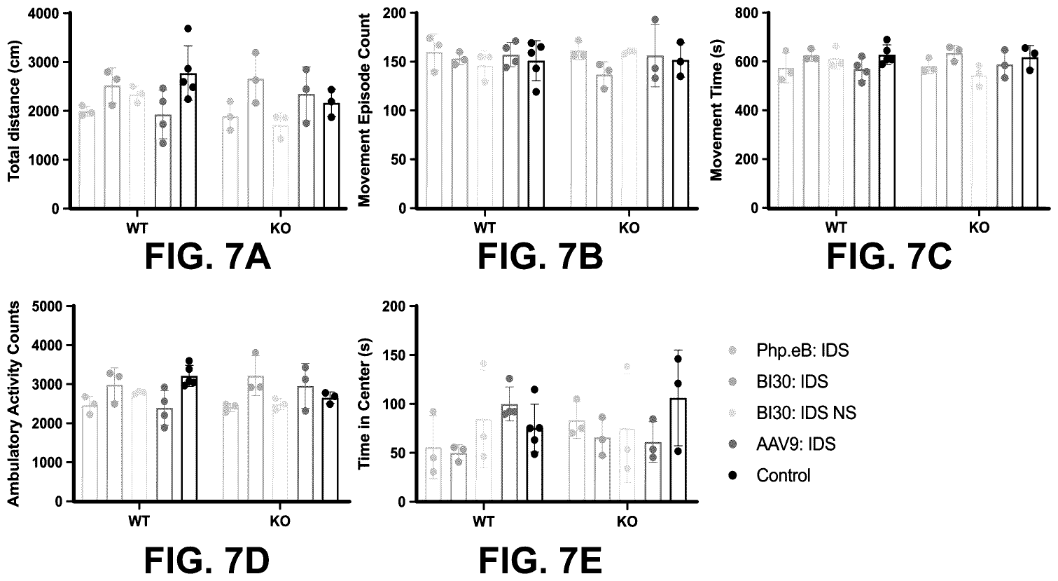

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M.J. MacPherson, B.D. Hames, and G. R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Patent Publication WO 2020/160337 filed Jan. 30, 2020, the contents of which are incorporated specifically herein by reference.

All publications, published patent documents, and patent applications cited herein are hereby incorporated by reference to the same extent as though each individual publication, published patent document, or patent application was specifically and individually indicated as being incorporated by reference.

### OVERVIEW

Gene therapy that can directly target the CNS is a promising therapeutic strategy for targeting LSDs and neurogenetic disorders. Gene therapy refers to the introduction of genes into cells in place of defective or deficient genes that cause disease. Embodiments disclosed herein propose the use of AAV-mediated gene expression in the brain endothelium as a strategy to address neurological LSDs. Without being bound to a particular theory, this approach may rely on the phenomenon of cross-correction, the process through which transduced cells can secrete enzymes to be endocytosed by non-transduced cells.

Cross-correction of enzymes from brain endothelial cells across the BBB to cells in the CNS is a promising approach to gene therapy for LSDs with neurological involvement. The recently described AAV capsid BI30 efficiently and specifically transduces endothelial cells throughout the mouse and rat CNS as well as in cultured human endothelial cell lines (See e.g., the published International Patent Application No. WO2013/004367A2 and Krolak, 2022). Embodiments disclosed herein are direct to therapeutic compositions comprising said CNS-specific AAVs and a transgene for treating or ameliorating the symptoms of LSD and methods of use thereof.

Embodiments disclosed herein provide methods of treating a subject at risk for, or suffering from, a LSD comprising administering to a subject in need thereof, a therapeutically effective amount of one or more compositions comprising a cargo capable of treating the LSD and a targeting moiety having an enhanced selectivity for endothelial cells of the central nervous system (CNS) vasculature, including spinal and retinal vasculature. LSDs result from defective lysosomes comprising inborn metabolism errors from defects in single genes. The accumulation of defective lysosomes results in dysfunctional organs and result in morbidity and mortality. LSDs affect multiple organs but certain organs, like the central nervous system (CNS), are more affected in early life. CNS endothelial cells line the luminal face of blood vessels, including the blood-brain-barrier, which orchestrate key homeostatic processes. Situated at the interface of the nervous and circulatory systems, endothelial cells actively regulate the biochemical composition of the CNS microenvironment, the transmission of inflammatory and immune signals and the dynamic coupling of blood flow to meet local neuronal energetic domain. Thus, embodiments disclosed provide a method for a selective and high-efficiency delivery system for cargos capable of treating LSDs to this critical cell and tissue type.

Accordingly, embodiments disclosed herein provide methods of delivering cargos capable of treating a LSD with enhanced selectivity and efficiency to the CNS vasculature. Embodiments disclosed herein also provide vector systems for the generation and loading of such delivery particles with the cargo capable of treating a LSDs. These targeting moieties may be incorporated into particles, such as viral capsid-based delivery particles, to confer a tropism on the delivery particles and enhance transduction of endothelial cells of the CNS vasculature. Likewise, embodiments disclosed herein provide methods for use of such compositions to target CNS endothelial cells, in vitro and in vivo, with implications for both therapeutic and research purposes for the treatment of LSDs.

Additional feature and advantages of the aforementioned embodiments are further described below.

### Methods of Treating Lysosomal Storage Diseases

In one aspect, embodiments disclosed herein are directed to method of treating a subject at risk for, or suffering from, a lysosomal storage disease. In one example embodiment, the method comprises administering a therapeutically effective amount of a recombinant adeno-associated virus (AAV) delivery capsid, the capsid comprising a vector (also referred to as an “artificial genome”) comprising a transgene encoding a polypeptide effective to the LSD. The rAAV comprises one or more modifications that provide the assembled rAAV capsid with a tropism for CNS endothelial cells, which in turn enables efficient ability of the rAAV to cross the blood-brain-barrier.

In example embodiments, a subject at risk for, or suffering from, a LSD are treated by delivering a cargo using the compositions as described herein and/or the vector systems as described herein of a wild-type gene, corresponding to the LSD, to endothelial cells of the CNS vasculature, i.e., increasing expression of a wild-type copy of said gene to restore normal levels of these critical gene products in the vascular endothelial cells of the CNS using a gene therapy approach. As used herein, the terms “gene therapy” and “gene delivery” are used interchangeably and refer to modifying or manipulating the expression level of a gene to alter the biological properties of living cells for therapeutic use.

In example embodiments, the gene therapy is performed either ex vivo or in vivo. Ex vivo gene therapy comprises harvesting a subject at risk for, or suffering from, a LSD and transduced with a composition as described herein and/or the vector systems as described herein to deliver a therapeutic gene in vitro. The subject at risk for, or suffering from, a LSD is subsequently infused with the gene-corrected cells. In vivo gene therapy comprises delivering to a subject at risk for, or suffering from, a LSD genetic material according to a systemic or with an intra-parenchymal, in situ administration, to target specific organs and adequate concentrations of the genetic material capable of treating the LSD. While this procedure may appear to inadvertently lead to gene transfer into tissues and cell types that are not targets, compositions as described herein and/or the vector systems as described herein significantly reduces this issue because of the increased transduction of endothelial cells of the CNS vasculature.

For more information regarding gene therapy for LSDs see e.g., Penati R, et al. A. Gene therapy for lysosomal storage disorders: recent advances for metachromatic leukodystrophy and mucopolysaccaridosis I. J Inherit Metab Dis. 2017 July; 40(4):543-554; Cachon-Gonzalez et al. Genetics and Therapies for GM2 Gangliosidosis. Curr Gene Ther. 2018; 18(2):68-89; and Nagree M S, Scalia S, McKillop W M, Medin J A. An update on gene therapy for lysosomal storage disorders. Expert Opin Biol Ther. 2019 July; 19(7):655-670.

CNS Endothelial Cell-Specific rAAV and Therapeutic Compositions Thereof

Described herein are various embodiments of engineered viral capsids, such as adeno-associated virus (AAV) capsids, that can be engineered to confer cell-selective tropism, such as CNS vascular endothelial cell tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-selective tropism to the engineered viral particle. The engineered viral capsids described herein can include one or more engineered viral capsid proteins described herein. The engineered viral capsids described herein can include one or more engineered viral capsid proteins described herein that can contain one or more targeting moieties as described above.

The engineered viral capsids can be variants of wild-type viral capsid. For example, In example embodiments, the engineered AAV capsids can be variants of wild-type AAV capsids. In example embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In example embodiments, the serotype of the reference wild-type AAV capsid can be AAV-1, AAV-2, AAV-3, AAV-4, AAV-5, AAV-6, AAV-8, AAV-9 or any combination thereof. In example embodiments, the serotype of the wild-type AAV capsid can be AAV-9. The engineered AAV capsids can have a different tropism than that of the reference wild-type AAV capsid.

In an example embodiment, the targeting moieties disclosed herein can be inserted between two amino acids in the wild-type viral protein (VP) (or capsid protein). In example embodiments, the n-mer motif can be inserted between two amino acids in a variable amino acid region in a viral capsid protein.

In example embodiments, the n-mer motif can be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parovirus capsids (see e.g., DiMattia et al. 2012. J. Virol. 86(12):6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011.

“Adeno-Associated Virus Biology.” In Snyder, R. O., Moullier, P. (eds.) Totowa, NJ: Humana Press). In one example embodiment, one or more targeting moieties can be inserted between two amino acids in one or more of the 12 variable regions in the wild-type AVV capsid proteins. In one example embodiment, the one or more targeting motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In one example embodiment, the engineered capsid can have a 7-mer motif inserted between amino acids 588 and 589 of an AAV9 viral protein. SEQ ID NO: 1 is a reference AAV9 capsid sequence for at least referencing the insertion sites discussed above. It will be appreciated that targeting moieties can be inserted in analogous positions in AAV viral proteins of other serotypes, such as but not limited to, AAV1, AAV2, AAV3, AAV4, AAV5, AAV6, AAV7, AAV8, AAV9, AAV rh.74, AAV rh.10 capsid polypeptide. In example embodiments as previously discussed, the targeting moieties can be inserted between any two contiguous amino acids within the AAV viral protein and In example embodiments the insertion is made in a variable region.

In one example embodiment, the first 1, 2, 3, or 4 amino acids of an n-mer motif can replace 1, 2, 3, or 4 amino acids of a polypeptide into which it is inserted and preceding the insertion site. Using an AAV as another non-limiting example, one or more of the n-mer motifs can be inserted into e.g., an AAV9 capsid polypeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that In example embodiments, no amino acids in the polypeptide into which the targeting moiety is inserted are replaced by the targeting moiety.

The engineered viral capsid and/or capsid proteins can be encoded by one or more engineered viral capsid polynucleotides. In example embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In example embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The therapeutic composition embodiments include an engineered AAV capsid system, Engineered AAV capsid particles can be used generally to package and/or deliver one or more transgenes capable of treating LSDs to endothelial cells of the CNS vasculature. In example embodiments this is conferred by the tropism of the engineered AAV capsid, which can be influenced at least in part by the inclusion of one or n-mer motifs described elsewhere herein.

### Targeting Moiety

In example embodiments, an engineered cell can be delivered to a subject, where it can release produced compositions of the present invention (including but not limited to engineered AAV capsid particles) such that they can then deliver a cargo (e.g., a cargo polynucleotide(s)) to a recipient cell. These general processes can be used in a variety of ways to treat and/or prevent disease or a symptom thereof in a subject, generate model cells, generate modified organisms, provide cell selection and screening assays, in bioproduction, and in other various applications.

In example embodiments, the compositions, such as polypeptides and other particles (e.g., engineered AAV capsids and viral particles), containing one or more of the targeting moieties can be delivered to endothelial cells of the CNS vasculature.

The targeting moiety comprise a n-mer motif. The n-mer motif may comprise or consists of X1-X2-X3-X4-X5-X6-X7, where position X2 is an N (Asn) and position X5 is either a K (Lys) or R (Arg) and positions X1, X3, X4, X6, and X7 are any amino acid. It should be understood that any reference to any amino acid is intended to encompass any natural amino acid as well as any amino acid mimetic having similar physical and chemical characteristics to naturally occurring amino acids. In one example embodiment, X5 may also be any amino acid mimetic capable of providing a positive a positive charge like that of K or R. In an example embodiment, the composition of the n-mer motif may be selected such that overall charge of the n-mer motif at neutral pH is between 0 and +2.

In one example embodiment, X1, X3, X4, X6, and X7 are independently selected from the following groups: X1 is selected from the group consisting of G, M, T, S, N, D, L, H, P, I, V, Q, Y, W, F, A, E; X3 is selected from the group consisting of amino acids N, S, T, H, D, A, Y, M, Q, E, R, G, V; X4 is selected from the group consisting of T, V, I, A, M, S, H, W, N; X6 is selected from the group consisting of N, S, G, D, P, T, H, Q, A, Y. X7 is selected from the group consisting of T, Y, W, N, V, I, H, M, S, G, A, Q, F, D, P, R, L.

In another example embodiment, the n-mer motif of X1, X3, X4, X6, and X7 are independently selected from the following groups: X1 is selected from the group consisting of G, M, T, S, N, D; X3 is selected from the group consisting of N, S, T, H, D; X4 is selected from the group consisting of T, V, I, A; X6 is selected from the group consisting of N, S, G, D, P; and X7 is selected from the group consisting of T, Y, W, N, V, I, H, M, S, G, A, Q, F, D, P, R, L.

In one example embodiment, if X1 is R or K then at least one of X3, X4, X6 and X7 are D or E.

In an example embodiment, the composition of the n-mer at position X1 is not R, K or C; X3 is not W, F, K, C, I, P, or L; X4 is not Y, G, P, D, C, Q, R, K, E, F, L, or R; X6 is not R, I, W, V, F, C, L, E, or K; and X7 is not C, K, E.

In an example embodiment, the targeting moiety can be further defined by the formula as: X1-N-X3-(T, V, I, A, M, S, H, W, N)-(K, R)-X6-X7, where position X2 is an N, position X4 is either a T, V, I, A, M, S, H, W, or N, position X5 is K or R and positions X1, X3, X6, X7 are any amino acid.

In an embodiment, the targeting moiety is further defined by the formula as: X1-X2-N-X3-(T, V, I, A)-(K, R)-X6-X7, where position X2 is an N, position X4 is either a T, V, I, A, position X5 is either K or R and positions X1, X3, X6, X7 are any amino acid.

In an embodiment, the targeting moiety is further defined by the formula as: X1-X2-N-X3-(T, V, I, A)-(K, R)-X6-X7, where position X2 is N, position X4 is T, V, I, A, position X5 is K or R.

In an example embodiment, the targeting moiety is further defined by: X1 is selected from (G, M, T, S, N, D) or (L, H, P, I, V, Q, Y, W, F, A, E) or (R, K, E, C) or (R, K), X2 is N, wherein at position X3 is either (N, S, T, H, D) or (A, Y, M, Q, E, R, G, V) or (W, F, K, C, I, P, L); at position X4 is either (T, V, I, A) or (M, S, H, W, N) or (Y, G, P, D, C, Q, R, K, E, F, L, R); position X5 is either (K) or (R); position X6, is either (N, S, G, D, P) or (T, H, Q, A, Y) but not (R, I, W, V, F, C, L, E, K); at position X7, is either (T, Y, W, N, V, I, H, M, S, G, A, Q, F, D, P, R, L) but not (C, K, E); and wherein when X1 is R or K, X3, X4, X6 or X7 is selected from D or E.

In one example embodiment, the n-mer is selected from any one of the n-mer motifs as listed in Tables 1-6 below.

In one example embodiments, the targeting motif is NNSTRGG (SEQ ID NO: 1) (BI30). In another example embodiment, the targeting motif is GNSARNI (SEQ ID NO: 2) (BI33). In another example embodiment, the targeting motif is BI55: GNSVRDF (SEQ ID NO: 3).

Example embodiments further include polynucleotides encoding any of the above-mentioned targeting moieties.

Targeting moieties specific for endothelial cells of the CNS vasculature are further described in Published International Patent Application No. WO 2013/004367 A2, herein incorporated by reference in its entirety.

In an embodiment, the targeting moiety can be used to increase transduction in target cells. The increase in transduction efficiency of the targeting moiety to a cell may be compared to a composition that does not contain the targeting moiety, for example inclusion of one or more targeting moieties in a composition can result in an increase in transduction and or transduction efficiency by 10%, 20%, 30%, 40%, 50%, 60%, 70%, 80%, 90%, 100% or more. In an exemplary embodiment, the increase in transduction and or transduction efficiency is two-fold, three-fold, four-fold, five-fold, six-fold, seven-fold, eight-fold, nine-fold, ten-fold, 20-fold, 30-fold, 40-fold, 50-fold, 60-fold, 70-fold, 80-fold or more relative to a composition lacking the targeting moiety. In one embodiment, the transduction and/or transduction efficiency is increased or enhanced in endothelial cells. In one embodiment, there is an increase in endothelial cells of the vasculature, for example, the central nervous system vasculature. In embodiments, the transduction and/or transduction efficiency is increased or enhanced in cells of the central nervous system. In embodiments, the transduction and/or transduction efficiency is increased or enhanced in hepatocytes or in endothelial cells of the kidney or of the muscle. In an embodiment, the composition comprising a targeting moiety is selective to a target cell as compared to other cell types and/or other virus particles. As used herein, ‘selective’ and ‘cell-selective’ refers to preferential targeting for cells as compared to other cell types. Preferably, the targeting moiety is selective for a desired target (e.g., cell, organ, system e.g., large diameter arteries and veins, brain, retina and spinal cord microvasculature, species) or set of targets by at least 2:1, 3:1, 4:1, 5:1, 6:1 7:1, 8:1, 9:1. 10:1 or more; or 10%, 20%, 30%, 40%, 50%, 60%, 70%, 75%, 80%, 85%, 90% or more, relative to other targets or cells (e.g., microvasculature of peripheral organs such as the kidney). In an embodiment, the composition comprising a targeting moiety described herein can have an increased uptake, delivery rate, transduction rate, efficiency, amount, or a combination thereof in a target cell (e.g., endothelial cells across the arterio-venous axis in brain, retina, and spinal cord vasculature) as compared to other cell types (e.g., muscle cells) and/or other virus particles (e.g., AAVs not containing the targeting moiety) and other compositions that do not contain the cell-selective n-mer motif of the present invention.

As discussed above, the targeting moieties of the present invention confer a strong tropism bias for across the arterio-venous axis in brain, retina, and spinal cord vasculature, including arterial, capillary, and venous endothelial cells. However, in certain context, transduction may also occur to a lesser extent in liver hepatocyte, lung microvascular endothelial cells, and the endothelial lining of large arteries and veins through the systemic circulation following intravenous administration. When deployed for research purposes where CNS-selectivity is critical, a Cre-dependent viral genome could be used in tandem with a CNS endothelial cell-selective transgenic driver—such as MFSD2A:Cre^{ERT242} or SLCO1C1:Cre^{ERT2 42} to minimize peripheral transduction. For therapeutic uses, one or more repeat elements may be incorporated in the viral vector systems disclosed herein to reduce non-CNS endothelial vasculature expression. For example, to reduce expression in hepatocytes, repeats of the hepatocyte-selective miR-122 target sequence into the 3′UTR.

### Further Capsid Modifications

In one example embodiment, the viral capsid protein may comprise one or more mutations relative to wild type. In one example embodiment, the one or more mutations comprise K449R in AAV9, or in an analogous position in an AAV1, AAV2, AAV3, AAV4, AAV5, AAV6, AAV7, AAV8, AAV rh.74, AAV rh.10 capsid polypeptide.

### Lysosomal Storage Diseases and Corresponding Transgenes for Delivery

In embodiments, a method is disclosed wherein the disease or disorder is a lysosomal storage disorder, cancer, neurological disorder or infection. LSDs are characterized by abnormalities in one or more components of a lysosome, such as the luminal and membrane-integral proteins, generally caused by a single genetic mutation. These abnormalities may disrupt lysosomal function and contribute to morbidity and mortality.

Generally, the abnormalities result from enzyme dysfunction (i.e., ˜70%), while the remaining abnormalities result from dysfunction of enzyme activators or associated proteins such as a membrane transporter or a membrane protein. These dysfunctions typically are caused by single-gene defects. Transcription of these single gene defects results in inactive enzymes, defective activators, or defective associated proteins.

LSD defective enzymes can result from different types of mutations. For example, defective enzymes can result from nonsense or missense mutations. A nonsense mutation generally will produce a greater defect than a missense mutation. A single disease may have gene variations numbering up to a thousand. In some instances, mutations belong to a family and this information can be used to screen for particular genetic defects. Furthermore, genotype-phenotype correlations may be predictable in some instances.

LSDs may appear uncommon however when grouped approximately 1 in 5000 to 1 in 8000 people suffer from LSDs. Moreover, it may appear LSDs are spread across the general populations however people of specific ethnic and/geographical backgrounds suffer more from particular LSDs. For example, Eastern European Jews (Ashkenazi Jews) suffer from Gaucher disease as high as 1 in 800 and Tay-Sachs disease as high as 1 in 3900, Niemann-Pick A and mucolipidosis IV also occur more frequently in this population. In the Finnish population, aspartylglucosaminuria occurs at a frequency of 1 in 18500 and Salla disease is also common in this population. While LSDs may present in different stages of life (e.g., prenatal, childhood, adulthood) infants suffer much more from LSDs than adults.

In embodiments, a method is disclosed wherein the subject suffers from a lysosomal storage disease and the composition or vector is configured to deliver an enzyme missing in the lysosomal storage disease, a therapeutic polynucleotide encoding the enzyme, or a therapeutic polynucleotide encoding the defective gene to endothelial cells of the CNS vasculature. In embodiments, a method is disclosed wherein the lysosomal storage disease is under the classification of Mucopolysaccharidosis (MPS), Sphingolipidosis, Oligosaccharidosis, Neuronal ceroid lipofuscinosis, Sialic acid disorders, Mucolipidosis, Lysosomal Acid lipase deficiency infantile and childhood/adult types, Pompe disease, Danon disease, Wolman's disease and Cystinosis.

In example embodiments, the MPS is selected from the group consisting of Hurler syndrome (MPS I), Hunter syndrome (MPS II), San Filippo syndrome A (MPS IIIA), San Filippo B (MPS IIIB), San Filippo C (MPS IIIC), San Filippo D (MPS IIID), Morquio syndrome A (MPS IVA), Morquio syndrome B (MPS IVB), Scheie syndrome (MPS V), Maroteaux-Lamy syndrome (MPS VI), Sly syndrome (MPS VII), hyaluronidase deficiency (MPS IX), and Hurler-Scheie syndrome.

In example embodiments, the Sphingolipidosis is selected from the group consisting of GM2 gangliosidosis Type A (Tay-Sachs disease), GM2 gangliosidosis Type O (Sandhoff disease), GM2 gangliosidosis Type AB (GM2 activator deficiency), Niemann-Pick disease A, Niemann-Pick disease B, Niemann-Pick disease C, Gaucher disease Type 1, Gaucher disease Type 2, Gaucher disease Type 3, Fabry disease, Farber's disease, Metachromatic leukodystrophy, Globoid leukodystrophy (Krabbe' disease), GM1 gangliosidosis Type 1, GM1 gangliosidosis Type 2, GM1 gangliosidosis Type 3, and Multiple sulfatase deficiency.

In example embodiments, the Oligosaccharidosis is selected from the group consisting of alfa-mannosidosis, beta-mannosidosis, Schindler disease, Aspartylglucosaminuria, and Fucosidosis.

In example embodiments, the Neuronal ceroid lipofuscinosis is selected from the group consisting of CLN 1, CLN 2, CLN 3, CLN 4, CLN 5, CLN 6, CLN 7, CLN 8, CLN 9, CLN 10, CLN 11, CLN 12, CLN 13, and CLN 14.

In example embodiments, the Sialic acid disorder is selected from the group consisting of Galactosialidosis, Infantile sialic acid storage disease, Salla disease, and Sialuria.

In example embodiments, the Mucolipidosis is selected from the group consisting of Sialidosis I and II (Mucolipidosis I), I-cell disease (Mucolipidosis II), Pseudo-Hurler-Polydystrophy (Mucolipidosis III), and Mucolipidosis IV.

The above LSDs that can be treated in accordance with the methods and compositions described herein. Further information regarding LSDs may be found in Rajkumar V, Dumpa V. Lysosomal Storage Disease. [Updated 2022 Jul. 25]. In: StatPearls [Internet]. Treasure Island (FL): StatPearls Publishing; 2022 January-, incorporated herein by reference. Further lysosomal storage diseases are described in Table 1 of Platt et al., (2012) J. Cell. Biol. Col. 199, no. 5 723-734, incorporated herein by reference. Methods as detailed herein may be used with additional therapies for lysosomal storage diseases, exemplary therapies are described in Table 2 of Platt et al, incorporated herein by reference.

Metabolic disorders of the brain that manifest in the neonatal or early infantile period are usually associated with acute and severe illness and are thus referred to as devastating metabolic disorders. Most of these disorders may be classified as organic acid disorders, amino acid metabolism disorders, primary lactic acidosis, fatty acid oxidation disorders and nutrient transport disorders. Each disorder has distinctive clinical, biochemical, and radiologic features. Early diagnosis is important both for prompt treatment to prevent death or serious sequelae and for genetic counseling. However, diagnosis is often challenging because many findings overlap and may mimic those of more common neonatal conditions, such as hypoxic-ischemic encephalopathy and infection. If one of these rare disorders is suspected, the appropriate biochemical test or analysis of the specific gene should be performed to confirm the diagnosis.

### Example Transgenes

In example embodiments, the composition delivered to treat LSD is a transgene, also referred to as a therapeutic transgene. In example embodiments, the transgene is engineered from a natural gene such as those described herein. In example embodiments, the transgene is codon optimized. Generally, the genomic cDNA of the subject is used to design codon usage bias.

In another example embodiment, gene therapy approaches may be used to deliver endothelial cells to produce and secrete gene products such as lysosomal enzymes or antibodies, for treating lysosomal storage diseases.

Only ˜145 bp AAV ITRs are required for recombinant AAV (rAAV) propagation because they participate in vector production, induce transgene expression, and ensure continual cell transduction. Accordingly, ˜96% of the AAV genome can be removed for gene therapy. For example, the rep and cap genes can be substituted for the expression cassette containing a promoter (such as those described herein), a therapeutic transgene (for example, IDS) and a poly (A) tail forms the essence of all AAV vectors.

In example embodiments, additional modifications may be implemented to further increase the efficacy of the AAV. For example, the AAV ITRs may be modified to increase the expression of the rAAV vector upon transduction, which may allow the transgene to be expressed without second-strand DNA synthesis; the promoter may be modified to increase transcription; and the codons in the transgene may be engineered to modify mRNA production and/or translation.

In example embodiments, the ITRs are modified to overcome second-strand synthesis after infection. The AAV transduction rate is restricted by the synthesis of dsDNA from the single-stranded AAV genome. ITRs initiate second-strand synthesis. In an example embodiment, modified ITRs are no longer suitable substrates for the Rep68 and Rep78 proteins. As a result, the terminal resolution of replication is obviated, and specific self-complementary AAV (scAAV) replication intermediates are produced. The scAAV intermediates comprise plus and minus strands of DNA fused by the modified ITRs encapsulated into the virion shell. Wild-type AAVs package either a single plus-strand or minus-strand DNA. The modified scAAV intermediates are delivered to the nucleus, these plus and minus strands instantaneously anneal to form dsDNA.

In example embodiments, the cis-elements are optimized for targeted delivery. The cis-elements are optimized because the packaging capacity of AAVs is restricted. In an example embodiment, small cis-elements replace long promoter sequences for the delivery of large therapeutic transgenes (e.g., 4.4-4.5 kbs).

In example embodiments, several strategies may be used to deliver transgenes using AAV vectors. Example approach 1 takes advantage of AAV genome is concatemerization via the homologous recombination of ITR sequences. In this approach, transgene cassettes may be split into two or more vectors, which are then delivered to the same cells. After the virus is uncoated, an intact transgene is formed by the homologous recombination between the two or more fragments.

In example approach 2, truncated transgene fragments of different lengths are packaged into different AAV virions at undefined locations on the vector genome. Either homologous recombination of the overlapping regions of the different AAV vector genomes or annealing of different AAV vector genomes at complementary regions via single-stranded templates produces the transgene cassette. In example embodiments, overlapping fragments may be added to the end of the individual AAV vectors to encourage homologous recombination.

In example approach 3, a hybrid dual-vector incorporates an overlapping region with intron splice sites in the split vector transgenes. Approach 3 uses concatemerization activity of AAV genomes to bring independent AAV vector genomes together. Recombination (for example, the starting vectors are segregated into two halves each carrying the 5′ and 3′ splicing elements, respectively), and splicing provide the appropriate transgene protein. This strategy may increase the expression of full functional protein.

In example approach 4, an AAV genome is cross-packaged into the capsids of other parvoviruses thus creating chimeric vectors. In example approach 5, intein-mediated protein trans-splicing is used. Intein catalyzes protein splicing thereby causing the ligation of two polypeptides via trans-splicing (this approach is similar to intron-mediated RNA splicing). Multiple AAV vectors are delivered to the same cells. Each of the AAV vectors encode one of the fragments of target proteins, the fragments are flanked by short split inteins. The full-length protein forms after protein trans-splicing. See e.g., Li, C., Samulski, R.J. Engineering adeno-associated virus vectors for gene therapy. Nat Rev Genet 21, 255-272 (2020), herein incorporated by reference.

TABLE I
lists LSDs and their associated genes. The table
is referenced from Rajkumar V, et al. 2022 Tables
A and Table B, herein incorporated by reference.

| LSD | Gene | LSD | Gene |
|---|---|---|---|
| Gangliosidosis | GLB 1 | Krabbe's Disease | GALC |
| Metachromatic | ASA | Fabry's Disease | GLA |
| leukodystrophy | | | |
| Gaucher Disease | GBA | Niemann-Pick Disease A | SMPD1 |
| Niemann-Pick | SMPD1 | Niemann-Pick Disease C1 | NPC1 |
| Disease B | | | |
| Niemann-Pick | NPC2 | Tay-Sachs Disease | HEXA |
| Disease C2 | | | |
| Sandhoff Disease | HEXB | GM2-Activator Deficiency | GMSA |
| Multiple | SUMF1 | Alpha Mannosidosis | MAN2B1 |
| Sulfatase | | | |
| Deficiency | | | |
| Schindler's | NAGA | Aspartylglucosaminuria | AGA |
| Disease | | | |
| Fucosidosis | FUCA1 | Hurler Syndrome | IDUA |
| Scheie Syndrome | IDUA | Hurler-Scheie Syndrome | IDUA |
| Hunter syndrome | IDS | San Filippo Syndrome A | SGSH |
| San Filippo | NAGLU | San Filippo Syndrome C | HGSNAT |
| Syndrome B | | | |
| San Filippo | GNS | Morquio Syndrome A | GALNS |
| Syndrome D | | | |
| Morquio | GLB1 | Maroteaux-Lamy Syndrome | Aryl |
| Syndrome B | | | sulfatase B |
| Sly Syndrome | GUSB | Neuronal Ceroid | CLN1- |
| | | Lipofuscinosis | CLN14 |
| Galactosialidosis | CTSA | Infantile Sialic | SLC17A5 |
| | | Acid Storage Disease | |
| Salla Disease | SLC17A5 | Sialuria | GNE |
| Sialidosis I and II | NEU1 | I-cell Disease | GNPTAB |
| Pseudo-Hurler- | GNPTAB | Mucolipidosis IV | MCOLN1 |
| Polydystrophy | | | |
| Lysosomal Acid | LIPA | Pompe's Disease | GAA |
| Lipase | | | |
| Deficiency | | | |
| Danon Disease | LAMP2 | Cystinosis | CTNS |

### Example Treatment of MPS II with IDS Gene Therapy

In example embodiments, methods and compositions described herein are used to treat a subject at risk for or suffering from Mucopolysaccharidosis Type II (MPS II), also known as Hunter syndrome. MPS II is a X-linked recessive disorder involving multiple organ systems and appears at an early age. The severity of MPS II is dependent on the phenotype with certain phenotypes leading to high morbidity and mortality.

MPS II results from a deficiency of the iduronate 2-sulfatase enzyme (IDS) and is an X-linked recessive disorder that, in general, occurs in males. IDS breaks down glycosaminoglycans and decreased activity of IDS leads to accumulation of heparan sulfate (HS) and dermatan sulfate (DS) in multiple organs both intra- and extracellularly. IDS is located on the Xq28 chromosome and encodes the enzyme iduronate 2-sulfatase, which is found in lysosomes. This enzyme participates in the breakdown of glycosaminoglycans (GAGs). 640 mutations of this gene are related to Hunter Syndrome and can be found in the Human Gene Mutation Database (Stenson P. D., et al. (2003). Human Gene Mutation Database (HGMD): 2003 update. Hum. Mutation. 21, 577-581). The known mutations comprise 323 missense or nonsense, 59 splicing substitutions, 119 small deletions, 49 small insertions/duplications, 14 small indels, 52 gross deletions, 4 gross insertions/duplications, and 20 complex rearrangements. See e.g., Hashmi M S, Gupta V. Mucopolysaccharidosis Type II. [Updated 2022 Aug. 1]. In: StatPearls [Internet]. Treasure Island (FL): StatPearls Publishing; 2022 January-.

In example embodiments, the trans gene delivered by the AAVs disclosed herein comprises a transgene encoding IDS. In one example embodiment, the polynucleotide sequence included in the AAV vector is a DNA sequence derived from the primary accession number NG_011900.3. In another example embodiment, the DNA sequence is NG_011900.3. In example embodiments, the IDS gene is derived from a genomic sequence with accession numbers AC233288.1, AC244197.3, AF011889.1, AH000819.2, AH002847.2, AH005495.2, CH471171.2, EAW61281.1, EAW61282.1, EAW61283.1, EAW61284.1, EAW61285.1, CP068255.2, HH961810.1, JC013507.1, KT724868.1, KY561588.1, KY561589.1, L36845.1, and MF431729.1. In example embodiments, the IDS genomic sequence is selected from the group consisting of AC233288.1, AC244197.3, AF011889.1, AH000819.2, AH002847.2, AH005495.2, CH471171.2, EAW61281.1, EAW61282.1, EAW61283.1, EAW61284.1, EAW61285.1, CP068255.2, HH961810.1, JC013507.1, KT724868.1, KY561588.1, KY561589.1, L36845.1, and MF431729.1.

In example embodiments, the polynucleotide sequence included in the AAV vector is a RNA sequence derived from NM_000202.8, NM_001166550.4, NM_006123.5, NR_104128.2. In example embodiments, the polynucleotide sequence included in the AAV vector is NM_000202.8, NM_001166550.4, NM_006123.5, NR_104128.2. In another example embodiment, the sequence included in the AAV vector is derived from mRNA with the accession numbers: AF050145.1, AI242974.1, AK055600.1, AK057191.1, AK294541.1, AK295815.1, AL117536.1, BC006170.1, BX647357.1, and BY996633.1. In another example embodiment, the sequence included in the vector is a mRNA sequence selected from the group consisting of: AF050145.1, AI242974.1, AK055600.1, AK057191.1, AK294541.1, AK295815.1, AL117536.1, BC006170.1, BX647357.1, and BY996633.1.

In example embodiments, a method is disclosed wherein the subject suffers from Hunter Syndrome, and the compositions or vectors described herein are configured to deliver a wild-type cargo of IDS, or a polynucleotide encoding IDS (i.e., a wild-type cargo of IDS), to vascular endothelial cells of the CNS.

The above is provided as a non-limiting example. Further contemplated is the use of the rAAVs disclosed herein to similarity deliver the other aforementioned transgene for their use in treating their associate LDSs.

### Engineered Vectors and Vector Systems

Engineered vectors of the present invention comprise vectors for generating the engineered capsid proteins which assemble into the rAAV capsid and vectors (also referred to as artificial genome) that encode the transgene to be delivered and that are encapsulated with the formed rAAV capsid.

### Vectors Encoding the Transgene

In one example embodiment, the vector encoding the transgene (also referred to as “an artificial genome”) comprises the transgene to be delivered flanked on either side by AAV ITRs. Only ˜145 bp AAV ITRs are required for recombinant AAV (rAAV) propagation because they participate in vector production, induce transgene expression, and ensure continual cell transduction. Accordingly, ˜96% of the AAV genome can be removed for gene therapy. For example, the rep and cap genes can be substituted for the expression cassette containing a promoter (such as those described herein), a therapeutic transgene (for example, IDS) and a poly (A) tail forms the essence of all AAV vectors.

In example embodiments, additional modifications may be implemented to further increase the efficacy of the AAV. For example, the AAV ITRs may be modified to increase the expression of the rAAV vector upon transduction, which may allow the transgene to be expressed without second-strand DNA synthesis; the promoter may be modified to increase transcription; and the codons in the transgene may be engineered to modify mRNA production and/or translation.

In example embodiments, the ITRs are modified to overcome second-strand synthesis after infection. The AAV transduction rate is restricted by the synthesis of dsDNA from the single-stranded AAV genome. ITRs initiate second-strand synthesis. In an example embodiment, modified ITRs are no longer suitable substrates for the Rep68 and Rep78 proteins. As a result, the terminal resolution of replication is obviated, and specific self-complementary AAV (scAAV) replication intermediates are produced. The scAAV intermediates comprise plus and minus strands of DNA fused by the modified ITRs encapsulated into the virion shell. Wild-type AAVs package either a single plus-strand or minus-strand DNA. The modified scAAV intermediates are delivered to the nucleus, these plus and minus strands instantaneously anneal to form dsDNA.

In example embodiments, the cis-elements are optimized for targeted delivery. The cis-elements are optimized because the packaging capacity of AAVs is restricted. In an example embodiment, small cis-elements replace long promoter sequences for the delivery of large therapeutic transgenes (e.g., 4.4-4.5 kbs).

In example embodiments, several strategies may be used to deliver transgenes using AAV vectors. Example approach 1 takes advantage of AAV genome is concatemerization via the homologous recombination of ITR sequences. In this approach, transgene cassettes may be split into two or more vectors, which are then delivered to the same cells. After the virus is uncoated, an intact transgene is formed by the homologous recombination between the two or more fragments.

In example approach 2, truncated transgene fragments of different lengths are packaged into different AAV virions at undefined locations on the vector genome. Either homologous recombination of the overlapping regions of the different AAV vector genomes or annealing of different AAV vector genomes at complementary regions via single-stranded templates produces the transgene cassette. In example embodiments, overlapping fragments may be added to the end of the individual AAV vectors to encourage homologous recombination.

In example approach 3, a hybrid dual-vector incorporates an overlapping region with intron splice sites in the split vector transgenes. Approach 3 uses concatemerization activity of AAV genomes to bring independent AAV vector genomes together. Recombination (for example, the starting vectors are segregated into two halves each carrying the 5′ and 3′ splicing elements, respectively), and splicing provide the appropriate transgene protein. This strategy may increase the expression of full functional protein.

In example approach 4, an AAV genome is cross-packaged into the capsids of other parvoviruses thus creating chimeric vectors. In example approach 5, intein-mediated protein trans-splicing is used. Intein catalyzes protein splicing thereby causing the ligation of two polypeptides via trans-splicing (this approach is similar to intron-mediated RNA splicing). Multiple AAV vectors are delivered to the same cells. Each of the AAV vectors encode one of the fragments of target proteins, the fragments are flanked by short split inteins. The full-length protein forms after protein trans-splicing. See e.g., Li, C., Samulski, R. J. Engineering adeno-associated virus vectors for gene therapy. Nat Rev Genet 21, 255-272 (2020), herein incorporated by reference.

Vectors for Generating rAAVs

Also provided herein are vectors and vector systems that can contain one or more of the engineered polynucleotides described herein that can encode one or more of the n-mer motifs of the present invention, including but not limited to, engineered viral polynucleotides (e.g., engineered AAV polynucleotides). As used in this context, engineered viral capsid polynucleotides refers to any one or more of the polynucleotides described herein capable of encoding an engineered viral capsid as described elsewhere herein and/or polynucleotide(s) capable of encoding one or more engineered viral capsid proteins described elsewhere herein. Further, where the vector includes an engineered viral capsid polynucleotide described herein, the vector can also be referred to and considered an engineered vector or system thereof although not specifically noted as such. In embodiments, the vector can contain one or more polynucleotides encoding one or more elements of an engineered viral capsid described herein. The vectors and systems thereof can be useful in producing bacterial, fungal, yeast, plant cells, animal cells, and transgenic animals that can express one or more components of the engineered viral capsid, particle, or other compositions described herein. Within the scope of this disclosure are vectors containing one or more of the polynucleotide sequences described herein. One or more of the polynucleotides that are part of the engineered viral capsid and system thereof described herein can be included in a vector or vector system.

In example embodiments, a vector used in the production of the rAAVs disclosed herein comprises a rep gene and cap gene). The rep gene typically encodes Rep78, Rep68, Rep52 and Rep40 from a single ORF. These replication factors aid AAV genome replication and virion assembly. The cap gene typically encodes the three capsid proteins (i.e., virion protein 1 (VP1), VP2 and VP3) from a single ORF as well. In addition, the three capsid proteins are regulated by transcription from a start codon (ACG) and alternative splicing. The cap gene also encodes, from an in-frameshifted ORF, an assembly-activating protein (AAP). The AAP is essential for capsid assembly.

In example embodiments, the vector can include an engineered viral (e.g., AAV) capsid polynucleotide having a 3′ polyadenylation signal. In example embodiments, the 3′ polyadenylation is an SV40 polyadenylation signal. In example embodiments, the vector does not have splice regulatory elements. In example embodiments, the vector includes one or more minimal splice regulatory elements. In example embodiments, the vector can further include a modified splice regulatory element, wherein the modification inactivates the splice regulatory element. In example embodiments, the modified splice regulatory element is a polynucleotide sequence sufficient to induce splicing, between a rep protein polynucleotide and the engineered viral (e.g., AAV) capsid protein variant polynucleotide. In example embodiments, the polynucleotide sequence can be sufficient to induce splicing is a splice acceptor or a splice donor. In example embodiments, the viral (e.g., AAV) capsid polynucleotide is an engineered viral (e.g., AAV) capsid polynucleotide as described elsewhere herein. In example embodiments, the vector does not include one or more minimal splice regulatory elements, modified splice regulatory agent, splice acceptor, and/or splice donor.

The vectors and/or vector systems can be used, for example, to express one or more of the engineered viral (e.g., AAV) capsid and/or other polynucleotides in a cell, such as a producer cell, to produce engineered viral (e.g., AAV) particles and/or other compositions (e.g., polypeptides, particles, etc.) containing an engineered viral (e.g., AAV) capsid or other composition containing an n-mer motif of the present invention described elsewhere herein. Other uses for the vectors and vector systems described herein are also within the scope of this disclosure. In general, and throughout this specification, the term is a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include adeno-associated viruses, and types of such vectors can also be selected for targeting particular types of cells, such as those engineered viral (e.g., AAV) vectors containing an engineered viral (e.g., AAV) capsid polynucleotide with a desired cell-selective tropism. These and other embodiments of the vectors and vector systems are described elsewhere herein.

In example embodiments, the vector can be a bicistronic vector. In example embodiments, a bicistronic vector can be used for one or more elements of the engineered viral (e.g., AAV) capsid system described herein. In example embodiments, expression of elements of the engineered viral (e.g., AAV) capsid system described herein can be driven by a suitable constitutive or tissue specific promoter. Where the element of the engineered viral (e.g., AAV) capsid system is an RNA, its expression can be driven by a Pol III promoter, such as a U6 promoter. In example embodiments, the two are combined.

### Cell-Based Vector Amplification and Expression

Vectors can be designed for expression of one or more elements of the engineered viral (e.g., AAV) capsid system or other compositions containing a target motif of the present invention described herein (e.g., nucleic acid transcripts, proteins, enzymes, and combinations thereof) in a suitable host cell. In example embodiments, the suitable host cell is a prokaryotic cell. Suitable host cells include, but are not limited to, bacterial cells, yeast cells, insect cells, and mammalian cells. The vectors can be viral-based or non-viral based. In example embodiments, the suitable host cell is a eukaryotic cell. In example embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In example embodiments, the host cell is a suitable insect cell. Suitable insect cells include those from Spodoptera frugiperda. Suitable strains of S. frugiperda cells include, but are not limited to, Sf9 and Sf21. In example embodiments, the host cell is a suitable yeast cell. In example embodiments, the yeast cell can be from Saccharomyces cerevisiae. In example embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In example embodiments, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (In Vitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2u plasmids, yeast integrative plasmids, yeast replicative plasmids, shuttle vectors, and episomal plasmids.

In example embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In example embodiments, the vector is a mammalian expression vector. In example embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In example embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the a-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In example embodiments, a regulatory element can be operably linked to one or more elements of an engineered AAV capsid system so as to drive expression of the one or more elements of the engineered AAV capsid system described herein.

Vectors may be introduced and propagated in a prokaryote or prokaryotic cell. In example embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In example embodiments, a prokaryote is used to amplify copies of a vector and express one or more nucleic acids, such as to provide a source of one or more proteins for delivery to a host cell or host organism.

In example embodiments, the vector can be a fusion vector or fusion expression vector. In example embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In example embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In example embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In example embodiments, one or more vectors driving expression of one or more elements of an engineered viral (e.g., AAV) capsid system or other composition containing an n-mer motif described herein are introduced into a host cell such that expression of the elements of the engineered delivery system described herein direct formation of an engineered viral (e.g., AAV) capsid system or other composition containing an n-mer motif described herein (including but not limited to an engineered gene transfer agent particle, which is described in greater detail elsewhere herein). For example, different elements of the engineered viral (e.g., AAV) capsid system or other composition containing an n-mer motif described herein can each be operably linked to separate regulatory elements on separate vectors. RNA(s) of different elements of the engineered delivery system described herein can be delivered to an animal or mammal or cell thereof to produce an animal or mammal or cell thereof that constitutively or inducibly or conditionally expresses different elements of the engineered viral (e.g., AAV) capsid system or other composition containing an n-mer motif described herein that incorporates one or more elements of the engineered viral (e.g., AAV) capsid system or other composition containing an n-mer motif described herein or contains one or more cells that incorporates and/or expresses one or more elements of the engineered viral (e.g., AAV) capsid system or other composition containing an n-mer motif described herein.

In example embodiments, two or more of the elements expressed from the same or different regulatory element(s), can be combined in a single vector, with one or more additional vectors providing any components of the system not included in the first vector. Engineered polynucleotides of the present invention that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In example embodiments, a single promoter drives expression of a transcript encoding one or more engineered viral (e.g., AAV) capsid proteins or other composition containing an n-mer motif described herein, embedded within one or more intron sequences (e.g., each in a different intron, two or more in at least one intron, or all in a single intron). In example embodiments, the engineered polynucleotides of the present invention (including but not limited to engineered viral polynucleotides) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or vectors thereof described herein (including, but not limited to, the engineered AAV capsid polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, brain), or particular cell types (e.g., lymphocytes). Regulatory elements may also direct expression in a temporal-dependent manner, such as in a cell-cycle dependent or developmental stage-dependent manner, which may or may not also be tissue or cell-type specific. In example embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In example embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In example embodiments, the vector can contain a minimal promoter. In example embodiments, the minimal promoter is the Mecp2 promoter, tRNA promoter, or U6. In a further embodiment, the minimal promoter is tissue specific. In example embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In example embodiments a constitutive promoter may be employed. Suitable constitutive promoters for mammalian cells are generally known in the art and include, but are not limited to SV40, CAG, CMV, EF-1a, B-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In example embodiments, the regulatory element can be a regulated promoter. “Regulated promoter” refers to promoters that direct gene expression not constitutively but in a temporally- and/or spatially-regulated manner and includes tissue-specific, tissue-preferred, and inducible promoters. In example embodiments, the regulated promoter is a tissue-specific promoter, as previously discussed elsewhere herein. Regulated promoters include conditional promoters and inducible promoters. In example embodiments, conditional promoters can be employed to direct expression of a polynucleotide in a specific cell type, under certain environmental conditions, and/or during a specific state of development. Suitable tissue-specific promoters can include, but are not limited to, liver-specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g. ITGAM, CD43 promoter, CD14 promoter, CD45 promoter, CD68 promoter), urogenital cell specific promoters (e.g., Pbsn, Upk2, Sbp, Fer114), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are discussed elsewhere herein and can be generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In example embodiments, the vector or system thereof can include one or more elements capable of translocating and/or expressing an engineered polynucleotide of the present invention (e.g., an engineered viral (e.g. AAV) capsid polynucleotide) to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the engineered polynucleotides of the present invention (e.g., an engineered viral (e.g., AAV) capsid polynucleotide) can be operably linked, fused to, or otherwise modified to include a polynucleotide that encodes or is a selectable marker or tag, which can be a polynucleotide or polypeptide. In example embodiments, the polypeptide encoding a polypeptide selectable marker can be incorporated in the engineered polynucleotide of the present invention (e.g., an engineered viral (e.g., AAV) capsid polynucleotide) such that the selectable marker polypeptide, when translated, is inserted between two amino acids between the N- and C-terminus of an engineered polypeptide (e.g., the engineered AAV capsid polypeptide) or at the N- and/or C-terminus of the engineered polypeptide (e.g., an engineered AAV capsid polypeptide). In example embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered AAV capsid system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly (His) tag; solubilization tags such as thioredoxin (TRX) and poly (NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered AAV capsid system or other compositions and/or systems described herein via suitable linker, such as a glycine or glycine serine linkers as short as GS or GG up to (GGGGG) 3 (SEQ ID NO: 4) or (GGGGS) 3 (SEQ ID NO: 5). Other suitable linkers are described elsewhere herein.

The vector or vector system can include one or more polynucleotides encoding one or more targeting moieties. In example embodiments, the targeting moiety encoding polynucleotides can be included in the vector or vector system, such as a viral vector system, such that they are expressed within and/or on the virus particle(s) produced such that the virus particles can be targeted to selective cells, tissues, organs, etc. In example embodiments, the targeting moiety encoding polynucleotides can be included in the vector or vector system such that the engineered polynucleotide(s) of the present invention (e.g., an engineered viral (e.g., AAV) capsid polynucleotide(s)) and/or products expressed therefrom include the targeting moiety and can be targeted to selective cells, tissues, organs, etc. In example embodiments, such as non-viral carriers, the targeting moiety can be attached to the carrier (e.g., polymer, lipid, inorganic molecule etc.) and can be capable of targeting the carrier and any attached or associated engineered polynucleotide(s) of the present invention, the engineered polypeptides, or other compositions of the present invention described herein, to select cells, tissues, organs, etc. In example embodiments, the select cells are endothelial cells of the CNS vasculature.

### Cell-Free Vector and Polynucleotide Expression

In example embodiments, the polynucleotide(s) encoding a targeting motif of the present invention can be expressed from a vector or suitable polynucleotide in a cell-free in vitro system. In example embodiments, the polynucleotide encoding one or more features of the engineered AAV capsid system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In example embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenolpyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E. coli-based systems). In these systems, transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding a targeting motif of the present invention and/or other polynucleotides described herein can be codon optimized. In example embodiments, polynucleotides of the engineered AAV capsid system described herein can be codon optimized. In example embodiments, one or more polynucleotides contained in a vector (“vector polynucleotides”) described herein that are in addition to an optionally codon optimized polynucleotide encoding an n-mer motif, including, but not limited to, embodiments of the engineered AAV capsid system described herein, can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In example embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1):1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a select cell-type, tissue type, organ type, and/or subject type. In example embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. In example embodiments, the polynucleotide is codon optimized for a specific cell type or types. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In example embodiments, the polynucleotide is codon optimized for a specific tissue type. Such tissue types can include, but are not limited to, muscle tissue, connective tissue, nervous tissue, and epithelial tissue. Such codon optimized sequences are within the ambit of the ordinary skilled artisan in view of the description herein. In example embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In example embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In example embodiments, the vector is a non-viral vector or carrier. In example embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an engineered capsid polynucleotide (e.g., an engineered AAV capsid polynucleotide) or other composition of the present invention described herein and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as an engineered AAV capsid polynucleotide of the present invention.

### Naked Polynucleotides

In example embodiments, one or more engineered AAV capsid polynucleotides or other polynucleotides of the present invention described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered AAV capsid polynucleotides or other polynucleotides of the present invention described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In example embodiments, the naked polynucleotide contains only the engineered AAV capsid polynucleotide(s) or other polynucleotides of the present invention. In example embodiments, the naked polynucleotide can contain other nucleic acids and/or polynucleotides in addition to the engineered AAV capsid polynucleotide(s) or other polynucleotides of the present invention described elsewhere herein. The naked polynucleotides can include one or more elements of a transposon system. Transposons and system thereof are described in greater detail elsewhere herein.

### Non-Viral Polynucleotide Vectors

In example embodiments, one or more of the engineered AAV capsid polynucleotides or other polynucleotides of the present invention can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In example embodiments, the non-viral polynucleotide vector can have a conditional origin of replication. In example embodiments, the non-viral polynucleotide vector can be an ORT plasmid. In example embodiments, the non-viral polynucleotide vector can have a minimalistic immunologically defined gene expression. In example embodiments, the non-viral polynucleotide vector can have one or more post-segregationally killing system genes. In example embodiments, the non-viral polynucleotide vector is AR-free. In example embodiments, the non-viral polynucleotide vector is a minivector. In example embodiments, the non-viral polynucleotide vector includes a nuclear localization signal. In example embodiments, the non-viral polynucleotide vector can include one or more CpG motifs. In example embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more engineered AAV capsid polynucleotides or other polynucleotides or molecules of the present invention) included in the non-viral polynucleotide vector. In example embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In example embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In example embodiments, the non-viral polynucleotide vector can be a retrotransposon vector. In example embodiments, the retrotransposon vector includes long terminal repeats. In example embodiments, the retrotransposon vector does not include long terminal repeats. In example embodiments, the non-viral polynucleotide vector can be a DNA transposon vector. DNA transposon vectors can include a polynucleotide sequence encoding a transposase. In example embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In example embodiments, the non-autonomous transposon vectors lack one or more Ac elements.

In example embodiments, a non-viral polynucleotide transposon vector system can include a first polynucleotide vector that contains the engineered AAV capsid polynucleotide(s) or other polynucleotides, or molecules of the present invention described herein flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell, the transposase can be expressed from the second vector and can transpose the material between the TIRs on the first vector (e.g., the engineered AAV capsid polynucleotide(s) or other polynucleotides or molecules of the present invention) and integrate it into one or more positions in the host cell's genome. In example embodiments, the transposon vector or system thereof can be configured as a gene trap.

In example embodiments, the TIRs can be configured to flank a strong splice acceptor site followed by a reporter and/or other gene (e.g., one or more of the engineered AAV capsid polynucleotide(s) or other polynucleotides or molecules of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Chemical Carriers

In example embodiments the engineered AAV capsid polynucleotide(s) or other polynucleotides or other molecules of the present invention described herein can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the engineered AAV capsid polynucleotide(s) of the present invention), (2) those capable of targeting specific or select cells, (3) those capable of increasing delivery of the polynucleotide or other molecules (such as the engineered AAV capsid polynucleotide(s)) of the present invention to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the compositions (including particles, polypeptides, polynucleotides, and other compositions described herein) present invention described herein. Suitable sizes include macro-, micro-, and nano-sized particles.

In example embodiments, the non-viral carrier can be an inorganic particle. In example embodiments, the inorganic particle, can be a nanoparticle. The inorganic particles can be configured and optimized by varying size, shape, and/or porosity. In example embodiments, the inorganic particles are optimized to escape from the reticulo-endothelial system. In example embodiments, the inorganic particles can be optimized to protect an entrapped molecule from degradation. The suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., supermagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In example embodiments, the non-viral carrier can be lipid-based. Suitable lipid-based carriers are also described in greater detail herein. In example embodiments, the lipid-based carrier includes a cationic lipid or an amphiphilic lipid that is capable of binding or otherwise interacting with a negative charge on the polynucleotide to be delivered (e.g., such as an engineered AAV capsid polynucleotide of the present invention). In example embodiments, chemical non-viral carrier systems can include a polynucleotide (such as the engineered AAV capsid polynucleotide(s)) or other composition or molecule of the present invention) and a lipid (such as a cationic lipid). These are also referred to in the art as lipoplexes. Other embodiments of lipoplexes are described elsewhere herein. In example embodiments, the non-viral lipid-based carrier can be a lipid nanoemulsion. Lipid nanoemulsions can be formed by the dispersion of an immiscible liquid in another stabilized emulsifying agent and can have particles of about 200 nm that are composed of the lipid, water, and surfactant that can contain the polynucleotide to be delivered (e.g., the engineered AAV capsid polynucleotide(s) of the present invention). In example embodiments, the lipid-based non-viral carrier can be a solid lipid particle or nanoparticle.

In example embodiments, the non-viral carrier can be peptide-based. In example embodiments, the peptide-based non-viral carrier can include one or more cationic amino acids. In example embodiments, 35 to 40, 45, 50, 55, 60, 65, 70, 75, 80, 85, 90, 95, 99 or 100% of the amino acids are cationic. In example embodiments, peptide carriers can be used in conjunction with other types of carriers (e.g., polymer-based carriers and lipid-based carriers to functionalize these carriers). In example embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the engineered AAV capsid polynucleotides of the present invention), polymethacrylate, and combinations thereof.

In example embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In example embodiments, the non-viral carrier can be a particle that is configured includes one or more of the engineered AAV capsid polynucleotides or other compositions of the present invention describe herein and an environmental triggering agent response element, and optionally a triggering agent. In example embodiments, the particle can include a polymer that can be selected from the group of polymethacrylates and polyacrylates. In example embodiments, the non-viral particle can include one or more embodiments of the compositions microparticles described in US Pat. Pubs. 20150232883 and 20050123596, whose techniques and compositions can be adapted for use in the present invention.

In example embodiments, the non-viral carrier can be a polymer-based carrier. In example embodiments, the polymer is cationic or is predominantly cationic such that it can interact in a charge-dependent manner with the negatively charged polynucleotide to be delivered (such as the engineered AAV capsid polynucleotide(s) of the present invention). Polymer-based systems are described in greater detail elsewhere herein.

### Viral Vectors

In example embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered AAV capsid polynucleotide, cargo, or other composition or molecule of the present invention, into a virus particle and producing said virus particle when used alone or with one or more other viral vectors (such as in a viral vector system). Viral vectors and systems thereof can be used for producing viral particles for delivery of and/or expression and/or generation of one or more compositions of the present invention described herein (including, but not limited to, any viral particle and associated cargo). The viral vector can be part of a viral vector system involving multiple vectors. In example embodiments, systems incorporating multiple viral vectors can increase the safety of these systems. Suitable viral vectors can include adenoviral-based vectors, adeno associated vectors, helper-dependent adenoviral (HdAd) vectors, hybrid adenoviral vectors, and the like. Other embodiments of viral vectors and viral particles produce therefrom are described elsewhere herein. In example embodiments, the viral vectors are configured to produce replication incompetent viral particles for improved safety of these systems.

### Adenoviral Vectors, Helper-Dependent Adenoviral Vectors, and Hybrid Adenoviral Vectors

In example embodiments, the vector can be an adenoviral vector. In example embodiments, the adenoviral vector can include elements such that the virus particle produced using the vector or system thereof can be serotype 2, 5, or 9. In example embodiments, the polynucleotide to be delivered via the adenoviral particle can be up to about 8 kb. Thus, in example embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261. The engineered AAV capsids can be included in an adenoviral vector to produce adenoviral particles containing said engineered AAV capsids.

In example embodiments, the vector can be a helper-dependent adenoviral vector or system thereof. These are also referred to in the field as “gutless” or “gutted” vectors and are a modified generation of adenoviral vectors (see e.g., Thrasher et al. 2006. Nature. 443: E5-7). In embodiments of the helper-dependent adenoviral vector system, one vector (the helper) can contain all the viral genes required for replication but contains a conditional gene defect in the packaging domain. The second vector of the system can contain only the ends of the viral genome, one or more engineered AAV capsid polynucleotides, and the native packaging recognition signal, which can allow selective packaged release from the cells (see e.g., Cideciyan et al. 2009. N Engl J Med. 361:725-727). Helper-dependent Adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered AAV capsid polynucleotides described herein. In example embodiments, the polynucleotide to be delivered via the viral particle produced from a helper-dependent adenoviral vector or system thereof can be up to about 38 kb. Thus, in example embodiments, an adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In example embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In example embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5):2964-2971; Zhang et al. 2013. PloS One. 8(10) e76771; and Cooney et al. 2015. Mol. Ther. 23(4):667-674), whose techniques and vectors described therein can be modified and adapted for use in the engineered AAV capsid system of the present invention. In example embodiments, a hybrid-adenoviral vector can include one or more features of a retrovirus and/or an adeno-associated virus. In example embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered AAV capsid system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered AAV capsid system of the present invention.

### Adeno-Associated Vectors

In an embodiment, the engineered vector or system thereof can be an adeno-associated vector (AAV). See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In example embodiments, the AAV can integrate into a specific or preferred site on chromosome 19 of a human cell with no observable side effects. In example embodiments, the capacity of the AAV vector, system thereof, and/or AAV particles can be up to about 4.7 kb. The AAV vector or system thereof can include one or more engineered capsid polynucleotides described herein.

The AAV vector or system thereof can include one or more regulatory molecules. In example embodiments the regulatory molecules can be promoters, enhancers, repressors and the like, which are described in greater detail elsewhere herein. In example embodiments, the AAV vector or system thereof can include one or more polynucleotides that can encode one or more regulatory proteins. In example embodiments, the one or more regulatory proteins can be selected from Rep78, Rep68, Rep52, Rep40, variants thereof, and combinations thereof. In example embodiments, the promoter can be a tissue specific promoter as previously discussed. In example embodiments, the tissue specific promoter can drive expression of an engineered capsid AAV capsid polynucleotide described herein.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue, and/or organ-selective tropism.

In example embodiments, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In example embodiments, a producing host cell line expresses one or more of the adenovirus helper factors.

The AAV vector or system thereof can be configured to produce AAV particles having a specific serotype. In example embodiments, the serotype can be AAV-1, AAV-2, AAV-3, AAV-4, AAV-5, AAV-6, AAV-8, AAV-9 or any combinations thereof. In example embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in example embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In example embodiments, an AAV vector or system thereof capable of producing AAV particles capable of targeting cardiac tissue can be configured to generate an AAV particle having an AAV-4 serotype. In example embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ selectivity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-selective tropism, which can be the same or different as that of the reference wild-type AAV serotype. In example embodiments, the cell, tissue, and/or selectivity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the bias for the brain can be reduced or eliminated and/or the CNS endothelial cell-septicity increased such that the brain selectivity appears reduced in comparison, thus enhancing the selectivity for the endothelial cells of the CNS vasculature as compared to the wild-type AAV-9.

In example embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same selectivity issues as with the non-hybrid wild-type serotypes previously discussed.

Advantages achieved by the wild-type based hybrid AAV systems can be combined with the increased and customizable cell-selectivity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

A tabulation of certain wild-type AAV serotypes as to these cells can be found in Grimm, D. et al, J. Virol. 82:5887-5911 (2008) reproduced below as Table A. Further tropism details can be found in Srivastava. 2017. Curr. Opin. Virol. 21:75-80 as previously discussed.

TABLE A

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In one example embodiment, the AAV vector or system thereof is AAV rh.74 or AAV rh.10.

In another example embodiment, the AAV vector or system thereof is configured as a “gutless” vector, similar to that described in connection with a retroviral vector. In example embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

### Vector Construction

The vectors described herein can be constructed using any suitable process or technique. In example embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In example embodiments, the vector can have one or more insertion sites, such as a restriction endonuclease recognition sequence (also referred to as a “cloning site”). In example embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of an engineered AAV capsid system described herein are as used in the foregoing documents, such as WO 2014/093622 (PCT/US2013/074667) and are discussed in greater detail herein.

Virus Particle Production from Viral Vectors

### AAV Particle Production

There are two main strategies for producing AAV particles from AAV vectors and systems thereof, such as those described herein, which depend on how the adenovirus helper factors are provided (helper v. helper free). In example embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered AAV capsid polynucleotide(s)). In example embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest between 2 ITRs; (2) a vector that carries the AAV Rep-Cap encoding polynucleotides (e.g., the engineered AAV capsid polynucleotide(s)); and (3) helper polynucleotides. One of skill in the art will appreciate various methods and variations thereof that are both helper and helper free and as well as the different advantages of each system.

The engineered AAV vectors and systems thereof described herein can be produced by any of these methods.

A vector (including non-viral carriers) described herein can be introduced into host cells to thereby produce transcripts, proteins, or peptides, including fusion proteins or peptides encoded by nucleic acids as described herein (e.g., engineered AAV capsid system transcripts, proteins, enzymes, mutant forms thereof, fusion proteins thereof, etc.), and virus particles (such as from viral vectors and systems thereof).

One or more engineered AAV capsid polynucleotides can be delivered using adeno associated virus (AAV), adenovirus or other plasmid or viral vector types as previously described, in particular, using formulations and doses from, for example, U.S. Pat. Nos. 8,454,972 (formulations, doses for adenovirus), 8,404,658 (formulations, doses for AAV) and 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In example embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of engineered AAV capsid system components (e.g., polynucleotides encoding engineered AAV capsid and/or capsid proteins) to cells via particles. The term “particle” as used herein refers to any suitable sized particles for delivery of the engineered AAV capsid system components described herein. Suitable sizes include macro-, micro-, and nano-sized particles. In example embodiments, any of the of the engineered AAV capsid system components (e.g., polypeptides, polynucleotides, vectors and combinations thereof described herein) can be attached to, coupled to, integrated with, otherwise associated with one or more particles or component thereof as described herein. The particles described herein can then be administered to a cell or organism by an appropriate route and/or technique. In example embodiments, particle delivery can be selected and be advantageous for delivery of the polynucleotide or vector components. It will be appreciated that in embodiments, particle delivery can also be advantageous for other engineered capsid system molecules and formulations described elsewhere herein.

Engineered Cells and Organisms Expressing said engineered AAV capsids

Described herein are engineered cells that can include one or more of the engineered AAV capsid polynucleotides, polypeptides, vectors, and/or vector systems. In example embodiments, one or more of the engineered AAV capsid polynucleotides can be expressed in the engineered cells. In example embodiments, the engineered cells can be capable of producing engineered AAV capsid proteins and/or engineered AAV capsid particles that are described elsewhere herein. Also described herein are modified or engineered organisms that can include one or more engineered cells described herein. The engineered cells can be engineered to express a cargo molecule (e.g., a cargo polynucleotide) dependently or independently of an engineered AAV capsid polynucleotide as described elsewhere herein.

A wide variety of animals, plants, algae, fungi, yeast, etc. and animal, plant, algae, fungus, yeast cell or tissue systems may be engineered to express one or more nucleic acid constructs of the engineered AAV capsid system described herein using various transformation methods mentioned elsewhere herein. This can produce organisms that can produce engineered AAV capsid particles, such as for production purposes, engineered AAV capsid design and/or generation, and/or model organisms. In example embodiments, the polynucleotide(s) encoding one or more components of the engineered AAV capsid system described herein can be stably or transiently incorporated into one or more cells of a plant, animal, algae, fungus, and/or yeast or tissue system. In example embodiments, one or more of engineered AAV capsid system polynucleotides are genomically incorporated into one or more cells of a plant, animal, algae, fungus, and/or yeast or tissue system. Further embodiments of the modified organisms and systems are described elsewhere herein. In example embodiments, one or more components of the engineered AAV capsid system described herein are expressed in one or more cells of the plant, animal, algae, fungus, yeast, or tissue systems.

### Engineered Cells

Described herein are various embodiments of engineered cells that can include one or more of the engineered AAV capsid system polynucleotides, polypeptides, vectors, and/or vector systems described elsewhere herein. In example embodiments, the cells can express one or more of the engineered AAV capsid polynucleotides and can produce one or more engineered AAV capsid particles, which are described in greater detail herein. Such cells are also referred to herein as “producer cells”. It will be appreciated that these engineered cells are different from “modified cells” described elsewhere herein in that the modified cells are not necessarily producer cells (i.e., they do not make engineered GTA delivery particles) unless they include one or more of the engineered AAV capsid polynucleotides, engineered AAV capsid vectors or other vectors described herein that render the cells capable of producing an engineered AAV capsid particle. Modified cells can be recipient cells of an engineered AAV capsid particles and can, in example embodiments, be modified by the engineered AAV capsid particle(s) and/or a cargo polynucleotide delivered to the recipient cell. Modified cells are discussed in greater detail elsewhere herein. The term modification can be used in connection with modification of a cell that is not dependent on being a recipient cell. For example, isolated cells can be modified prior to receiving an engineered AAV capsid molecule.

In an embodiment, the invention provides a non-human eukaryotic organism; for example, a multicellular eukaryotic organism, including a eukaryotic host cell containing one or more components of an engineered delivery system described herein according to any of the described embodiments. In other embodiments, the invention provides a eukaryotic organism; preferably a multicellular eukaryotic organism, comprising a eukaryotic host cell containing one or more components of an engineered delivery system described herein according to any of the described embodiments. In example embodiments, the organism is a host of AAV.

In particular embodiments, the plants, algae, fungi, yeast, etc., cells or parts obtained are transgenic plants, comprising an exogenous DNA sequence incorporated into the genome of all or part of the cells.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21 (DE3), DL21 (DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue (DE3), BLR, C41 (DE3), C43 (DE3), Lemo21 (DE3), Shuffle T7, ArcticExpress and ArticExpress (DE3).

The engineered cell can be a eukaryotic cell. The eukaryotic cells may be those of or derived from a particular organism, such as a plant or a mammal, including, but not limited to, human, or non-human eukaryote or animal or mammal as herein discussed, e.g., mouse, rat, rabbit, dog, livestock, or non-human mammal or primate. In example embodiments the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In example embodiments, the engineered cell can be a fungal cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In example embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In example embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In example embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella 60sabelline).

In example embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In example embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In example embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In example embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific or selective regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In example embodiments, the engineered cell is a cell obtained from a subject. In example embodiments, the subject is a healthy or non-diseased subject. In example embodiments, the subject is a subject with a desired physiological and/or biological characteristic such that when an engineered AAV capsid particle is produced it can package one or more cargo polynucleotides that can be related to the desired physiological and/or biological characteristic and/or capable of modifying the desired physiological and/or biological characteristic. Thus, the cargo polynucleotides of the produced engineered AAV capsid particle can be capable of transferring the desired characteristic to a recipient cell. In example embodiments, the cargo polynucleotides are capable of modifying a polynucleotide of the engineered cell such that the engineered cell has a desired physiological and/or biological characteristic.

In example embodiments, a cell transfected with one or more vectors described herein is used to establish a new cell line comprising one or more vector-derived sequences.

The engineered cells can be used to produce engineered viral (e.g., AAV) capsid polynucleotides, vectors, and/or particles. In example embodiments, the engineered viral (e.g., AAV) capsid polynucleotides, vectors, and/or particles are produced, harvested, and/or delivered to a subject in need thereof. In example embodiments, the engineered cells are delivered to a subject. Other uses for the engineered cells are described elsewhere herein. In example embodiments, the engineered cells can be included in formulations and/or kits described elsewhere herein.

The engineered cells can be stored short-term or long-term for use at a later time. Suitable storage methods are generally known in the art. Further, methods of restoring the stored cells for use (such as thawing, reconstitution, and otherwise stimulating metabolism in the engineered cell after storage) at a later time are also generally known in the art.

### Formulations

The compositions, polynucleotides, polypeptides, particles, cells, vector systems and combinations thereof described herein can be contained in a formulation, such as a pharmaceutical formulation. In example embodiments, the formulations can be used to generate polypeptides and other particles that include one or more selective targeting moieties described herein. In example embodiments, the formulations can be delivered to a subject in need thereof. In example embodiments, component(s) of the engineered AAV capsid system, engineered cells, engineered AAV capsid particles, and/or combinations thereof described herein can be included in a formulation that can be delivered to a subject or a cell. In example embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In example embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In example embodiments, the amount of the one or more of the polypeptides, polynucleotides, vectors, cells, virus particles, nanoparticles, other delivery particles, and combinations thereof described herein contained in the pharmaceutical formulation can range from about 1 pg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 pg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

In embodiments, were engineered AAV capsid particles are included in the formulation, the formulation can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In example embodiments, the formulation can be 0.1 to 100 mL in volume and can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered AAV capsid particles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoirenti-histamines, anti-infectives, chemotherapeutics, and combinations thereof.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-a, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammator (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), mebicar, fabomotizole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammatoires (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammatoires (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives)

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, parconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, boceprevir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, cefazoline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, Fosfomycin, metronidazole, aztreonam, bacitracin, penicillin (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, Fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In example embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In example embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In example embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In example embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In example embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for nasal or inhalation administration include aerosols, solutions, suspension drops, gels, or dry powders. In example embodiments, the one or more of the polypeptides, polynucleotides, vectors, cells, and combinations thereof described herein is contained in a dosage form adapted for inhalation is in a particle-size-reduced form that is obtained or obtainable by micronization. In example embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In example embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In example embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including, but not limited to, sealed ampoules or vials. The doses can be lyophilized and resuspended in a sterile carrier to reconstitute the dose prior to administration. Extemporaneous injection solutions and suspensions can be prepared in example embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In example embodiments, the predetermined amount of the Such unit doses may therefore be administered once or more than once a day. Such pharmaceutical formulations may be prepared by any of the methods well known in the art.

### Kits

Also described herein are kits that contain one or more of the one or more of the compositions, polypeptides, polynucleotides, vectors, cells, or other components described herein and combinations thereof and pharmaceutical formulations described herein. In embodiments, one or more of the polypeptides, polynucleotides, vectors, cells, and combinations thereof described herein can be presented as a combination kit. As used herein, the terms “combination kit” or “kit of parts” refers to the compounds, or formulations and additional components that are used to package, screen, test, sell, market, deliver, and/or administer the combination of elements or a single element, such as the active ingredient, contained therein. Such additional components include, but are not limited to, packaging, syringes, blister packages, bottles, and the like. The combination kit can contain one or more of the components (e.g., one or more of the one or more of the polypeptides, polynucleotides, vectors, cells, and combinations thereof) or formulation thereof can be provided in a single formulation (e.g., a liquid, lyophilized powder, etc.), or in separate formulations. The separate components or formulations can be contained in a single package or in separate packages within the kit. The kit can also include instructions in a tangible medium of expression that can contain information and/or directions regarding the content of the components and/or formulations contained therein, safety information regarding the content of the components(s) and/or formulation(s) contained therein, information regarding the amounts, dosages, indications for use, screening methods, component design recommendations and/or information, recommended treatment regimen(s) for the components(s) and/or formulations contained therein. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g., a web interface.

In one embodiment, the invention provides a kit comprising one or more of the components described herein. In example embodiments, the kit comprises a vector system and instructions for using the kit. In example embodiments, the vector system includes a regulatory element operably linked to one or more engineered polynucleotides, such as those containing a selective targeting moiety, as described elsewhere herein and, optionally, a cargo molecule, which can optionally be operably linked to a regulatory element. The one or more engineered polynucleotides such as those containing a selective targeting moiety, as described elsewhere herein and, can be included on the same or different vectors as the cargo molecule in embodiments containing a cargo molecule within the kit.

Further embodiments are illustrated in the following Examples which are given for illustrative purposes only and are not intended to limit the scope of the invention.

### EXAMPLES

### Example 1

Here Applicants tested whether BI30-mediated delivery of the gene for a lysosomal enzyme to endothelial cells would provide cross-correction to neuronal cells in an LSD mouse model. Unlike previous related studies, endothelial targeting of AAV-BI30 does not depend on disease state, transduces efficiently across the arterio-venous axis, and has potential transferability to humans (Chen, 2009; Dogbevia, 2020). Moreover, widespread transduction of endothelial cells could rescue enzyme activity to greater extent than direct intracerebral or intrathecal administration. Other AAV capsids with similar endothelial cell-selective tropisms (PCT/US2022/073968) could similarly be used as vehicles for delivery of the IDS gene or other genes as therapies for MPS II or other LSDs that can leverage cross correction.

Here Applicants deliver the human gene for iduronate 2-sulfatase (IDS) to endothelial cells with BI30 to cross-correct both peripheral and CNS defects in a mouse model for Mucopolysaccharidosis type II (MPS II, Hunter syndrome). MPS II is an LSD caused by pathogenic mutations in the IDS gene which lead to the dysfunction of the IDS enzyme and the build-up of sugar molecules called glycosaminoglycans (GAGs) that the functional enzyme is responsible for metabolizing. This build-up of sugars affects many tissues throughout the body, including the brain, generally causing coarsened facial features, enlarged organs, decline in intellectual function, and a shortened life span to 10-20 years (Scarpa, 2007). ERT and haematopoietic stem cell transplantation (HSCT) for MPSII does not correct CNS symptoms. Intravenous delivery of AAV gene therapy also does not normalize GAG levels in the brain, though direct intracisternal or intracerebroventricular injection of AAV9 shows some amelioration of CNS symptoms in a MPSII mouse model (Wood, 2022). In this proposal, Applicants use the MPSII mouse model which lacks IDS gene expression, and is characterized by progressive GAG buildup, skeletal and neurological disease, and premature death by 70 weeks of age (Muenzer, 2002). For a proof-of-concept study, Applicants investigated the cross-correction effects of endothelial expression of IDS with the BI30-IDS vector at early ages before overt disease onset to observe rescue of IDS activity and normalization of GAG buildup in the brain in comparison to direct neuronal transduction.

### Methods

### In-Vitro Expression of IDS

Human embryonic kidney HEK293T cells (ATCC #3579061) were STR authenticated and verified mycoplasma-free. Cells were cultured at a seeding density of 50,000 cells per well in a 24-well plate in DMEM with GlutaMAX (Gibco, 10569010) supplemented with 5% FBS and 1× non-essential amino acid solution (NEAA) (Gibco, 11140050) and grown at 37° C. on Poly-L-lysine and laminin coated coverslips. For AAV transduction, media was removed from the cells, cells were counted, and the appropriate volume of AAV to achieve the target MOI was diluted in 500 uL DMEM and added directly to the cells. For transient transfection, cells were transiently transfected with pAAV-CAG-hIDS-HA-WPRE (1.5 ug) using Fugene6 (Promega) according to manufacturer protocol. Cells were fixed and immunostained 48 hours after transduction or transfection.

HT-1080 cells (ATCC #CCL-121) were cultured in Eagle's Minimum Essential Medium (ATCC #30-2003) supplemented with 10% fetal bovine serum. Cells were seeded and transduced with AAV as described above.

### Immunostaining

Cells were fixed with 4% paraformaldehyde for 30 min at RT and then blocked with 10% normal goat serum (NGS) for 15 min at RT. Cells were then incubated in 1% NGS containing 1:100 anti-IDS (Boster, EK1452-DA) overnight at 4C. Cells were then washed 3× with PBS and incubated at RT in 1% NGS containing 1:500 Alexa Fluor 594-conjugated anti-rabbit antibody (A-11012; Molecular Probes) for 3 hours in the dark. Cells were then washed 3× in PBS, mounted with Fluoromount-G (SouthernBiotech) on glass slides, and imaged on a (type) inverted confocal microscope at ×20 magnification.

### ELISA

ELISA assays for human IDS were carried out with the Human IDS/Iduronate 2 Sulfatase ELISA Kit according to manufacturer's guidelines (Boster, EK1452). Cell media was collected, centrifuged to remove cell debris, and stored at-80C. Cell lysate was harvested by placing cells on ice and washing 3× with ice cold PBS, followed by the addition of 50 μL of extraction buffer (100 mM Tris pH 7.4, 150 mM NaCl, 2 mM EDTA, 11% Triton X-100, 0.50% Sodium deoxycholate). Cells were collected, vortexed, incubated on ice for 15 minutes, centrifuged for 10 minutes at 10,000×g in 4C, and stored at-80C. Mouse serum was collected as described below. Human IDS standard was reconstituted in sample diluent to a concentration of 50 ng/mL. Serial dilutions of standard and samples were added to sample plate for 90 minutes at 37C. Samples were removed, biotinylated anti-hIDS antibody (1:100) was added to each well, and the plate was incubated for 1 hour at 37C. Plates were then emptied, washed, and avidin-biotin-peroxidase was added to each well for 30 minutes at 37C. Plates were then emptied, washed, and TMB substrate was assess to each well for 15 minutes at 37C. Reactions were stopped and ELISA plates were read on a microplate reader with absorbance O.D. at 450 nm.

### AAV Vector Production

Recombinant AAVs were generated following a previously published protocol with minor modifications as described below. HEK293T/17 cells (ATCC, CRL-11268) were seeded at 22 million cells per 15 cm plate the day before transfection and grown in DMEM with GlutaMAX (Gibco, 10569010) supplemented with 5% FBS and 1× non-essential amino acid solution (NEAA) (Gibco, 11140050). The next day, the cells were triple transfected with 39.93 ug of total plasmid DNA encoding Rep-Cap, pHelper, and an ITR-flanked transgene at a plasmid ratio of 4:2:1, respectively, using polyethyleimine (PEI) MAX (Polyscience, 24765-1) at a DNA: PEI ratio of 1:3.5. Twenty hours post-transfection, the media was changed to fresh DMEM with GlutaMAX supplemented with 5% FBS and 1×NEAA. Seventy-two hours post-transfection cells were scraped and pelleted at 2000 RCF×10 minutes. The pellets were resuspended in 7 mL of Salt Active Nuclease (SAN) digestion buffer (500 mM NaCl, 40 mM Tris-base, 10 mM MgCl2, SAN enzyme (ArcticZymes, #70920-202) at 100 U/mL) for every 10 plates and incubated at 37° C. for 1.5 hours. Afterwards, the lysate was clarified at 2000 RCF×10 min and loaded onto a density step gradient containing OptiPrep (Cosmo Bio, AXS-1114542) at 60%, 40%, 25%, and 15% at a volume of 5, 5, 6, and 6 mL respectively in OptiSeal tubes (Beckman, 361625). The step gradients were spun in a Beckman Type 70ti rotor (Beckman, 337922) in a Sorvall WX+ultracentrifuge (Thermo Scientific, 75000090) at 69,000 RPM for 1 hour at 18° C. Afterwards, 4-4.5 mL of the 40-60% interface was extracted using a 16-gauge needle, filtered through a 0.22 μm PES filter, and then buffer exchanged with 100K MWCO protein concentrators (Thermo Scientific, 88532) into PBS containing 0.001% Pluronic F-68 and concentrated down to a volume of 500 μL. The concentrated virus was then filtered through a 0.22 μm PES filter and stored at 4° C. or −80° C.

### Virus Titering

Purified virus was incubated with 1000 U/mL Turbonuclease (Sigma T4330-50KU) with 1× DNase I reaction buffer (NEB B0303S) at 37° C. for one hour. The endonuclease solution was inactivated with 0.5M, pH 8.0 EDTA at room temperature for 5 minutes and then at 70° C. for 10 minutes. AAV genomes were released by incubation with 100 ug/mL Proteinase K (Qiagen, 19131) in 1M NaCl, 1% N-lauroylsarcosine, and in UltraPure DNase/RNase-Free water at 56° C. for 2 to 16 hours before heat inactivation at 95° C. for 10 minutes. The nuclease-resistant AAV genomes were diluted between 460-460,000× and 2 μL of the diluted samples were used as input in a ddPCR supermix for probes (Bio-Rad, 1863023) with 900 nM ITR2 Forward (5′-GGAACCCCTAGTGATGGAGTT-3′ (SEQ ID NO: 1106)), 900 nM ITR2_Reverse (5′-CGGCCTCAGTGAGCGA-3′ (SEQ ID NO: 1107)), and 250 nM ITR2_Probe (5′-HEX-CACTCCCTC-ZEN-TCTGCGCGCTCG-IABKFQ-3′ (SEQ ID NO: 1108)). The ITR2 Probe contained the following modifications-5′ HEX dye, ZEN internal quencher, and 3′ Iowa Black fluorescent quencher (IDT, PrimeTime qPCR Probes). Droplets were generated using a QX100 Droplet Generator, transferred to thermocycler, and cycled according to the manufacturer's protocol with an annealing/extension of 58° C. for 1 minute. Finally, droplets were read on a QX100 Droplet Digital System to determine titers.

### Animals

IDS-KO (B6N.Cg-Idstm1Muen/J) mice were obtained from the Jackson Laboratories. The IDS phenotype was confirmed by PCR of DNA isolated from ear clippings using the primers 5′-TGATGTCTTATAGAGATAAGCATTAGGGTCTT-3′ (SEQ ID NO: 1109) (forward) and 5′-TAATCTCATAAAAAAGCACTCATTGTG-3′ (SEQ ID NO: 1110) (reverse), and the reporter sequence 5′-CCCATCAAAGCAATCAC-3′ (SEQ ID NO: 1111). Animals were fed ad libitum with standard chow diet and maintained on a 12-hr light/dark cycle. Their weight was monitored weekly for the duration of the study.

### Vector Administration and Sample Collection

Four to seven-week-old mice received intra-venous injections via the retro-orbital route of 3×10¹¹ vg/animal of AAV9, AAV-BI30 or AAV-PHP.eB vectors expressing human IDS (with or without the signal sequence). Retro-orbital injections were performed under anesthesia with 3% isoflurane and 97% oxygen. WT C57BL/6J mice served as healthy controls. Four weeks after vector administration, mice were anesthetized using 3% isoflurane. Urine was collected from the bladder using a 30G needle. Blood was extracted by cardiac puncture into a tube containing a serum-separating gel (BD). Mice were then transcardially perfused with 30 mL of phosphate buffered saline (PBS). The tissues of interest were collected and snap-frozen. Blood was allowed to clot for 30 min at room temperature. Then, it was spun at 12,000×g for 2 min. The serum was transferred to a new tube and stored at −80° C.

### Open-Field Test

One day before sacrifice, mice were subjected to an open-field test. Animals were allowed to acclimate in the behavioral testing room for 1 hour. Mice were then placed inside a brightly lit chamber crossed by photobeams that detect horizontal and vertical movements. Exploratory and motor activities were recorded for 60 minutes. All animals were otherwise naïve to the test.

### Tissue Preparation

Tissues of interest were weighed after being thawed on ice. The tissue was then homogenized in 2× volume of ice-cold water+complete proteinase inhibitor (Roche), using the GenoGrinder (SPEX Sample Prep). Tubes were spun at 10,000×g and 4° C. for 2 min. Homogenate was transferred to a new, pre-chilled tube and incubated on ice for 15-30 min. Next, samples were centrifuged at 12,000×g and 4° C. for 20 min and the supernatant was collected. Protein concentration in the tissue lysate was determined using the Pierce BCA protein assay (Thermo Scientific). The lysate was stored at −80° C. until further processing. Urine samples were centrifuged at 12,000×g and 4° C. for 20 min. The supernatant was collected for the assays.

### Total GAG Quantification

Total GAG Quantification was performed according to the manufacturer's instructions. In brief, 0-10 uL of GAG standard (1 mg/mL) were added per well, to prepare the standard curve. For urine, 15 μL of sample were added per well. For tissues, serial dilutions of the lysate ranging from 1:2 to 1:8 were prepared in water. 10 μL of tissue lysate or diluted sample were used per well. Volumes were adjusted to 100 uL using GAG Assay Buffer. Then, 200 μL of GAG probe were added per well. The plate was incubated for 2 min at room temperature. Absorbance at 400 nm was measured using the plate reader. GAG content in urine samples was normalized to creatinine concentration. In the case of tissues, GAG content was normalized to wet tissue mass.

### Total IDS Enzyme Activity

Iduronate-2-sulfatase enzyme activity was quantified as described previously. Briefly, a small volume of tissue lysate was diluted in water to achieve a protein concentration of 1.5 mg/mL (15 ug per 10 uL). Then, two-fold serial dilutions of serum or tissue lysate samples were prepared in water. 40 μL of reaction mix (0.1 M sodium acetate, 10 mM lead acetate, 0.01% Tween-20, 0.3 mM MU-αIdoA-2S, and excess a-iduronidase) and 10 μL of sample were added per well. Plates were incubated at 37° C. for 3 hours. Reactions were terminated by the addition of 200 μL of stop buffer (0.5 M sodium carbonate, 0.025% Triton-X100, pH 10.7). Fluorescence (λex=365 nm; λem=445 nm) was measured on a plate reader.

### Non-Secreting IDS Rationale and Characterization

Applicants are investigating whether IDS secreted from endothelial cells can cross correct to cells not transduced by BI30 (i.e., neurons). To test this, Applicants constructed a variant of the IDS sequence that Applicants hypothesized to not secrete by deleting the N-terminus signal peptide (residues 1-25) and propeptide sequence (residues 26-33).

Wilson P J, Morris C P, Anson D S, Occhiodoro T, Bielicki J, Clements P R, Hopwood J J. Hunter syndrome: isolation of an iduronate-2-sulfatase cDNA clone and analysis of patient DNA. Proc Natl Acad Sci USA. 1990 November; 87(21):8531-5. doi: 10.1073/pnas.87.21.8531. PMID: 2122463; PMCID: PMC54990.

Holmes R S. Comparative studies of vertebrate iduronate 2-sulfatase (IDS) genes and proteins: evolution of A mammalian X-linked gene. 3 Biotech. 2017 May; 7(1):22. doi: 10.1007/s13205-016-0595-3. Epub 2017 Apr. 11. PMID: 28401457; PMCID: PMC5388652.

### In Vitro Characterization

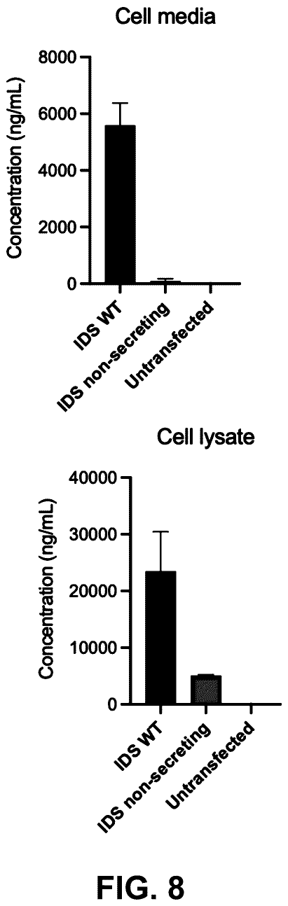

Comparing WT-IDS to ‘no signal sequence’-IDS by transfecting HEK293T cells in vitro and carrying out ELISA against IDS in the cell media and cell lysate (See FIG. 8), Applicants can see that the ‘no signal sequence’-IDS condition is detected less in the cell media, close to untransfected cells, whereas signal is still detectable in the cell lysate. However, signal in cell lysate for ‘no signal sequence’-IDS is not as high was WT-IDS. This could be due to decreased expression or function. This could also be because these cells are not taking up extra IDS that has been secreted into the media.

The ‘no signal sequence’-IDS (BI30: IDS NS) has decreased activity in the serum suggesting a lack of secretion. However, there is also less activity in the liver lysate. Like in the in vitro experiment, this could be due to decreased expression or function in transduced liver cells, or it could be because liver cells in this condition are not taking up extra IDS circulating in the serum. It should be noted that in liver lysate, there is still a significant increase in enzyme activity compared to the WT control, indicating that the construct is still functional.

### Discussion

The limited accessibility of the CNS has so far represented a major obstacle for the delivery of therapeutic enzymes to diseased neurons and glial cells. In this work, Applicants show that systemic administration of AAV-BI30 results in the transduction of endothelial cells in the CNS and some peripheral organs. Gene expression by the brain endothelium was sufficient to correct the pathological GAG accumulation throughout the brain in a mouse model of MPS II. Gene transfer mediated by AAV-BI30 can also significantly enhance IDS enzyme activity in the serum and liver of IDS-KO mice. Both effects are limited when enzyme secretion from transduced cells is halted. The data suggest that gene delivery to the CNS endothelium represents a promising strategy to address neurological damage derived from MPS II. This approach could be readily harnessed for the treatment of a variety of LSDs.

### REFERENCES

Sands M S, Haskins M E. CNS-directed gene therapy for lysosomal storage diseases. Acta Paediatr. 2008 April; 97(457):22-7. doi: 10.1111/j.1651-2227.2008.00660.x. PMID: 18339183; PMCID: PMC3340572.
Sheth J, Nair A. Treatment for Lysosomal Storage Disorders. Curr Pharm Des. 2020; 26(40):5110-5118. doi: 10.2174/1381612826666201015154932. PMID: 33059565.
Brooks D A, Kakavanos R, Hopwood J J. Significance of immune response to enzyme-replacement therapy for patients with a lysosomal storage disorder. Trends Mol Med. 2003 October; 9(10):450-3. doi: 10.1016/j.molmed.2003.08.004. PMID: 14557058.
Vogler C, Levy B, Grubb J H, Galvin N, Tan Y, Kakkis E, Pavloff N, Sly W S. Overcoming the blood-brain barrier with high-dose enzyme replacement therapy in murine mucopolysaccharidosis VII. Proc Natl Acad Sci USA. 2005 Oct. 11; 102(41):14777-82. doi: 10.1073/pnas.0506892102. Epub 2005 Sep. 14. PMID: 16162667; PMCID: PMC1253584.
Sevin C, Deiva K. Clinical Trials for Gene Therapy in Lysosomal Diseases With CNS Involvement. Front Mol Biosci. 2021 Sep. 16; 8:624988. doi: 10.3389/fmolb.2021.624988. PMID: 34604300; PMCID: PMC8481654.
Nagree M S, Scalia S, McKillop W M, Medin J A. An update on gene therapy for lysosomal storage disorders. Expert Opin Biol Ther. 2019 July; 19(7):655-670. doi: 10.1080/14712598.2019.1607837. Epub 2019 May 6. PMID: 31056978.
Li C, Samulski R J. Engineering adeno-associated virus vectors for gene therapy. Nat Rev Genet. 2020 April; 21(4):255-272. doi: 10.1038/s41576-019-0205-4. Epub 2020 Feb. 10. PMID: 32042148.
Golebiowski D, van der Bom IMJ, Kwon C S, Miller A D, Petrosky K, Bradbury A M, Maitland S, Kühn A L, Bishop N, Curran E, Silva N, GuhaSarkar D, Westmoreland S V, Martin D R, Gounis M J, Asaad W F, Sena-Esteves M. Direct Intracranial Injection of AAVrh8 Encoding Monkey β-N-Acetylhexosaminidase Causes Neurotoxicity in the Primate Brain. Hum Gene Ther. 2017 June; 28(6):510-522. doi: 10.1089/hum.2016.109. Epub 2017 Jan. 26. PMID: 28132521; PMCID: PMC5488349.
Johnston S, Parylak S L, Kim S, Mac N, Lim C, Gallina I, Bloyd C, Newberry A, Saavedra C D, Novak O, Gonçalves J T, Gage F H, Shtrahman M. AAV ablates neurogenesis in the adult murine hippocampus. Elife. 2021 Jul. 14; 10: e59291. doi: 10.7554/eLife.59291. PMID: 34259630; PMCID: PMC8331179.
Krolak T, Chan K Y, Kaplan L, Huang Q, Wu J, Zheng Q, Kozareva V, Beddow T, Tobey I G, Pacouret S, Chen A T, Chan Y A, Ryvkin D, Gu C, Deverman B E. A High-Efficiency AAV for Endothelial Cell Transduction Throughout the Central Nervous System. Nat Cardiovasc Res. 2022 April; 1(4):389-400. doi: 10.1038/s44161-022-00046-4. Epub 2022 Apr. 13. PMID: 35571675; PMCID: PMC9103166.
Chen Y H, Chang M, Davidson B L. Molecular signatures of disease brain endothelia provide new sites for CNS-directed enzyme therapy. Nat Med. 2009 October; 15(10):1215-8. doi: 10.1038/nm.2025. Epub 2009 Sep. 13. PMID: 19749771; PMCID: PMC3181494.
Dogbevia G, Grasshoff H, Othman A, Penno A, Schwaninger M. Brain endothelial specific gene therapy improves experimental Sandhoff disease. J Cereb Blood Flow Metab. 2020 June; 40(6):1338-1350. doi: 10.1177/0271678X19865917. Epub 2019 Jul. 29. PMID: 31357902; PMCID: PMC7238384.
Scarpa M. Mucopolysaccharidosis Type II. 2007 Nov. 6 [Updated 2018 Oct. 4]. In: Adam M P, Everman D B, Mirzaa G M, et al., editors. GeneReviews® [Internet]. Seattle (WA): University of Washington, Seattle; 1993-2023. Available from: www.ncbi.nlm.nih.gov/books/NBK1274/.
Wood S R, Bigger B W. Delivering gene therapy for mucopolysaccharide diseases. Front Mol Biosci. 2022 Sep. 12; 9:965089. doi: 10.3389/fmolb.2022.965089. PMID: 36172050; PMCID: PMC9511407.
Muenzer J, Lamsa J C, Garcia A, Dacosta J, Garcia J, Treco D A. Enzyme replacement therapy in mucopolysaccharidosis type II (Hunter syndrome): a preliminary report. Acta Paediatr Suppl. 2002; 91(439):98-9. doi: 10.1111/j.1651-2227.2002.tb03115.x. PMID: 12572850.

### Example 2-AAV BI-30 and Variants Thereof

### 7-Mer Linear Insertion N2KR5 Motif Description

In an example embodiment the composition of the targeting moiety is in AAV9 between positions 588Q (Gln) and 589A (Ala), and is exemplified by X1-X2-X3-X4-X5-X6-X7, where each X represents an amino acid. In an embodiment, Position X1 is selected from the group consisting of amino acids G, M, T, S, N, D, L, H, P, I, V, Q, Y, W, F, A, E. Position X3 is selected from the group consisting of amino acids N, S, T, H, D, A, Y, M, Q, E, R, G, V. Position X4 is selected from the group consisting of T, V, I, A, M, S, H, W, N. Position X5 is selected from R or K. Position X6 is selected from the group consisting of N, S, G, D, P, T, H, Q, A, Y. Position X7 is selected from the group consisting of T, Y, W, N, V, I, H, M, S, G, A, Q, F, D, P, R, L.

In an example embodiment, the composition of X1, X3, X4, X6, and X7 are independently selected from the following groups. Position X1 is selected from the group consisting of G, M, T, S, N, D. Position X3 is selected from the group consisting of N, S, T, H, D. Position X4 is selected from the group consisting of T, V, I, A. Position X6 is selected from the group consisting of N, S, G, D, P. Position X7 is selected from the group consisting of T, Y, W, N, V, I, H, M, S, G, A, Q, F, D, P, R, L.

In an example embodiment, the composition of the n-mer at position X1 is R or K and X3, X4, X6 and X7 are D or E.

In an example embodiment, the composition of the n-mer at position X1 is not R, K or C; X3 is not W, F, K, C, I, P, or L; X4 is not Y, G, P, D, C, Q, R, K, E, F, L, or R; X6 is not R, I, W, V, F, C, L, E, or K; or X7 is not C, K, E.

The overall charge of the 7-mer sequence is between 0 and +2
X1-N-X3-X4-[K/R]-X6-X7
X1-X2-N-X3-[T/V/I/A/M/S/H/W/N]-[K/R]-X6-X7
X1-X2-N-X3-[T/V/I/A]-[K/R]-X6-X7
X1-X2-N-X3-[T/V/I/A]-[K/R]-X6-X7, where the overall charge of the 7-mer at neutral pH is between 0 and +2.
[G/M/T/S/N/D]-N-[N/S/T/H/D]-[T/V/I/A]-[K/R]-[N/S/G/D/P]-[T/Y/W/N/V/I/H/M/S/G/A/Q/F/D/P/R/L], where the overall charge of the 7-mer at neutral pH is between 0 and +2.

Specific examples of validated sequences (BI30: NNSTRGG (SEQ ID NO: 1), BI31: GNSARNI (SEQ ID NO: 2), and BI55: GNSVRDF (SEQ ID NO: 3)).

TABLE 1
N2KR5 motif containing AAV9 7-mer variants enriched through an in vivo brain
biodistribution screen in C57BL/6J mice using CMV-AAV-Express

| SEQ ID | AA | SEQ ID | AA | SEQ ID | AA | SEQ ID | AA |
|---|---|---|---|---|---|---|---|
| NO | sequence | NO | sequence | NO | sequence | NO | sequence |
| 6 | ANAAKNY | 7 | FNGIKEM | 8 | NNHTREV | 9 | TNKIRDP |
| 10 | ANALKNQ | 11 | FNKDKED | 12 | NNMIRAV | 13 | TNNTRQN |
| 14 | ANATRLQ | 15 | FNSTRPD | 16 | NNNTKNI | 17 | TNNTRVG |
| 18 | ANATRYQ | 19 | GNATKAG | 20 | NNNTRNH | 21 | TNNVKSG |
| 22 | ANEYKAH | 23 | GNDVKDI | 24 | NNQTRMM | 25 | TNPDRSA |
| 26 | ANGTKNG | 27 | GNETRYT | 28 | NNSDKVK | 29 | TNQTKVH |
| 30 | ANIVKLQ | 31 | GNFVKSG | 32 | NNSIKSQ | 33 | TNSAKPY |
| 34 | ANLAKVM | 35 | GNHSKIQ | 36 | NNSTRTA | 37 | TNSTKGA |
| 38 | ANLVKTT | 39 | GNIIRVH | 40 | NNTIKNG | 41 | TNSTRIG |
| 42 | ANPNKSV | 43 | GNLPKIA | 44 | NNVVKNI | 45 | TNSVKAH |
| 46 | ANSERFL | 47 | GNNIRTQ | 48 | NNYTKHQ | 49 | TNTLKNV |
| 50 | ANSIRDV | 51 | GNNSKPP | 52 | NNYTRPI | 53 | TNTTRSP |
| 54 | ANSTRTW | 55 | GNNTKMG | 56 | PNALRMV | 57 | VNAIRDY |
| 58 | ANTTKIE | 59 | GNPEKND | 60 | PNEQKVI | 61 | VNATRVY |
| 62 | ANVVKQF | 63 | GNQTKNG | 64 | PNIIKQN | 65 | VNGGKFM |
| 66 | CNEGRSC | 67 | GNQTRGG | 68 | PNLSKER | 69 | VNITKNF |
| 70 | DNDHRDI | 71 | GNQVKGG | 72 | PNMQKQI | 73 | VNITKSW |
| 74 | DNDSKSS | 75 | GNRSKDY | 76 | PNSDKLK | 77 | VNMTKPI |
| 78 | DNDVKGG | 79 | GNSVKNG | 80 | PNVAKQQ | 81 | VNMVRTF |
| 82 | DNGTKTF | 83 | GNTIRQA | 84 | QNALRNI | 85 | VNQVKNH |
| 86 | DNGVRGY | 87 | GNTVKGS | 88 | QNEVRNG | 89 | VNVERKL |
| 90 | DNHIKPS | 91 | GNVVKST | 92 | QNFVKIS | 93 | VNVGKGP |
| 94 | DNIFKEF | 95 | HNANKML | 96 | QNLGKFI | 97 | WNGNRAA |
| 98 | DNKIKST | 99 | HNEPKNH | 100 | QNMDKPA | 101 | YNATRGG |
| 102 | DNKLRPI | 103 | HNLDKTH | 104 | QNMVRGN | 105 | YNSTRNG |
| 106 | DNMIRKV | 107 | HNLNKFT | 108 | QNQTRIQ | 109 | YNTQKEI |
| 110 | DNPSRSV | 111 | HNPIRFP | 112 | QNTIRMY | 113 | YNTTKDK |
| 114 | DNQMKFV | 115 | INKEKFT | 116 | QNTTKAG | | |
| 117 | DNTERIP | 118 | INSDKSL | 119 | QNYTKNN | | |
| 120 | DNTERPA | 121 | KNDLRNE | 122 | RNDTKGG | | |
| 123 | DNTNKEE | 124 | KNEPKNL | 125 | SNAIKLS | | |
| 126 | ENADKGR | 127 | KNEVKNA | 128 | SNAPRIV | | |
| 129 | ENDPKAV | 130 | LNAMRPL | 131 | SNAPRQV | | |
| 132 | ENGLKYP | 133 | LNGERQI | 134 | SNAVKSA | | |
| 135 | ENHVKGQ | 136 | LNKDKLI | 137 | SNDIRNN | | |
| 138 | ENKERDV | 139 | LNLNRLE | 140 | SNDKRPL | | |
| 141 | ENKIKLV | 142 | MNGSKTA | 143 | SNHIKSA | | |
| 144 | ENLERLK | 145 | MNQQKLV | 146 | SNHTKNF | | |
| 147 | ENNVKAV | 148 | MNQTRGG | 149 | SNLQKFI | | |
| 150 | ENPIKST | 151 | MNSTKNG | 152 | SNLQKVE | | |
| 153 | ENQIRSL | 154 | MNSVRFM | 155 | SNQVRAL | | |
| 156 | ENQVKQY | 157 | MNVTKQT | 158 | SNSTKSA | | |
| 159 | ENQVRME | 160 | NNATRAA | 161 | SNSTRAP | | |
| 162 | ENQYKAI | 163 | NNATRPA | 164 | SNTTRPI | | |
| 165 | ENTSRTQ | 166 | NNGTRLS | 167 | SNVNRLV | | |
| 168 | ENVERLK | 169 | NNHDKSK | 170 | SNVTKWV | | |
| 171 | ENVIRSV | 172 | NNHIRET | 173 | TNATKGG | | |
| 174 | FNAERMT | 175 | NNHIRYH | 176 | TNDDKYQ | | |
| 177 | FNDAKGT | 178 | NNHTKDI | 179 | TNDVRRL | | |
| 180 | FNDAKQW | 181 | NNHTKGV | 182 | TNEDKGL | | |

TABLE 2
N2KR5 motif containing AAV9 7-mer variants enriched through in vivo transduction
screens in C57BL/6J and BALB/cJ mice using CMV-AAV-Express.

| SEQ ID | AA | SEQ ID | AA | SEQ ID | AA | SEQ ID | AA |
|---|---|---|---|---|---|---|---|
| NO | sequence | NO | sequence | NO | sequence | NO | sequence |
| 183 | ANAVRAI | 184 | GNSIRDT | 185 | LNSSRIP | 186 | SNAIKST |
| 187 | ANDIRPW | 188 | GNSIRTI | 189 | LNSVRHV | 190 | SNATRQV |
| 191 | ANHIRSL | 192 | GNSLRAY | 193 | LNTIRST | 194 | SNATRSI |
| 195 | ANTGRHN | 196 | GNSMRHM | 197 | LNTIRTN | 198 | SNEIRLV |
| 199 | ANYARND | 200 | GNSSRNL | 201 | LNTTRPI | 202 | SNFIRSA |
| 203 | DNAVRPW | 204 | GNSTRDH | 205 | LNTTRQV | 206 | SNHIRLA |
| 207 | DNEIRRW | 208 | GNSVKPS | 209 | LNTVRDI | 210 | SNHIRTL |
| 211 | DNHIRSQ | 212 | GNSVKQF | 213 | LNTVREP | 214 | SNHVRAI |
| 215 | DNHVKSL | 216 | GNSVRDF | 217 | LNTVRHI | 218 | SNHVRFM |
| 219 | DNKVKPT | 220 | GNSVRGW | 221 | LNVIRDP | 222 | SNHVRTS |
| 223 | DNNTRSV | 22 | GNSVRPV | 225 | LNYTRSS | 226 | SNNGRYM |
| 227 | DNRTKAL | 228 | GNTIRDI | 229 | MNDVRSV | 230 | SNNVRHY |
| 231 | DNRVRSP | 232 | GNTTKST | 233 | MNHIRTT | 234 | SNQTRSV |
| 235 | DNSTRAT | 236 | GNTVKDP | 237 | MNNARPY | 238 | SNSTKIL |
| 239 | DNSTRWS | 240 | GNTVKLL | 241 | MNQARNP | 242 | SNSTRSV |
| 243 | DNSVRLT | 244 | GNTVRDT | 245 | MNTIRSY | 246 | SNSVRAM |
| 247 | DNTVRGS | 248 | GNVIKTW | 249 | MNTVRDY | 250 | SNSVRDR |
| 251 | DNTWKAA | 252 | GNVIRSH | 253 | NNALKPY | 254 | SNTIKNL |
| 255 | ENATRSL | 256 | GNVTRST | 257 | NNASKGW | 258 | SNTIRTI |
| 259 | ENHLRNT | 260 | GNYGRAQ | 261 | NNATRNG | 262 | SNTTKAG |
| 263 | ENHVRNM | 264 | HNMSRIA | 265 | NNFTRGT | 266 | TNAVRGT |
| 267 | ENNTKIT | 268 | INDARTV | 269 | NNNGRSL | 270 | TNELRSY |
| 271 | ENPIRGR | 272 | INESRNR | 273 | NNSARPL | 274 | TNEVRLS |
| 275 | ENRIRDN | 276 | INHLKSA | 277 | NNSTRAG | 278 | TNFTKMT |
| 279 | ENSIKPL | 280 | INNERSK | 281 | NNSTRGG | 282 | TNGTKPT |
| 283 | ENSVRSA | 284 | INNVKSA | 285 | NNSTRGL | 286 | TNGTRNL |
| 287 | ENVVRSK | 288 | INNVRSV | 289 | NNTIRYS | 290 | TNHIRFT |
| 291 | GNATKAT | 292 | INPRKDN | 293 | NNTTRGM | 294 | TNHIRHL |
| 295 | GNAVKST | 296 | INQIRAA | 297 | NNTVRGM | 298 | TNHIRNL |
| 299 | GNAVRTG | 300 | INQIRQV | 301 | NNVIRGF | 302 | TNHSRPV |
| 303 | GNAVRTV | 304 | INSIRDL | 305 | PNDIRLR | 306 | TNMIRNA |
| 307 | GNDIRVR | 308 | INSIRMT | 309 | PNGIKGV | 310 | TNMPRNS |
| 311 | GNDIRYY | 312 | INTVRDR | 313 | PNNLRTP | 314 | TNPSRFA |
| 315 | GNDTRST | 316 | INYQKPA | 317 | PNNVRQY | 318 | TNSIRDQ |
| 319 | GNESRPT | 320 | INYVKHH | 321 | PNSIRRD | 322 | TNSTKAG |
| 323 | GNESRYM | 324 | KNLLRSE | 325 | PNSMRQV | 326 | TNSTRFT |
| 327 | GNEVRYY | 328 | LNDLRSR | 329 | PNSTRNV | 330 | TNSTRGV |
| 331 | GNFTRDL | 332 | LNEIRAV | 333 | PNTIRNV | 334 | TNSVRPV |
| 335 | GNGTRVY | 336 | LNEPRRV | 337 | PNTTRYL | 338 | TNTVRGG |
| 339 | GNHVRDA | 340 | LNEVKLY | 341 | QNDVRYP | 342 | TNVVKQT |
| 343 | GNLGRSS | 344 | LNHMRNT | 345 | QNEIKTY | 346 | TNYTKTL |
| 347 | GNLTKGY | 348 | LNNIRNV | 349 | QNKHREM | 350 | VNATRGG |
| 351 | GNMIRND | 352 | LNNIRQV | 353 | QNNIKAW | 354 | VNATRST |
| 355 | GNNTKAV | 356 | LNNVRPT | 357 | QNRMRND | 358 | VNEVRFQ |
| 359 | GNNTRSA | 360 | LNNVRSP | 361 | QNRQRDT | 362 | VNHDRAR |
| 363 | GNNVKGL | 364 | LNNVRSV | 365 | RNDGRVA | 366 | VNHIRLQ |
| 367 | GNNVKQF | 368 | LNQYRAA | 369 | RNDTRHI | 370 | VNHLREV |
| 371 | GNQTKPF | 372 | LNRVRGD | 373 | RNERRDV | 374 | VNHVRLT |
| 375 | GNRVKET | 376 | LNSARSI | 377 | RNEVRFA | 378 | VNNERSK |
| 379 | GNSARNI | 380 | LNSGRSA | 381 | RNTVKDP | 382 | VNTIRNV |
| 383 | GNSIRAL | 384 | LNSPRNV | 385 | SNAIKAT | 386 | VNTIRSV |
| | | | | | | 387 | YNQMRNT |

TABLE 3
N2KR5 motif containing AAV9 7-mer variants enriched through in vivo transduction
screens in marmosets using hSyn-AAV-Express.

| SEQ ID | AA | SEQ ID | AA | SEQ ID | AA | SEQ ID | AA |
|---|---|---|---|---|---|---|---|
| NO | sequence | NO | sequence | NO | sequence | NO | sequence |
| 388 | ANAERQP | 389 | GNETKSV | 390 | NNMVRTP | 391 | VNTVREF |
| 392 | ANDMRSG | 393 | GNETRML | 394 | NNSIKVG | 395 | VNTVREF |
| 396 | ANDSRAT | 397 | GNGTRLN | 398 | NNSLKEK | 399 | WNANRIN |
| 400 | ANGTKII | 401 | GNHVKQD | 402 | NNTVKNW | 403 | WNENKSM |
| 404 | ANITKSI | 405 | GNISKNI | 406 | NNVSRDS | 407 | YNAAKGV |
| 408 | ANLSKTT | 409 | GNLVKEN | 410 | NNVSRNN | 411 | YNDDRVF |
| 412 | DNAHRTQ | 413 | GNNIRNY | 414 | PNASRDW | 415 | YNERKEV |
| 416 | DNDIRGK | 417 | GNSARAV | 418 | PNNNKNH | 419 | YNESRNL |
| 420 | DNESKKS | 421 | GNVVKHM | 422 | PNTSKFT | 423 | YNEVKAN |
| 424 | DNKTRLT | 425 | GNYVRDH | 426 | QNALRST | 427 | YNHQKHD |
| 428 | DNNGRLT | 429 | HNLDRSN | 430 | QNALRST | 431 | YNLSRDD |
| 432 | DNNVRNV | 433 | HNLDRSN | 434 | QNGDKLP | 435 | YNMGKDH |
| 436 | DNPSRGI | 437 | HNLERQT | 438 | QNNIKDK | 439 | YNSIRNN |
| 440 | DNPVRGI | 441 | HNLERQT | 442 | QNVARNS | 443 | YNSNKPN |
| 444 | DNRHKEG | 445 | HNLHREN | 446 | RNADKGI | 447 | YNTQKYG |
| 448 | DNRIRGD | 449 | INETRNL | 450 | RNDARNS | | |
| 451 | DNSARET | 452 | INGDRAR | 453 | RNGSKSD | | |
| 454 | ENASKAF | 455 | INGSRNL | 456 | RNNVKAD | | |
| 457 | ENAVRSG | 458 | INKEKTI | 459 | RNTEKEQ | | |
| 460 | ENHLRNT | 461 | INMSKSA | 462 | SNDGRSN | | |
| 463 | ENHTRDK | 464 | INMSKSA | 465 | SNDQKDN | | |
| 466 | ENHVKQN | 467 | INSDRGD | 468 | SNEARSL | | |
| 469 | ENITKNV | 470 | KNTSRED | 471 | SNEPRVL | | |
| 472 | ENLERMV | 473 | LNATRNL | 474 | SNGDRSR | | |
| 475 | ENLIKQH | 476 | LNDIRNT | 477 | SNGGRLQ | | |
| 478 | ENLIRSN | 479 | LNDTRYY | 480 | SNGHKLS | | |
| 481 | ENLIRSN | 482 | LNGARIV | 483 | SNMNRIT | | |
| 484 | ENLLRSS | 485 | LNGARTD | 486 | SNMPRDT | | |
| 487 | ENLMRSS | 488 | LNGLRAH | 489 | SNNIRPI | | |
| 490 | ENLVKDD | 491 | LNGMKGA | 492 | SNNMRPA | | |
| 493 | ENNPRAT | 494 | LNGNRYS | 495 | SNNTKNF | | |
| 496 | ENNTKNY | 497 | LNGSRGA | 498 | SNPDRMK | | |
| 499 | ENQHRTF | 500 | LNGTRSL | 501 | SNTIKNL | | |
| 502 | ENTIRNT | 503 | LNLIKNN | 504 | TNAVKFT | | |
| 505 | ENTIRNT | 506 | LNMERTT | 507 | TNAVKTQ | | |
| 508 | ENTMKAH | 509 | LNNIKNT | 510 | TNDHKQY | | |
| 511 | ENYIRDK | 512 | LNSERSY | 513 | TNGGKYL | | |
| 514 | ENYIRDK | 515 | LNSLRGE | 516 | TNGIRNM | | |
| 517 | ENYVRER | 518 | LNVDRLI | 519 | TNGIRPW | | |
| 520 | FNDEKHT | 521 | MNGGKSL | 522 | TNGMRNQ | | |
| 523 | FNDGRTN | 524 | MNGTKAL | 525 | TNGVRNS | | |
| 526 | FNDNKAF | 527 | MNHSKSA | 528 | TNKERFN | | |
| 529 | FNGNKMH | 530 | MNMDRNT | 531 | TNMVKDW | | |
| 532 | FNGTRNT | 533 | MNSIRNT | 534 | TNQTKSI | | |
| 535 | FNNLKID | 536 | MNSLRSD | 537 | TNVVKAG | | |
| 538 | FNQMKNV | 539 | MNSTKIW | 540 | TNYTKDP | | |
| 541 | FNSDKSR | 542 | NNAPKSS | 543 | TNYTKDP | | |
| 544 | FNTHREG | 545 | NNGMRSS | 546 | VNAIRSQ | | |
| 547 | FNVTRVQ | 548 | NNHMRHD | 549 | VNEVRNY | | |
| 550 | GNATKDQ | 551 | NNLTRSL | 552 | VNSNKHD | | |

TABLE 4
N2KR5 motif containing AAV9 7-mer variants enriched through in vitro transduction
screening on hBMVECs using CMV-AAV-Express.

| SEQ ID | AA | SEQ ID | AA | SEQ ID | AA | SEQ ID | AA |
|---|---|---|---|---|---|---|---|
| NO | sequence | NO | sequence | NO | sequence | NO | sequence |
| 553 | ANHIRSL | 554 | INHIRML | 555 | LNYTRSS | 556 | SNLRRTE |
| 557 | ANLARVT | 558 | INHLKSA | 559 | MNEVRYA | 560 | SNMPRNN |
| 56 | ANTGRHN | 562 | INHMRGA | 563 | MNHIRTT | 564 | SNNGRYM |
| 565 | ANYKRET | 566 | INMKRVP | 567 | MNNARPY | 568 | SNNVRHY |
| 569 | DNARRTL | 570 | INPRKDN | 571 | MNQIRAA | 572 | SNPTRQY |
| 573 | DNAVRPW | 574 | INQIRAA | 575 | MNQSRGW | 576 | SNRQREL |
| 577 | DNAVRSK | 578 | INQIRQV | 579 | MNRSRAE | 580 | SNSHKGW |
| 581 | DNEIRRW | 582 | INSIRMT | 583 | MNRTRFE | 584 | SNTGKSW |
| 585 | DNERRPR | 586 | INSLRLV | 587 | MNSPRSY | 588 | SNTIKNL |
| 589 | DNHIRSQ | 590 | INSPRLF | 591 | MNTIRSY | 592 | SNTVRTI |
| 593 | DNNPRKS | 594 | INSPRVT | 595 | MNTVRAW | 596 | TNDGRSR |
| 597 | DNPNKRQ | 598 | INSQRTP | 599 | MNTVRDY | 600 | TNFGRTT |
| 601 | DNVRRSN | 602 | INSTRYP | 603 | MNVLKKG | 604 | TNHIRFT |
| 605 | ENHLRNT | 606 | INSYKGA | 607 | NNALKPY | 608 | TNHIRNL |
| 609 | ENRSKNN | 610 | INTSRSA | 611 | NNFSRNG | 612 | TNMIRNA |
| 613 | ENSIRMY | 614 | INTVRDR | 615 | NNLNKYL | 616 | TNMPRNS |
| 617 | ENVVRSK | 618 | INTVRSS | 619 | NNMPRGG | 620 | TNPARYG |
| 621 | FNGGRMG | 622 | KNAIKLG | 623 | NNNVRFL | 624 | TNPNRSS |
| 625 | FNKTRGP | 626 | KNLLRSE | 627 | NNPLKRV | 628 | TNPSRFA |
| 629 | FNRERNN | 630 | LNERKYT | 631 | NNSARPL | 632 | TNQLRGY |
| 633 | FNTKRDF | 634 | LNERRYQ | 635 | NNSTRAG | 636 | TNQLRRD |
| 637 | FNTLKLG | 638 | LNGGRSW | 639 | NNSTRGG | 640 | TNSGKWS |
| 641 | FNTPRTA | 642 | LNHIRLS | 643 | NNTIRYS | 644 | TNSTRFT |
| 645 | GNARRET | 646 | LNHIRSL | 647 | NNVIRGF | 648 | VNDLRTR |
| 649 | GNAVKST | 650 | LNHMRNT | 651 | NNYPRNM | 652 | VNGHRSN |
| 653 | GNAWKNA | 654 | LNLNRYS | 655 | PNMPRYE | 656 | VNHVRLT |
| 657 | GNDIRVR | 658 | LNMSKYA | 659 | PNNLRNV | 660 | VNMLRDP |
| 661 | GNESRYM | 662 | LNNIRNV | 663 | PNNVRQY | 664 | VNMPRSV |
| 665 | GNEVRYY | 666 | LNNIRQV | 667 | PNRYKDV | 668 | VNNMRYP |
| 669 | GNGVKWI | 670 | LNNLRSP | 671 | PNSIRRD | 672 | VNSPRTP |
| 673 | GNKFKQA | 674 | LNNTKTA | 675 | PNSYKNI | 676 | VNTIRNV |
| 677 | GNLGRSS | 678 | LNNVRMI | 679 | PNTIRNV | 680 | WNAPRNA |
| 681 | GNMIRND | 682 | LNNVRSP | 683 | PNTLRYA | 684 | WNENKRL |
| 685 | GNMPRSN | 686 | LNNVRSV | 687 | QNNIKAW | 688 | WNSPRTN |
| 689 | GNNTKAV | 690 | LNPRKIG | 691 | QNNNRPA | 692 | YNNFKGN |
| 693 | GNNVKQF | 694 | LNQYRAA | 695 | QNRMRND | 696 | YNRNKFE |
| 697 | GNSARNI | 698 | LNRERSA | 699 | QNRQRDT | 700 | YNSGRNT |
| 701 | GNSIRAL | 702 | LNRVRDH | 703 | QNSLRYS | 704 | YNTGRLV |
| 705 | GNSIRDT | 706 | LNRVRGD | 707 | RNDTRHI | | |
| 708 | GNSIRTI | 709 | LNSARSI | 710 | RNFERAN | | |
| 711 | GNSSRNL | 712 | LNSGRSA | 713 | RNGFREL | | |
| 714 | GNSVRGW | 715 | LNSLRHP | 716 | RNPAKTG | | |
| 717 | GNTIRDI | 718 | LNSVRHV | 719 | RNQDRTT | | |
| 720 | GNVIRSH | 721 | LNTFKAA | 722 | RNVDRST | | |
| 723 | GNVPRVF | 724 | LNTIRST | 725 | RNYIKSD | | |
| 726 | GNVTRST | 727 | LNTLRTI | 728 | SNDGRYY | | |
| 729 | GNYGRAQ | 730 | LNTSRSW | 731 | SNHIRLA | | |
| 732 | GNYSRMD | 733 | LNTVRHI | 734 | SNHIRTL | | |
| 735 | HNDTKRY | 736 | LNYNKGY | 737 | SNHVRAI | | |
| 738 | INAPRTA | 739 | LNYTRPA | 740 | SNHVRFM | | |

TABLE 5
N2KR5 motif containing AAV9 7-mer variants enriched through in vitro transduction
screening on mBMVECs using CMV-AAV-Express.

| SEQ ID NO | AA sequence | SEQ ID NO | AA sequence | SEQ ID NO | AA sequence |
|---|---|---|---|---|---|
| 741 | ANEVRRG | 742 | LNHIRSL | 743 | QNRMRND |
| 744 | ANHIRSL | 745 | LNHMRNT | 746 | QNTARFM |
| 747 | ANNNRNY | 748 | LNNIRNV | 749 | RNDTRHI |
| 750 | ANYKRET | 751 | LNNIRQV | 752 | RNYIKSD |
| 753 | CNGTKRE | 754 | LNNVRMI | 755 | SNHIRTL |
| 756 | CNRSRDG | 757 | LNNVRSV | 758 | SNHVRAI |
| 759 | DNARRTL | 760 | LNPRKIG | 761 | SNHVRTS |
| 762 | DNAVRSK | 763 | LNPVRNA | 764 | SNLRRTE |
| 765 | DNEIRRW | 766 | LNQYRAA | 767 | SNNVRHY |
| 768 | DNHIRSQ | 769 | LNRVRDH | 770 | SNPTRQY |
| 771 | ENHLRNT | 772 | LNRVRGD | 773 | SNRQREL |
| 774 | ENPPRPR | 775 | LNSARSI | 776 | SNSIRFI |
| 777 | ENRSKNN | 778 | LNSGRSA | 779 | SNTIKNL |
| 780 | ENSIRMY | 781 | LNSRREV | 782 | SNTIRTI |
| 783 | ENYGRGA | 784 | LNSVRHV | 785 | SNTVRTI |
| 786 | GNAVKST | 787 | LNTFKAA | 788 | TNFGRTT |
| 789 | GNDIRVR | 790 | LNTIRST | 791 | TNHIRFT |
| 792 | GNDIRYY | 793 | LNTIRTN | 794 | TNHIRNL |
| 795 | GNEVRYY | 796 | LNTIRVV | 797 | TNMIRNA |
| 798 | GNGVKWI | 799 | LNTVRDI | 800 | TNSTRFT |
| 801 | GNMIRND | 802 | LNTVRHI | 803 | TNVLRGF |
| 804 | GNNTKAV | 805 | LNYTRSS | 806 | VNHIRLQ |
| 807 | GNNVKQF | 808 | MNDVRSV | 809 | VNHVRLT |
| 810 | GNSARNI | 811 | MNHIRTT | 812 | VNNERSK |
| 813 | GNSIRAL | 814 | MNHPRMI | 815 | VNSPRSI |
| 816 | GNSIRDT | 817 | MNNARPY | 818 | VNTIRNV |
| 819 | GNSIRTI | 820 | MNQSRGW | 821 | VNTIRSV |
| 822 | GNSMRHM | 823 | MNRTRFE | 824 | WNSPRTN |
| 825 | GNSSRNL | 826 | MNSPRSL | 827 | YNRNKFE |
| 828 | GNSVRGW | 829 | MNTIRSY | 830 | YNSGRNT |
| 831 | GNTIRDI | 832 | MNTVRAW | 833 | YNTGRLV |
| 834 | GNVIRSH | 835 | MNTVRDY | | |
| 836 | GNVMRTT | 837 | NNASKGW | | |
| 838 | GNVTRST | 839 | NNFSRNG | | |
| 840 | GNYGRAQ | 841 | NNNVRFL | | |
| 842 | HNMSRIA | 843 | NNPVRIP | | |
| 844 | INHLKSA | 845 | NNSARPL | | |
| 846 | INHMRGA | 847 | NNSTRAG | | |
| 848 | INMPRDT | 849 | NNSTRGG | | |
| 850 | INNARIP | 851 | NNTIRYS | | |
| 852 | INQIRQV | 853 | NNVIRGF | | |
| 854 | INSPRLF | 855 | PNHVRIA | | |
| 856 | INSPRVS | 857 | PNNLRNV | | |
| 858 | INSPRVT | 859 | PNNVRQY | | |
| 860 | INTSRSA | 861 | PNSIRFS | | |
| 862 | INTVRDR | 863 | PNSIRRD | | |
| 864 | INTVRSS | 865 | PNTIRNV | | |
| 866 | KNAIKLG | 867 | PNTLRYA | | |
| 868 | KNNLREY | 869 | QNLGRYV | | |
| 870 | LNEVKLY | 871 | QNNIKAW | | |

TABLE 6
N2KR5 motif containing AAV9 7-mer variants enriched through in vitro transduction
screening on human CMEC/D3 cells using CMV-AAV-Express.

| SEQ ID | AA | SEQ ID | AA | SEQ | AA | SEQ ID | AA |
|---|---|---|---|---|---|---|---|
| NO | sequence | NO | sequence | ID NO | sequence | NO | sequence |
| 872 | ANGTRGH | 873 | INHVRCV | 874 | LNYNKGY | 875 | SNHIRLA |
| 876 | ANHIRSL | 877 | INIIRVP | 878 | LNYTRPA | 879 | SNHIRTL |
| 880 | ANKNKPV | 881 | INITRNH | 882 | LNYTRSS | 883 | SNHRRME |
| 884 | ANRDRYT | 885 | INMKRVP | 886 | MNHIRTT | 887 | SNHVRAI |
| 888 | ANSQRHS | 889 | INPKRTA | 890 | MNHPRMI | 891 | SNHVRFM |
| 892 | ANYKRET | 893 | INQIRAA | 894 | MNHYRPQ | 895 | SNHVRTS |
| 896 | CNKPKDN | 897 | INSIRMT | 898 | MNLSKYP | 899 | SNKNRWE |
| 900 | DNARRVT | 901 | INSKKNA | 902 | MNMPRTS | 903 | SNLRRTE |
| 904 | DNAVRSK | 905 | INSPRVT | 906 | MNNARPY | 907 | SNNGRYM |
| 908 | DNEIRRW | 909 | INSTRYP | 910 | MNNSRMP | 911 | SNNVRHY |
| 912 | DNERRPR | 913 | INSVRIP | 914 | MNQSRGW | 915 | SNRDRNS |
| 916 | DNHIRSQ | 917 | INSYKGA | 918 | MNRSRAE | 919 | SNRNRDY |
| 920 | DNNPRKS | 921 | INTVRSS | 922 | MNRTRFE | 923 | SNSHKGW |
| 924 | DNVRRSN | 925 | INYQKPA | 926 | MNSPRSL | 927 | SNSIRFI |
| 928 | ENDKKNK | 929 | INYVKHH | 930 | MNSPRSY | 931 | SNTGKSW |
| 932 | ENMPRPT | 933 | KNAIKLG | 934 | MNTIRSY | 935 | SNTIKNL |
| 936 | ENNGRRN | 937 | KNALKSS | 938 | MNTVRAW | 939 | SNTIRTI |
| 940 | ENNRRQM | 941 | KNDERHR | 942 | MNVLKKG | 943 | TNERKYL |
| 944 | ENRQKYT | 945 | KNGGKPN | 946 | NNFSRNG | 947 | TNFGRTT |
| 948 | ENRSKNN | 949 | KNHNKPG | 950 | NNILRVG | 951 | TNHIRFT |
| 952 | ENSLRHR | 953 | KNLMRID | 954 | NNMPRGG | 955 | TNHIRHL |
| 956 | FNGGRMG | 957 | KNPIKSS | 958 | NNPLKRV | 959 | TNHIRHP |
| 960 | FNKTRGP | 961 | LNDSRRP | 962 | NNPVRIP | 963 | TNHIRNL |
| 964 | FNRERGP | 965 | LNERRYQ | 966 | NNSTRAG | 967 | TNKTRPA |
| 968 | FNRERNN | 969 | LNHARYP | 970 | NNSTRGG | 971 | TNMPRNS |
| 972 | FNTKRDF | 973 | LNHGRTA | 974 | NNTIRYS | 975 | TNPARYG |
| 976 | FNTPRTA | 977 | LNHIRLS | 978 | NNTYRSS | 979 | TNQLRRD |
| 980 | GNAVKST | 981 | LNHIRSL | 982 | NNVIRGF | 983 | TNSGKWS |
| 984 | GNAWKNA | 985 | LNHIRTS | 986 | PNEYKAR | 987 | TNSIRLE |
| 988 | GNDIRVR | 989 | LNHMRNT | 990 | PNHPRHL | 991 | TNSLRHP |
| 992 | GNKFKQA | 993 | LNIRRGE | 994 | PNMPRYE | 995 | TNSLRSI |
| 996 | GNLGRSS | 997 | LNKLRGP | 998 | PNNLRTP | 999 | TNSTRFT |
| 1000 | GNLQRYQ | 1001 | LNLNRYS | 1002 | PNNVRQY | 1003 | VNEVRMA |
| 1004 | GNMIRND | 1005 | LNMPRTN | 1006 | PNRYKDV | 1007 | VNHIRLQ |
| 1008 | GNNCKAT | 1009 | LNMSKYA | 1010 | PNSLRAI | 1011 | VNHVRLT |
| 1012 | GNNTKAV | 1013 | LNNIRNV | 1014 | PNSLRER | 1015 | VNLHRSG |
| 1016 | GNNVKQF | 1017 | LNNIRQV | 1018 | PNSMRQV | 1019 | VNNLRTL |
| 1020 | GNSARNI | 1021 | LNNVRSP | 1022 | PNTIRNV | 1023 | VNNMRYP |
| 1024 | GNSIRAL | 1025 | LNNVRSV | 1026 | PNTLRFA | 1027 | VNPNRSG |
| 1028 | GNSIRDT | 1029 | LNPRKIG | 1030 | QNKHREM | 1031 | VNSLRQY |
| 1032 | GNSIRTI | 1033 | LNPVRNA | 1034 | QNNIKAW | 1035 | VNTGKGW |
| 1036 | GNSLRAY | 1037 | LNQYRAA | 1038 | QNNLKYL | 1039 | VNTIRNV |
| 1040 | GNSSRNL | 1041 | LNRERSA | 1042 | QNRMRND | 1043 | VNTPRHS |
| 1044 | GNSVRGW | 1045 | LNRVRDH | 1046 | QNRQRDT | 1047 | WNAPRNA |
| 1048 | GNTIRDI | 1049 | LNRVRGD | 1050 | QNSLRYS | 1051 | WNEYRSS |
| 1052 | GNTYRDY | 1053 | LNSARSI | 1054 | QNTARFM | 1055 | WNHPRAA |
| 1056 | GNVIRSH | 1057 | LNSLRHP | 1058 | RNDGRVA | 1059 | WNNFRPS |
| 1060 | GNVPRVF | 1061 | LNSPRDR | 1062 | RNEVRFA | 1063 | WNPGRAG |
| 1064 | GNVTRST | 1065 | LNSPRFV | 1066 | RNEVRFG | 1067 | WNSNRFE |
| 1068 | GNYGRAQ | 1069 | LNSPRIT | 1070 | RNFNKND | 1071 | WNSPRNT |
| 1072 | HNDTKRY | 1073 | LNSRREV | 1074 | RNMAKAP | 1075 | WNSPRTN |
| 1076 | HNEERKR | 1077 | LNSVRHV | 1078 | RNNGRPQ | 1079 | YNAHRGA |
| 1080 | INAPRTA | 1081 | LNTFKAA | 1082 | RNNPKPL | 1083 | YNMPRGA |
| 1084 | INDLRTP | 1085 | LNTIRST | 1086 | RNPAKTG | 1087 | YNNFKGN |
| 1088 | INHIRML | 1089 | LNTPRST | 1090 | RNVDRST | 1091 | YNQMRNT |
| 1092 | INHLKSA | 1093 | LNTSRSW | 1094 | RNYIKSD | 1095 | YNRNKFE |
| 1096 | INHMRGA | 1097 | LNTVRHI | 1098 | SNFIRSA | 1099 | YNTGRLV |

TABLE 7

| | | | | | Enhanced | Enhanced | |
|---|---|---|---|---|---|---|---|
| | | | | | Transduction | Transduction | |
| | | | SEQ | | in BABLc/J | In Human | |
| | Parental | Peptide | ID | Dominant | | Endothelial | |
| Capsid | Vector | Insertion | NO: | CNS | Strain? | Cell Lines? | Reference |
| BI30 | AAV9 | NNSTRGG | 1 | Endothelial Cells | Yes | Yes- | |
| | | | | | | (hBMVECs & | |
| | | | | | | hCMEC/D3) | |
| PHP.V1 | AAV9 | TALKPFL | 1100 | Endothelial Cells, | No | Yes-(HBMEC) | [1] |
| | | | | Astrocytes | | | |
| PHP.V2 | AAV9 | TTLKPFL | 1101 | Endothelial Cells, | Unknown | Unknown | [1] |
| | | | | Astrocytes | | | |
| PHP.eB | AAV- | [DG]TLA | 1102 | Neurons, | No | No-(hBMVECs | [2] |
| | PHP.B | VPFK | | Astrocytes, | | & hCMEC/D3) | |
| | | | | Oligodendrocytes, | | | |
| | | | | Endothelial Cells | | | |
| PHP.B | AAV9 | TLAVPFK | 1103 | Neurons, | No | Unknown | [3] |
| | | | | Astrocytes, | | | |
| | | | | Oligodendrocytes, | | | |
| | | | | Endothelial Cells | | | |
| BR1 | AAV2 | NRGTEWD | 1104 | Endothelial Cells | Unknown | No- | [4] |
| | | | | | | (hCMEC/D3) | |
| PPS | AAV2 | DSPAHPS | 1105 | Endothelial Cells | Unknown | No- | [5] |
| | | | | | | (hCMEC/D3) | |

### REFERENCES

[1] S.R. Kumar et al., “Multiplexed Cre-Dependent Selection Yields Systemic AAVs for Targeting Distinct Brain Cell Types,” Nat Methods, vol. 17, pp. 541-550, 2020.
[2] K. Y. Chan et al., “Engineered AAVs for Efficient Noninvasive Gene Delivery to the Central and Peripheral Nervous Systems,” Nat Neurosci, vol. 20, no. 8, pp. 1172-1179, 2017.
[3] B. E. Deverman et al., “Cre-Dependent Selection Yields AAV Variants for Widespread Gene Transfer to the Adult Brain,” Nat Biotechnol, vol. 34, no. 2, pp. 204-209, 2016.
[4] J. Körbelin et al., “A Brain Microvasculature Endothelial Cell-Specific Viral Vector With the Potential to Treat Neurovascular and Neurological Diseases,” EMBO Mol Med, vol. 8, no. 6, pp. 609-25, 2016.
[5] Y. H. Chen, M. Chang, and B. L. Davidson, “Molecular Signatures of Disease Brain Endothelia Provide New Sites for CNS-Directed Enzyme Therapy,” Nat Med, vol. 15, no 10, pp. 1215-1219, 2009.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of treating a subject at risk for, or suffering from, a lysosomal storage disease (LSD) comprising;
administering to a subject in need thereof, a therapeutically effective amount of a recombinant adeno-associated virus (rAAV) capsid comprising a vector, the vector comprising a transgene encoding a polypeptide effective to treat the LSD,
wherein the rAAV comprises a modified capsid comprising a n-mer motif that increases transduction of endothelial cells of a CNS vasculature, the n-mer motif comprising or consisting of X1-N-X3-X4-X5-X6-X7, wherein X5 is independently selected from K or R, and X1, X3, X4, X6 and X7 are independently selected from any amino acid, optionally wherein an overall charge of the n-mer motif at neutral pH is between 0 and +2.

2. The method of claim 1, wherein the LSD is selected from the group consisting of Mucopolysaccharidosis (MPS), Sphingolipidosis, Oligosaccharidosis, Neuronal ceroid lipofuscinosis, Sialic acid disorders, Mucolipidosis, Lysosomal Acid lipase deficiency infantile and childhood/adult types, Pompe disease, Danon disease, Wolman's disease. and Cystinosis.

3. The method of claim 2, wherein the LSD is a MPS.

4. The method of claim 3, wherein the MPS is selected from the group consisting of Hurler syndrome (MPS I), Hunter syndrome (MPS II), San Filippo syndrome A (MPS IIIA), San Filippo B (MPS IIIB), San Filippo C (MPS IIIC), San Filippo D (MPS IIID), Morquio syndrome A (MPS IVA), Morquio syndrome B (MPS IVB), Scheie syndrome (MPS V), Maroteaux-Lamy syndrome (MPS VI), Sly syndrome (MPS VII), hyaluronidase deficiency (MPS IX), and Hurler-Scheie syndrome.

5. The method of claim 2, wherein the MPS is: Hurler Syndrome and the transgene encodes IDUA; Hunter Syndrome and the transgene encodes iduronate 2-sulfatase (IDS); San Filippo Syndrome A and the transgene encodes SGSH; San Filippo Syndrome B and the transgene encodes NAGLU; San Filippo Syndrome C and the transgene encodes HGSNAT; San Filippo Syndrome D and the transgene encodes GNS; Morquio Syndrome A and the transgene encodes GALNS; Morquio Syndrome B and the transgene encodes GLB1; Scheie Syndrome and the transgene encodes IDUA; Maroteaux-Lamy Syndrome and the transgene encodes Aryl sulfatase B; Sly Syndrome and the transgene encodes GUSB; Hurler-Scheie Syndrome and the transgene encodes IDUA; or any combination thereof.

6. The method of claim 1, wherein the vector comprises one or more repeat elements that reduce or eliminate expression of the transgene in a non-endothelial cell of the CNS vasculature.

7. The method of claim 6, wherein the one or more repeat elements are a hepatocyte-selective miR-122 repeat element.

8. The method of claim 1, wherein X1, X3, X4, X6, and X7 is independently selected from the following groups:
X1 is selected from the group consisting of G, M, T, S, N, D, L, H, P, I, V, Q, Y, W, F, A, E;
X3 is selected from N, S, T, H, D, A, Y, M, Q, E, R, G, V;
X4 is selected from T, V, I, A, M, S, H, W, N;
X6 is selected from N, S, G, D, P, T, H, Q, A, Y; and
X7 is selected from T, Y, W, N, V, I, H, M, S, G, A, Q, F, D, P, R, L.

9. The method of claim 1, wherein X1, X3, X4, X6, and X7 are independently selected from the following groups:
X1 is selected from the group consisting of G, M, T, S, N, D;
X3 is selected from the group consisting of N, S, T, H, D;
X4 is selected from the group consisting of T, V, I, A;
X6 is selected from the group consisting of N, S, G, D, P; and
X7 is selected from T, Y, W, N, V, I, H, M, S, G, A, Q, F, D, P, R, L.

10. The method of claim 1, wherein X1 is R or K and X3, X4, X6 and X7 are D or E.

11. The method of claim 1, wherein:
X1 is not R, K, or C;
X3 is not W, F, K, C, I, P or, L;
X4 is not Y, G, P, D, C, Q, R, K, E, F, L, or R;
X6 is not R, I, W, V, F, C, L, E, or K; or
X7 is not C, K, E.

12. The method of claim 1, wherein the n-mer motif is selected from one of Table 1 to Table 6.

13. The method of claim 1, wherein the n-mer motif is NNSTRGG (SEQ ID NO: 1), GNSARNI (SEQ ID NO: 2), GNSVRDF (SEQ ID NO: 3), or a combination thereof.

14. The method of claim 1, wherein the n-mer motif is part of a viral capsid protein.

15. The method of claim 14, wherein the n-mer motif is located between two amino acids of the viral capsid protein such that the n-mer is external to a viral capsid.

16. The method of claim 15, wherein the viral capsid protein is an AAV viral capsid protein.

17. The method of claim 16, wherein the n-mer motif is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

18. The method of claim 17, wherein the AAV capsid polypeptide comprises one or more mutations.

19. The method of claim 18, wherein the one or more mutations comprise K449R of AAV9, or in an analogous position in an AAV1, AAV2, AAV3, AAV4, AAV5, AAV6, AAV7, AAV8, AAV rh.74, AAV rh.10 capsid polypeptide.

20. A rAAV comprising a vector, the vector comprising a transgene encoding IDS, wherein the rAAV comprises a modified capsid comprising a n-mer motif that increases transduction of endothelial cells of a CNS vasculature, the n-mer motif comprising or consisting of X1-N-X3-X4-X5-X6-X7, wherein X5 is independently selected from K or R, and X1, X3, X4, X6 and X7 are independently selected from any amino acid, optionally wherein an overall charge of the n-mer motif at neutral pH is between 0 and +2.

21. The rAAV of claim 20, wherein the vector comprises one or more repeat elements that reduce or eliminate expression of the transgene in a non-endothelial cell of the CNS vasculature, wherein the one or more repeat elements are a hepatocyte-selective miR-122 repeat element.

22. The rAAV of claim 20, wherein X1, X3, X4, X6, and X7 is independently selected from the following groups:
X1 is selected from the group consisting of G, M, T, S, N, D, L, H, P, I, V, Q, Y, W, F, A, E;
X3 is selected from N, S, T, H, D, A, Y, M, Q, E, R, G, V;
X4 is selected from T, V, I, A, M, S, H, W, N;
X6 is selected from N, S, G, D, P, T, H, Q, A, Y; and
X7 is selected from T, Y, W, N, V, I, H, M, S, G, A, Q, F, D, P, R, L.

23. The rAAV of claim 20, wherein X1, X3, X4, X6, and X7 are independently selected from the following groups:
X1 is selected from the group consisting of G, M, T, S, N, D;
X3 is selected from the group consisting of N, S, T, H, D;
X4 is selected from the group consisting of T, V, I, A;
X6 is selected from the group consisting of N, S, G, D, P; and
X7 is selected from T, Y, W, N, V, I, H, M, S, G, A, Q, F, D, P, R, L.

24. The rAAV of claim 20, wherein X1 is R or K and X3, X4, X6 and X7 are D or E.

25. The rAAV of claim 20, wherein:
X1 is not R, K, or C;
X3 is not W, F, K, C, I, P or, L;
X4 is not Y, G, P, D, C, Q, R, K, E, F, L, or R;
X6 is not R, I, W, V, F, C, L, E, or K; or
X7 is not C, K, E.

26. The rAAV of claim 20, wherein the n-mer motif is selected from one of Table 1 to Table 6.

27. The rAAV of claim 20, wherein the n-mer motif is NNSTRGG (SEQ ID NO: 1), GNSARNI (SEQ ID NO: 2), GNSVRDF (SEQ ID NO: 3), or a combination thereof.

28. The rAAV of claim 20, wherein the n-mer motif is part of a viral capsid protein.

29. The rAAV of claim 28, wherein the n-mer motif is located between two amino acids of the viral capsid protein such that the n-mer is external to a viral capsid.

30. The rAAV of claim 29, wherein the viral capsid protein is an AAV viral capsid protein.

31. The rAAV of claim 30, wherein the n-mer motif is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

32. The rAAV of claim 31, wherein the AAV capsid polypeptide comprises one or more mutations.

33. The rAAV of claim 32, wherein the one or more mutations comprise K449R of AAV9, or in an analogous position in an AAV1, AAV2, AAV3, AAV4, AAV5, AAV6, AAV7, AAV8, AAV rh.74, AAV rh.10 capsid polypeptide.

34. A pharmaceutical composition comprising the rAAV of claim 20.
